(19) United States
(12) Patent Application Publication
(10) Pub. No.: US 2025/0134829 A1
(43) Pub. Date: May 1, 2025

(54) LOW-DOSE, STABLE-RELEASE TRANSDERMAL PREPARATION AND PATCH, AND METHOD FOR THEIR PRODUCTION

(71) Applicants: PÉCSI TUDOMÁNYEGYETEM, Pécs (HU); BUDAPESTI MUSZAKI ÉS GAZDASÁGTUDOMÁNYI EGYETEM, Budapest (HU)

(72) Inventors: Erika PINTÉR, Pécs (HU); Lajos BOTZ, Pécs (HU); Gábor POZSGAI, Bicsérd (HU); Szabolcs LÁSZLÓ, Budapest (HU); Ödön WAGNER, Budaörs (HU)

(21) Appl. No.: 19/004,082

(22) Filed: Dec. 27, 2024

Related U.S. Application Data
(63) Continuation-in-part of application No. PCT/HU2023/050042, filed on Jun. 27, 2023.
(60) Provisional application No. 63/615,177, filed on Dec. 27, 2023.

(30) Foreign Application Priority Data
Jun. 27, 2022 (HU) P2200237

(51) Int. Cl. A61K 9/70; A61K 9/16; A61K 31/165; A61K 31/196; A61K 31/34; A61P 25/02; A61P 29/00

(52) U.S. Cl. CPC A61K 9/7069; A61K 9/1611; A61K 9/1623; A61K 9/7092; A61K 31/165; A61K 31/196; A61K 31/34; A61P 25/02; A61P 29/00

(57) ABSTRACT

The invention relates to a topical, preferably transdermal preparation and patch, which can deliver its active ingredient content into the skin with near zero-order kinetics, as well as a method for their production. The active ingredient is preferably an NSAID or a capsaicinoid. In particular, the invention relates to a transdermal patch comprising a low dose of capsaicin, which, when placed on the skin, ensures the release of the active ingredient over a long period of time, preferably for 4 to 24 hours, more preferably for 6-12 hours, with near zero-order kinetics. The invention further relates to the production of these preparations. The invention further relates to the use of these preparations to relieve pain, preferably acute pain, and/or chronic inflammatory and neuropathic pain, and as a warm-up patch during sports activities.

This is a continuation-in-part of international application PCT/HU2023/050042, filed Jun. 27, 2023, and also claims benefit of U.S. provisional application 63/615,177, filed Dec. 27, 2023, the entire disclosures of which are hereby incorporated by reference herein.

### FIELD OF THE INVENTION

The invention relates to a transdermal preparation, which can deliver its active ingredient content into the skin with near zero-order kinetics, as well as a method for its production.

The invention relates to a transdermal patch containing a low dose of capsaicin, which when placed on the skin provides a long-term release of the active ingredient, preferably for 4-24 hours, more preferably for 6-12 hours, with near zero-order kinetics. The invention also relates to the production of these preparations. The invention also relates to the use of these preparations to relieve pain, preferably acute pain and/or chronic inflammatory and neuropathic pain, and as a warm-up patch during sports activities.

### TECHNICAL BACKGROUND

It is a long-known fact that transdermal preparations are able to deliver their active ingredient content—with appropriate release kinetics—into the skin, or into the human body through the skin. Medicine exploits this characteristic, which delivers suitable active ingredients into the human body using this convenient and effective method.

Transdermal therapeutic systems (TTS) or transdermal drug delivery systems (TDDS) provide an excellent mode of convenient, accurate, safe and painless dosing in drug therapy [Hoffman, A. S. 2008, Tanner, T. and Marks R., 2008]. Dermal absorption systems can be categorized on the basis of their structure or chemical composition. According to the structure, they can be adhesive polymer dispersion-based type, membrane-controlled type, polymer matrix diffusion-controlled and “micro-reservoir” type systems [Goswami et al. (2015); Hadgraft J. and Lane E. M. 2006]. Based on their material, there are hydrophilic organic copolymers (e.g. polyols, polyethers, etc.) and silicone-based systems (hydrophobic or modified amphiphilic structure).

From the point of view of drug release, membrane-controlled systems possess the most favorable characteristics. Their only disadvantage is that the drug is in a liquid phase under the control membrane. The TTS cannot be cut, the dose rate cannot be changed. Adhesive polymer dispersion systems are excellent in this respect, but the kinetics of drug release are not optimal. The two characteristics are well combined in “micro-reservoir” type systems, but these are expensive to produce. This type of TTS is thicker than the others and release is controlled by diffusion through the polymer matrix [Pastore, M. N. et al., 2015]. The construction of the polymer matrix from organic polymers is complicated due to solubility problems and other chemical properties.

Several patents describe dosage forms that are suitable for controlled, transdermal administration of the active ingredient. For example, U.S. Pat. Nos. 4,466,953, 3,946,106, 6,316,023B1, 5,948,433, 9,226,902B2, South African patent ZA94414B, European patent EP2310001B1, etc. The solutions known in the art use different raw materials and different structures (adhesive-polymer carrier, multilayer, microencapsulated, matrix, microporous or non-porous membrane controlled, etc.) to achieve the controlled release of the active ingredient from the preparation.

On the other hand, it is known that many raw materials suitable for transdermal purposes are used (acrylate, silicone, various copolymers), but silicones stand out among them, which—due to their biocompatibility—are very well tolerated by the human body.

Hungarian patents with registration numbers HU197519 and HU210931 use silicone raw material. The Hungarian patent HU197519 describes a transdermal patch that consists of 2-6 silicone layers and each layer contains an increasing concentration of the active ingredient from the layer in contact with the skin. The thickness of the layers is 0.1-3 mm, their active ingredient content is 0-45% by weight, preferably 0-25% by weight of active ingredient; however, the patch does not contain the active ingredient in a saturation concentration. The patches also contain liquid and/or solid excipients. The surface opposite to the application side is covered with a moisture-proof layer and coated with a material that secures it to the skin surface. With this structure, the active ingredient release with near zero-order kinetics is achieved. In the solution, at least two layers contain active ingredient. The described transdermal patch is 0-40% by weight.

The solution described in Hungarian patent with registration number HU210931 is built from a matrix containing two silicone layers. The layer in contact with the skin does not contain an active ingredient and is in contact with the underlying layer containing the active ingredient with a wavy interface. This interface, which has an increased size compared to the layer in contact with the skin, ensures the uniform release of the active ingredient from the preparation with near zero-order kinetics.

Goswami et al. (2015) present the types of transdermal drug delivery systems in their article. They mention that in matrix-type systems, the polymer can contain the active ingredient in dissolved or dispersed form. They also note that the transdermal preparation may contain other excipients—e.g. enhancer—but this is not a mandatory element. They also draw attention to the fact that if the TDDS layer contains the active ingredient in a saturated or near-saturated concentration, there is a risk of crystallization, which Goswami et al. consider harmful—against this, for example, crystallization inhibitor excipients can be used in the patches.

Hadgraft et al. (2006) describe that the use of a reservoir-type transdermal therapeutic system can ensure that there is always active ingredient capable of being released. An essential component of one type of the reservoir-type systems is the polymer membrane that controls release, which allows the active ingredient to pass through in a specified amount. In another type of the reservoir systems, the adhesive (tape) layer itself forms the matrix, as well, and the active ingredient is contained within it.

Ichikawa and Sugiura (2013) describe a transdermal preparation of the active ingredient tulobuterol. The described patch consists of only one layer, an adhesive layer, which contains tulobuterol in both molecular and crystallized form. The release of the active ingredient is extended, but does not occur with zero-order kinetics.

In summary, it can be said about the characteristic features of the earlier, known systems that the membrane-controlled patches, which are suitable for implementing near zero-order kinetics on a different principle, however, for example, they are not suitable for dosing by cutting; in the drug-in-adhesive layer type patches, the release of the active ingredient is not regulated; the microreservoir-type patches are not suitable either for achieving dosing by cutting and their flexibility is also weak, while the polymer matrix diffusion-controlled patches according to the state of the art are not suitable for achieving near zero-order kinetics.

Chronic pain is a problem that affects about 20% of the population worldwide, which seriously impairs the quality of life for the individual patients, and on the other hand, represents a significant burden for both the health care system and society. It is not only a major detriment to the quality of life of the patients affected, but also the leading cause of years with disability [GBD 2019 Diseases and Injuries Collaborators, 2020]. The most common causes of chronic pain are locomotor diseases (lower back pain, rheumatoid arthritis, chronic degenerative polyarthritis), neuropathic conditions of various origins (caused by diabetes, herpes virus infection, traumatic nerve injury, neurodegenerative diseases), or other pathologies, e.g. complex regional pain syndrome. Chronic pain conditions with a component of neural origin (neuropathic component), such as neuropathic pain caused by diabetes, herpes virus infection, traumatic nerve injury, neurodegenerative diseases, etc., are particularly problematic.

The analgesics most often used to relieve chronic pain are unfortunately not effective in all cases, and significant side effects must be expected in the case of their long-term use. Therefore, intensive research is currently underway in the pharmaceutical industry to develop new types of analgesic active ingredients and to improve the mode of application of painkillers.

In the development of chronic pain, the activation of the so-called capsaicin-sensitive sensory nerve endings and neurogenic inflammation mediated by proinflammatory substances released from the nerve endings also play an important role, making it an excellent target for new types of pain therapy.

The TRPV1 (Transient Receptor Potential Vanilloid 1) receptor is one of the first described signal-receiving structures of capsaicin-sensitive sensory nerve endings, which after activation releases Na⁺ and Ca²⁺ ions into the cells, which generates a sensation of pain and can mediate multiple pain stimuli. TRPV1 has a complex sensor function, in addition to irritants from the outside world, it is activated or sensitized by high heat (>43° C.), mechanical and chemical stimuli that cause pain. Among its activators, the most well-known is the active component of chili pepper, capsaicin, which is why it was initially referred to as the “capsaicin receptor”. A high dose (6-8%) of capsaicin locally causes a chronic pain-relieving effect after the initial very strong pain, it temporarily disables the capsaicin-sensitive sensory nerve endings, which effect lasts for about 2-3 months. This effect of capsaicin is called “desensitization”. An example for this mechanism is the transdermal patch under the brand name Qutenza (Canadian patent numbered CA02314326), which contains a very large amount of capsaicin (8%), and is used to relieve so-called neuropathic pain caused by nerve injuries. After exposure to large amounts of capsaicin, the pain receptors in the skin become insensitive to various stimuli, while the function of other nerves that do not express TRPV1 does not change. In a comparative study, the high-concentration (high-dose) patch of Qutenza was significantly more effective than a control patch with a low dose of capsaicin (0.04%). However, a significant disadvantage of the Qutenza patch is that it can only exert its effect locally and, due to the loss of function of the sensory nerves, it desensitizes the given skin surface for a long time. Somatostatin, which is released from sensory nerve endings that are continuously activated with capsaicin concentrations much lower (<1%) than the dose that inactivates the TRPV1 receptor, exerts effective pain-relieving and anti-inflammatory effects.

Capsaicin-containing transdermal patches are commercially available. Topical capsaicinoid therapy effectively alleviates pain in several diseases, including rheumatoid arthritis, osteoarthritis, low back pain, and neuropathic pain [Deal, C. L. et al., 1991, Cameron M. et al., 2009; De Leon-Casasola, O. 2011, Chrubasik, S. et al. 2010, also Guedes, V. et al. 2008][1-5]; and it increases blood flow of soft tissues before sports activity to achieve a warming up effect. Capsaicin-containing creams and ointments are also available, but they have the disadvantage of contaminating the hands and potentially irritating the mucous membranes and eyes. This is especially problematic for individuals wearing contact lenses. Topical non-steroidal anti-inflammatory drugs (NSAIDs) are most commonly applied to relieve pain caused by osteoarthritis. The topical use of low-dose capsaicin and NSAIDs is an accepted therapy in the treatment of osteoarthritic pain, but the exact mechanism is still under investigation [Persson M. S. N. et al., 2018]. Ercan et al. found that in a carrageenan-induced inflammation model, topical application of patches containing capsaicinoids and several other antioxidant agents enhanced the anti-inflammatory effect of diclofenac in rats, while capsaicin alone did not. Therefore, the authors assume that the enhanced effect of the topical patch on the anti-inflammatory action of NSAIDs is due to other active substances of red pepper other than capsaicin [Ercan et al. 2013].

International application WO2020263643A1 suggests the combined topical use of capsaicin and diclofenac, but does not describe a patch containing them together. Chinese patent CN107296852 claims the combined use of several active ingredients, including capsaicin and diclofenac, in a polyacrylate patch, however, it is not clear what effect the patch has and which active ingredient it is due to. U.S. Pat. No. 5,665,378A describes a polyacrylate copolymer-based patch and cream containing the active ingredients ibuprofen (an NSAID), capsaicin, and pamabrom, but only the pain-relieving effect of the cream is reported in a single patient. It seems that the state of the art does not describe the actual use of low-dose capsaicin and diclofenac together in pain-relieving patches, especially dissolved in a liquid excipient in silicone matrix, or in patches in which no other active ingredient is present, e.g. antioxidant or diuretic, possibly penetration-enhancing excipient.

The beneficial effect of capsaicin relies on activation of transient receptor potential vanilloid 1 (TRPV1) ion channels on peptidergic nociceptor nerve endings and subsequent release of neuropeptides [Bley, K. R. et al. 2010; Kaale, E et al., 2002]. Our previous study established that topical capsaicinoid (nonivamide) therapy diminishes the chronic low back pain. Nonivamide proved to be efficient in functional tests such as ODI (effect of pain on daily life) and VAS (visual analog scale of the pain sensation) [Horváth, Boros et al. Neuropeptides, 2014]. According to clinical trials executed on patients suffering from low back pain, nonivamide treatment exerts analgesic action inducing threefold increase of the plasma level of the antinociceptive and anti-inflammatory neuropeptide somatostatin, which might play a role in the pain-relieving effect. Although these commercially available patches and creams may contain capsaicin in small doses (less than 1% by weight); but these only administer the active ingredient into the surface layer of the skin, and the method of application cannot be controlled either. Furthermore, in many cases they also contain some other active ingredient besides capsaicin (e.g. menthol). Topically applied creams and ointments containing small amounts of capsaicin have shown low efficiency in several studies, presumably due to poor absorption from the skin or due to poor patient cooperation. Another disadvantage of these products is that they contaminate the hands or they irritate mucous membranes and the eyes, which is especially problematic for individuals who wear contact lenses.

It has been assumed that low-dose capsaicin can reduce pain by itself, since, in addition to pain-causing substances (inflammatory neuropeptides), anti-inflammatory, pain-relieving substances (somatostatin, endogenous opioids) are also released and, reaching the systemic circulation, exert their effects throughout the body. A number of capsaicin-containing drugs and medicinal preparations—among them plasters—that can be applied primarily locally are also known, which are mainly used to relieve joint and muscle pains (e.g., Dr Chen—Capsiplast, Seyitler Kimya—Fastplast, Hansaplast—ABC warmepflaster, etc.). These administer capsaicin only into the surface layer of the skin, the method of administration is not regulated.

At the same time, in several studies, low-concentration, topically applied capsaicin did not prove to be clearly effective compared to a placebo preparation [Peppin J. F. and Pappagallo M 2014]. Low-dose creams showed a weak effect, presumably due, in addition to the low dose, to poor skin absorption and compliance factors [Knotkova H et al. 2008].

A Cochrane review of published randomized clinical trials of low-dose topical capsaicin concluded that “although topically applied capsaicin has moderate to poor efficacy in the treatment of chronic musculoskeletal or neuropathic pain, it may be useful as an adjunct or sole therapy for a small number of patients who are unresponsive to, or intolerant of, other treatments” [Mason et al. 2004].

In the above-mentioned Hungarian patent HU197519, a transdermal patch is described, which consists of 2 to 6 layers of silicone rubber, which optionally contain the active ingredient. Capsaicin is not mentioned among the active ingredients.

European patent numbered EP1865933B1 describes a patch that is for use in the treatment of neuropathic pain and the active ingredient of which is capsaicin or its analogue that the patch contains in a small amount, less than 1% by weight (for example, about 0.04% by weight), however, the capsaicin is not released with near zero-order kinetics. The patch also contains a penetration enhancer (diethylene glycol monoethyl ether), which ensures that the active ingredient reaches the deeper layers of the skin within a relatively short time. The patch only needs to be applied once, for a maximum of 120 minutes, which causes pain relief for at least 1 to 3 months. Thus, conceptually, in the patch the method of administering the active ingredient and treating the patient does not differ from high-dose patches, it only includes a reduction of the dose for those on whom such dose already has a sufficient effect. The patch can also optionally contain a self-adhesive matrix, which comprises, for example, an amine-resistant polysiloxane. It may also contain excipients.

Moon et al. (2017) in their article investigated the efficacy and safety of low-dose (0.625% and 1.25%) capsaicin patches compared to conventional 0.075% creams and placebo for the treatment of peripheral neuropathic pain. To their surprise, according to their results, the application of the 0.625% patch is likely to be effective and safe, and these patches may be suitable for use in place of the high concentration (8%) patches, but further studies are needed. On the other hand, they did not detail the construction (structure) of the patches they worked with, the person skilled in the art did not receive any guidelines for this from the article; only the dose was given: 0.625% (50 μg/cm²) or 1.25% (100 μg/cm²) [Moon, J.-Y. et al., 2017].

None of the above solutions allow the low-dose capsaicin to be released at an even rate, over a prolonged period (e.g. over 4 to 24 hours), and the pain-relieving effect of the patch to only last as long as the patch is on the skin.

The aim of the invention is to develop a transdermal patch that releases the active ingredient over a long period of time (in particular 4 to 24 hours), with near zero-order kinetics; and at the same time, not to be necessary to contain the active ingredient in increasing concentrations or to have a wavy interface between the individual layers. Furthermore, the aim of the invention is to produce such transdermal patches.

The aim of the invention is to develop a transdermal patch that ensures the release of the active ingredient low-dose capsaicin over a long period of time—without the risk of damaging the compound—with near zero-order kinetics and ensures its penetration through the skin. On the other hand, the aim of the invention is to produce a transdermal patch containing a low dose of capsaicin.

Furthermore, the aim of the invention is to prolong the pain-relieving effect of a transdermal patch containing diclofenac. The aim of the invention is to produce a transdermal patch containing diclofenac that alleviates pain for up to 6 hours.

### BRIEF DESCRIPTION OF THE INVENTION

The invention relates to a topical, preferably transdermal preparation for administering an active ingredient topically, preferably through the skin, said preparation comprising
a matrix, preferably a silicone matrix comprising an active ingredient in the form of a pure substance, preferably in a solid form, which is preferably formed in the form of a layer (preferably a matrix layer comprising a solid active ingredient),
an active ingredient dispersed in the matrix in the form of a pure substance, preferably in the form of solid particles (granules),
the saturated solution of the active ingredient in the matrix,
an excipient, preferably a liquid excipient, which controls the solubility of the active ingredient in dissolved form in the matrix, preferably adjusts the saturation concentration to a predetermined level,
if desired, a surfactant, preferably for dispersing the excipient or the active ingredient in a polar matrix, preferably in a silicone matrix,
in addition, optionally, a solid excipient dispersed in the form of granules to ensure accurate dosing and homogenous dispersion of the active ingredient,
wherein the active ingredient leaving the matrix during administration through the skin is replaced by the dissolution of the active ingredient dispersed in the form of a pure substance, preferably in the form of solid particles, in such a way that the active ingredient in solution preferably remains in saturated solution for at least 4 hours, whereby the release of the active ingredient from the matrix (and thus its administration through the skin) occurs evenly (stably), preferably with near zero-order kinetics, preferably for at least 4, more preferably for at least 6 hours.

Optionally, the liquid excipient is a solvent in which the active ingredient is dissolved.

Preferably, the invention relates to a transdermal preparation, preferably a transdermal preparation formed as part of a patch, for administering an active ingredient through the skin, said preparation comprising
a silicone matrix comprising an active ingredient in solid form, which is formed in the form of a layer (preferably a matrix layer comprising a solid active ingredient),
an active ingredient dispersed in the silicone matrix in the form of solid particles (granules),
the saturated solution of the active ingredient in the silicone matrix,
liquid excipient, which controls the solubility of the active ingredient in dissolved form in the matrix, and in which the active ingredient is dissolved,
if desired, a surfactant for dispersing the excipient or the active ingredient in a polar matrix, preferably in a silicone matrix,
in addition, optionally, a solid excipient dispersed in the form of granules to ensure accurate dosing and homogenous dispersion of the active ingredient,
wherein the active ingredient leaving the matrix during administration through the skin is replaced by the dissolution of the active ingredient dispersed in the form of a pure substance, preferably in the form of solid particles. Preferably, the active ingredient is released from the matrix (and thus administered through the skin) with near zero-order kinetics, preferably for at least 4, more preferably for at least 6 hours. Preferably, the active ingredient in solution remains in saturated solution for at least 4 hours, more preferably for at least 6 hours.

In a preferred embodiment, the excipient controlling the solubility of the active ingredient in the preparation is selected from the group consisting of monohydric and polyhydric alcohols, preferably polyhydric alcohols.

In a preferred embodiment, the solubility of the excipients and, optionally, the solubility (or dispersion) of polyhydric alcohols, their dispersion in the matrix are aided with a surfactant. The surfactant is preferably a surfactant defined below in the brief description of the invention, preferably a nonionic surfactant, in a preferred embodiment a polysorbate. Particularly preferably, the polysorbate is polysorbate 20. The surfactant is preferably a surfactant defined in the detailed description of the invention.

The solubility of the excipient in the matrix, when the matrix has already been polymerized, optionally refers to its presence or mixing or distribution.

In another preferred embodiment, the preparation comprises a solid excipient dispersed in the form of granules, preferably a solid excipient selected from the group of amorphous colloidal silica, preferably hydrophilic colloidal silica; and/or an inactive solid excipient, preferably calcium carbonate.

In another preferred embodiment, the preparation comprises (on the topical administration side, preferably on the skin side of the matrix comprising the active ingredient present in the form of a pure substance, preferably a solid active ingredient) a single additional matrix, preferably an additional matrix layer, which does not comprise active ingredient in the form of a pure substance, preferably does not comprise solid active ingredient (additional or regulator matrix). The additional matrix, preferably matrix layer comprises
an excipient, preferably a liquid excipient,
if desired, a surfactant, preferably for dispersing the excipient or the active ingredient in a polar matrix, preferably in a silicone matrix,
optionally, the solution of the active ingredient in the matrix.

In a particularly preferred embodiment, the preparation comprises capsaicin or a capsaicin analogue as an active ingredient, preferably comprises capsaicin. Preferably, the liquid excipient is at least a polyhydric alcohol.

Preferably, the solid excipient is powdered calcium carbonate or (preferably amorphous) colloidal silica.

Preferably, the transdermal preparation comprises capsaicin as an active ingredient, polyhydric alcohol as a liquid excipient, and a solid excipient.

In a particularly preferred embodiment, the preparation comprises capsaicin or a capsaicin analogue, preferably capsaicin, as active ingredient; and comprises an NSAID, preferably diclofenac. Preferably, the concentration ratios are specified for capsaicin in the description.

Preferably, the matrix (preferably silicone matrix) does not comprise a penetration enhancer.

Preferably, the preparation (and thus the matrix) is formed in a flat shape, in a layer thickness of at most 0.1 mm to 1 mm, preferably 0.1 mm to 0.8 mm or 0.2 mm to 1 mm, in particular 0.2 mm to 0.6 mm.

In an embodiment, the preparation is formed in the form of a layer, and arranged in contact with one of the planes of the matrix layer comprising the active ingredient, it also comprises a matrix layer that does not comprise the active ingredient in solid form, preferably an additional silicone layer, the composition of which is preferably the same as the matrix layer comprising the active ingredient, but without the active ingredient.

In a preferred embodiment, the transdermal preparation formed as part of a patch according to the invention comprises a single matrix layer comprising a solid active ingredient.

In another preferred embodiment, the transdermal preparation formed as part of a patch according to the invention comprises a matrix layer comprising a solid active ingredient, and a single additional matrix layer on the topical administration side, preferably on the skin side, of the matrix layer comprising the solid active ingredient, which does not comprise active ingredient in the form of a pure substance, preferably does not comprise solid active ingredient (additional or regulator matrix).

Preferably, the preparation is formed as part of a transdermal patch.

In a preferred embodiment, the patch comprises a carrier layer (1), a layer (2) comprising a solid active ingredient as a matrix layer, and an adhesive layer (4) for fixing it on the skin (5). In another preferred embodiment, the patch comprises a carrier layer (1), a layer (2) comprising a solid active ingredient as a matrix layer, and a regulator layer (3), which does not comprise solid active ingredient, and an adhesive layer (4).

Preferably, the preparation can achieve a uniform release of the active ingredient, preferably a prolonged (preferably for at least 4 hours, more preferably for at least 6 hours) release of the active ingredient with near zero-order kinetics, without having to form a wavy interface between the layers or without having to use the active ingredient in increasing concentrations in each layer or without having to use a membrane regulating the passage of the active ingredient on the boundary plane (interface) of the preparation.

Preferably, during the production of the preparation, if the preparation is formed in the form of multiple layers in contact with each other along their interface (preferably their boundary plane), the active ingredient is only introduced into one layer. The preparation and thus, optionally, the patch does not comprise an active ingredient concentration that changes, e.g. increases, through the layers.

In a preferred embodiment, the preparation comprises capsaicin or a capsaicin analogue as active ingredient.

Preferably, the preparation comprises the capsaicin or capsaicin analogue at a saturation concentration of 0.05% to 1% (i.e., this is the concentration of the saturated solution and does not include the amount of the pure solid form). More preferably, the preparation comprises capsaicin or a capsaicin analogue, preferably capsaicin, in a saturation concentration of at most 0.5%, more preferably less than 0.4%, preferably at most 0.3%. More preferably, the preparation comprises capsaicin or a capsaicin analogue in a saturation concentration of 0.05% to 0.5%, in particular 0.1% to 0.4%, 0.05% to 0.3%. In a particularly preferred variant, the preparation comprises capsaicin in a saturation concentration of more than 0.1% and less than 0.35%, preferably 0.15% to 0.3%, in particular 0.2% to 0.3%. Preferably, the matrix is a silicone matrix, in particular a silicone layer. In a preferred embodiment, the silicone oligomer used to form the silicone matrix is a silicone elastomer.

Preferably, the liquid and/or solid excipients promoting the diffusion of capsaicin or capsaicin analogue in the matrix are present in the silicone matrix at 4% to 20%.

In a preferred embodiment, the liquid excipient is selected from the group consisting of monohydric and polyhydric alcohols, preferably polyhydric alcohols, in particular trihydric or polyhydric alcohols, and surfactants.

In a preferred embodiment, the solid excipient is an active solid excipient, preferably hydrophilic colloidal silica, preferably amorphous colloidal silica; and/or an inactive solid excipient, preferably calcium carbonate.

In a preferred embodiment, the raw material of the silicone matrix (preferably silicone layers) is silicone cross-linking by condensation and/or addition method. The silicone defined in the description, preferably in the brief description of the invention, can be used. Particularly preferably, the crosslinker is a crosslinker described or defined in the description, preferably below.

In a preferred embodiment, the thickness of the individual silicone layers is 0.1 mm to 0.5 mm each.

The invention also relates to the preparation according to the invention comprising capsaicin or a capsaicin analogue for topical use in pain relief; preferably for topical use through the skin. Preferably the pain is neuropathic pain, preferably peripheral pain. Particularly preferably, the pain is postherpetic pain, e.g. neuropathic pain. In a preferred variant, the pain is selected from
acute pain,
chronic inflammatory pain,
neuropathic pain.

Particularly preferably, the preparation is for use for pain relief limited to the time of application.

The invention also relates to the preparation according to the invention comprising a capsaicinoid, preferably capsaicin, for use in pain relief with prolonged release of the active ingredient.

The invention also relates to the preparation according to the invention comprising a capsaicinoid, preferably capsaicin or a capsaicin analogue, for use in pain relief with a stable, near zero-order kinetic release of the active ingredient.

The invention also relates to the preparation according to the invention comprising a capsaicinoid, preferably capsaicin or a capsaicin analogue, for use in pain relief for at least 4 hours, preferably for at least 6 hours, particularly preferably for 4 to 24, 4 to 12, particularly for 6 to 12 hours, with prolonged, preferably near zero-order kinetic release of the active ingredient.

Preferably, the preparation, preferably the matrix within it, does not comprise a penetration enhancer.

Particularly preferably, in the matrix layer of the patch according to the invention, the active ingredient is present in a proportion of at most 0.5% by weight, more preferably at most 0.4% by weight, particularly preferably at most 0.3% by weight, preferably in a saturation concentration. Particularly preferably, this is true for all active ingredients.

Particularly preferably, the matrix layer of the patch comprises two types of active ingredients. Particularly preferably, the two types of active ingredients are capsaicinoid, preferably capsaicin or a capsaicin analogue, and NSAID, preferably diclofenac.

Particularly preferably, in the patch according to the invention, the active ingredient is present (as a measured concentration) in a surface ratio of at most 1 mg/cm², preferably 0.5 mg/cm², particularly preferably 0.4 mg/cm², even more preferably 0.3 mg/cm² with reference to the surface of the patch in contact with the skin.

Particularly preferably, the patch according to the invention comprises one or more active ingredients.

Particularly preferably, the active ingredient is a capsaicinoid, preferably capsaicin or a capsaicin analogue and/or an NSAID, preferably diclofenac.

Particularly preferably, in the patch comprising capsaicin and diclofenac, diclofenac is present in a proportion of at most 0.4% by weight.

Particularly preferably, in the patch comprising capsaicin and diclofenac, the capsaicin is present in a surface ratio of at most 0.5 mg/cm² as a measured concentration, with reference to the surface of the patch in contact with the skin.

Particularly preferably, in the patch comprising capsaicin and diclofenac, the diclofenac is present in a surface ratio of at most 0.5 mg/cm² as a measured concentration, with reference to the surface of the patch in contact with the skin.

Preferably, the preparation for use in pain relief comprises the capsaicinoid, preferably capsaicin, in a saturation concentration of at most 0.5%, more preferably less than 0.4%, preferably at most 0.3%. More preferably, the preparation comprises the capsaicinoid, preferably capsaicin or a capsaicin analogue, in a saturation concentration of 0.05% to 0.5%, particularly 0.1% to 0.4%, 0.05% to 0.3%. In a particularly preferred variant, the preparation comprises the capsaicin in a saturation concentration of more than 0.1% and less than 0.35%, preferably 0.15% to 0.3%, in particular 0.2% to 0.3%.

Preferably, the preparation according to the invention comprises capsaicin and diclofenac. Particularly preferably, the active ingredient content of the patch according to the invention essentially consists of capsaicin and diclofenac. Particularly preferably, the patch according to the invention comprises only capsaicin and diclofenac as active ingredients.

Particularly preferably, in the preparation comprising capsaicin and diclofenac, the capsaicin is present in a saturation concentration of at most 0.5%, more preferably less than 0.4%, preferably at most 0.3%.

Particularly preferably, in the patch comprising capsaicin and diclofenac, the diclofenac is present in a saturation concentration of at most 0.5%, more preferably less than 0.4%, preferably at most 0.3%.

In a preferred embodiment, the capsaicin is present in the patch at a concentration of at most 5 mg/g, based on the matrix layer comprising the active ingredient in solid form or on the matrix layer and the regulator layer. Preferably, it is present in a concentration of at most 2.3 mg/g, particularly preferably in a concentration of 1 mg/g to 2.3 mg/g, in a preferred variant, in a concentration of at most 1 mg/g.

The invention relates to methods of treatment using the preparation according to the invention, by applying the preparation topically, preferably through the skin. Preferably, the preparation is used in the form of a patch.

The invention relates to a transdermal drug delivery system (TDDS) or a transdermal therapeutic system (TTS), preferably a transdermal patch for delivering an active ingredient with near zero-order kinetics, which comprises any of the above-defined preparations formed in the form of a layer, and comprises a pressure-sensitive adhesive silicone tape layer in contact with the plane of the preparation formed in the form of a layer (on its surface), optionally it comprises a pressure-sensitive adhesive silicone tape layer in contact with the plane of the layer that does not comprise the active ingredient in solid form (if present) (on its surface).

The pressure-sensitive adhesive silicone tape layer optionally comprises liquid and/or solid excipients.

Preferably, the transdermal patch comprises one or two silicone matrix layer(s) and a pressure-sensitive adhesive layer, wherein the pressure-sensitive adhesive layer is closest to the skin during application; wherein
the transdermal patch is formed in such a way that it only comprises solid active ingredient in a single silicone matrix layer (if two silicone matrix layers are present, in the silicone matrix layer farther away from the topical application site, preferably the skin, in the layer comprising the solid active ingredient) and
the transdermal patch comprises the active ingredient in both dissolved and solid form in the layer comprising the solid active ingredient; and
the transdermal patch releases the active ingredient with zero-order kinetics for 4 to 24 hours, preferably for 6 to 12 hours.

Preferably, the active ingredient is capsaicinoid, preferably capsaicinoid with a concentration of at most 1% by weight, preferably capsaicin or an analogue thereof (which is used in the manner and concentration defined in the description, preferably in the brief description of the invention or in the claims).

Preferably, the transdermal patch comprises
a) a silicone matrix layer comprising an active ingredient in the form of a pure substance, preferably a solid active ingredient, which comprises the following:
   cross-linking silicone as raw material,
   optionally a crosslinker,
   an active ingredient, wherein the active ingredient is present both in dissolved and solid form,
   a liquid excipient (polar solvent) for the dissolution of capsaicin, preferably dihydric or trihydric, preferably trihydric alcohol, e.g. glycerol,
   optionally a surfactant (emulsifier) for the uniform distribution of the polar solvent within the matrix,
   optionally a solid excipient; and
b) a pressure-sensitive adhesive layer; and
c) optionally, a silicone matrix layer comprising no solid active ingredient between the silicone matrix layer comprising a solid active ingredient according to a) and the pressure-sensitive adhesive layer according to b), wherein the silicone matrix layer comprising no solid active ingredient comprises the following:
   cross-linking silicone as raw material,
   a liquid excipient, preferably dihydric or trihydric, preferably trihydric alcohol, e.g. glycerol,
   optionally a crosslinker,
   optionally a surfactant (emulsifier).

Optionally, the liquid excipient is a solvent in which the active ingredient is dissolved.

In a preferred embodiment, the excipient controlling the solubility of the active ingredient in the preparation is selected from the group consisting of monohydric and polyhydric alcohols, preferably polyhydric alcohols.

In a preferred embodiment, the solubility of the excipients and, optionally, the solubility (or dispersion) of polyhydric alcohols, their dispersion in the matrix are aided with a surfactant. The surfactant is preferably a surfactant defined below in the brief description of the invention, preferably a nonionic surfactant, in a preferred embodiment a polysorbate. Particularly preferably, the polysorbate is polysorbate 20. The surfactant is preferably a surfactant defined in the detailed description of the invention.

The solubility of the excipient in the matrix, when the matrix has already been polymerized, optionally refers to its presence or mixing or distribution.

In a preferred embodiment of the invention, the active ingredient is a capsaicinoid, preferably at most 1% by weight of capsaicinoid, preferably at most 1% by weight of capsaicin (or optionally a capsaicin analogue), wherein the active ingredient is present in both dissolved and solid form in the layer comprising the solid active ingredient.

In a preferred embodiment of the invention, the active ingredient is selected from the following:
non-steroidal anti-inflammatory drugs (NSAIDs),
particularly preferably diclofenac,
nitrites and nitrates, nitrate esters, sugar esters or esters of sugar derivatives, preferably nitrate esters of sugars and sugar derivatives, nitrate esters of heterocyclic compounds, preferably nitrate esters of hydroxy-substituted heterocyclic compounds, preferably nitrates of heterocycles with O-containing ring, preferably nitrate esters of hydroxy-substituted O-containing heterocycles; preferably medicines for cardiovascular diseases,
particularly preferably isosorbide dinitrate (ISDN).

In a preferred embodiment of the invention:

The thickness of each layer of the transdermal patch is independently 0.1 mm to 0.6 mm or the layer thickness defined herein.

The solid capsaicin dispersed in the matrix has been mixed with a powder diluted (triturated) inert excipient (e.g. calcium carbonate) prior to being mixed into the matrix.

Particularly preferably, the amount of excipients in total is 4% to 20% by weight.

Particularly preferably, the liquid excipient is a polar solvent.

Particularly preferably, the polar solvent is saturated with the active ingredient in the layer comprising the active ingredient in solid form.

Particularly preferably, the polar solvent is a monohydric or polyhydric alcohol, preferably a trihydric alcohol, preferably glycerol. Preferably, the polar solvent remaining in the patch is a non-volatile solvent, i.e. its concentration does not change during storage and application of the patch. In particular, if alcohol, in particular monovalent alcohol, is used, it is not volatile. If a volatile solvent is used, it is allowed to evaporate during the preparation of the patch so that the dissolution conditions of the finished patch do not change.

Preferably, the transdermal patch does not comprise a penetration enhancer.

Particularly preferably, in this previous and further embodiments, the crosslinking silicone raw material is silicone rubber silicone raw material crosslinking by condensation and/or addition method, preferably crosslinking at room temperature (RTV-2), e.g. polydimethylsiloxane-α,ω-diol, preferably component A of Elastosil RT-601 and component B of Elastosil RT-601, or polydimethylsiloxane-α,ω-diol (with a viscosity of 5000 mPas or 20000 mPas).

Particularly preferably, the crosslinker is, for example, an Oxam crosslinking catalyst or a TES-40 prehydrolyzed tetraethoxysilane crosslinker.

Particularly preferably, the emulsifier is a polysorbate, preferably polysorbate 20.

Particularly preferably, the solid excipient is a (preferably amorphous) colloidal silica, preferably hydrophilic colloidal silica.

Particularly preferably, the solid excipient is a solid microcrystalline carbonate, preferably calcium carbonate.

In another preferred embodiment, the solid excipient is a saccharide, in particular glucose or lactose.

In an embodiment, the invention relates to a TTS, preferably a transdermal patch for the stable delivery of an active ingredient, characterized in that it comprises a preparation according to the invention, wherein the matrix of the preparation comprises a single silicone layer comprising the active ingredient in the form of a pure substance, preferably in a solid form (said silicone layer comprising the active ingredient both in the form of its saturated solution prepared with a liquid excipient, and in the form of a pure substance, preferably a solid substance (preferably in a solid silicone matrix)),
and optionally comprises a single, additional silicone layer layered on top of it, said additional silicone layer comprising no active ingredient in the form of a pure substance, preferably comprising no solid active ingredient,
and the attachment of the preparation consisting of one or two layer(s) to the skin is ensured by a pressure-sensitive adhesive silicone layer (adhesive layer) formed on the surface of the silicone layer comprising the active ingredient in the form of a pure substance, preferably in a solid form, or, if present, on the surface of the additional silicone layer.

Any layer may comprise liquid and/or solid excipients, as well.

The active ingredient is released from the TTS, preferably from the patch, according to the invention with near zero-order kinetics.

Optionally, the additional silicone layer that does not comprise solid active ingredient comprises only a liquid excipient as an excipient; optionally comprises a liquid and/or solid excipient.

Preferably, the thickness of the silicone matrix layers of the transdermal preparation is 0.1 mm to 1 mm, preferably 0.1 mm to 0.8 mm or 0.2 mm to 1 mm, particularly 0.2 mm to 0.6 mm, particularly preferably 0.3 mm to 0.5 mm each.

Preferably, the transdermal patch does not comprise a penetration enhancer.

Preferably, the active ingredient present in the form of a pure substance, preferably solid particles, is in a solid state.

Preferably, it is in solid granular (powder) form, wherein the size of the particles is 5 to 200 microns, preferably 20 to 150 microns, particularly preferably 20 to 125 microns.

The TDDS or TTS, preferably transdermal patch, according to the invention comprises a pressure-sensitive adhesive layer on its surface in contact with the skin.

In another variant, the TDDS or TTS, preferably transdermal patch, comprises the preparation according to the invention in a form layered onto a carrier. Preferably, the carrier is a carrier layer that can be used in the production of patches. In a variant, the carrier layer extends beyond the layers of the preparation, preferably the silicone layers. Preferably, an adhesive, preferably a pressure-sensitive adhesive (PSA), is layered on the skin-contacting surface of the overhanging part of the carrier layer.

Preferably, the liquid excipient is a polar and/or apolar liquid and/or a mixture thereof, and, if desired, an anionic and/or non-ionic surfactant(s) or a mixture thereof.

Particularly preferably, the liquid excipient in the preparation (which is preferably an excipient controlling the solubility of the active ingredient) is a polar liquid, which is preferably selected from the group consisting of monohydric and polyhydric alcohols and surfactants.

In a preferred embodiment, the preparation or patch comprises an apolar excipient. Particularly preferably, the apolar excipient is selected from the group consisting of methyl silicone oils of different viscosities (preferably in the viscosity range of 50 to 350 mPa·s), cyclic (preferably tetracyclic, pentacyclic) polymethylsiloxanes.

In another preferred embodiment, the preparation comprises a solid excipient dispersed in the form of granules, preferably a solid excipient selected from the group consisting of amorphous colloidal silica; and/or inactive solid excipient, preferably calcium carbonate.

In another variant, it comprises a saccharide (carbohydrate) as a solid excipient, preferably lactose or glucose, preferably as a carrier.

Particularly preferably, the silicone layer(s) comprise 1% to 15% of liquid and 0% to 5% of solid excipient.

Particularly preferably, in the preparation according to the invention, the raw material of the silicone layer is a cross-linked polydimethylsiloxane comprising reactive groups or a mixture of polydimethylsiloxanes (preferably see below).

In a preferred embodiment, in order to fix the silicone matrix to the skin, it comprises a pressure-sensitive adhesive layer on the surface of the silicone matrix, which comprises a liquid and optionally solid excipient as defined in the description.

In another preferred embodiment, depending on the properties of the active ingredient, a liquid excipient selected from the group comprising (or consisting of) apolar liquids, preferably silicone oils, cyclic methyl siloxanes, and/or polar liquids, preferably monohydric, dihydric or trihydric alcohols, polyalcohols, polyethers, and/or mixtures thereof, is used as a liquid excipient.

In a preferred embodiment, the pressure-sensitive adhesive layer has a thickness of 0.1 mm to 0.5 mm, has a liquid excipient content of 1% to 15%, preferably 1% to 7%, and has a solid excipient content of 0% to 5%.

Particularly preferably, the amount of excipients in total is 4% to 20% by weight.

Particularly preferably, the liquid excipient is a polar solvent.

Particularly preferably, the polar solvent is saturated with the active ingredient. Particularly preferably, the polar solvent is a monohydric or polyhydric alcohol, preferably a trihydric alcohol, preferably glycerol.

In a particularly preferred variant, the crosslinking silicone is siloxane, preferably polyalkylsiloxane-diol, particularly polydimethylsiloxane-α,ω-diol, preferably polydimethylsiloxane-α,ω-diol (with a viscosity of 5000 mPas or 20000 mPas). This solution is preferred, as a relatively polar environment is formed during cross-linking, thus typically no surfactant is required.

In another variant, the crosslinking silicone raw material is a silicone raw material crosslinking by an addition method, e.g. component A of Elastosil RT-601 and component B of Elastosil RT-601. In this case, a relatively apolar environment is formed during cross-linking, thus typically a surfactant is required.

Particularly preferably, the crosslinker also comprises a catalyst, which comprises a crosslinking agent and a compound that catalyzes crosslinking. Particularly preferably, the catalyst is an oxalaminidate-based catalyst, in particular Oxam crosslinking catalyst.

In a variant, the catalyst is added separately to the crosslinking agent. In a preferred embodiment, the crosslinker is an alkoxysilane crosslinker, for example a tetraethoxysilane crosslinker. Preferably TES-40.

Particularly preferably, the emulsifier is a polysorbate, preferably polysorbate 20.

Particularly preferably, the solid excipient is an amorphous colloidal silica, preferably hydrophilic colloidal silica.

Particularly preferably, the solid excipient is a solid microcrystalline carbonate, preferably calcium carbonate.

In another embodiment, the solid excipient is a saccharide, preferably glucose or lactose.

The invention also relates to a method for producing the transdermal preparation according to the invention, characterized in that the active ingredient is mixed into the silicone raw material in the form of a saturated solution and a solid substance, then it is cross-linked in a thin layer (preferably laid out or spread on a support), and, optionally, onto its surface (preferably after the solidification of the layer comprising the active ingredient) another silicone mixture comprising a liquid and/or solid excipient is layered and cross-linked.

In a variant, the invention also relates to a method for producing the TDDS or TTS according to the invention, preferably a transdermal patch, during which a pressure-sensitive adhesive (PSA) layer comprising a liquid and/or solid excipient is layered on the preparation.

Preferably, the layer comprising the active ingredient is spread on a carrier.

Preferably, a PSA layer is layered on the formed one-layer or, optionally, two-layer silicone matrix.

Preferably, any silicone cross-linking by condensation and/or addition method and/or a mixture thereof are used as silicone raw material. Preferably, the silicone raw materials described in the description, for example in the brief description of the invention or in the examples, can be used.

Preferably, the layers of the silicone matrix are each spread out and cross-linked in a layer thickness of 0.1 mm to 1 mm, preferably 0.1 mm to 0.8 mm or 0.2 mm to 1 mm, particularly 0.2 mm to 0.6 mm, particularly preferably 0.3 mm to 0.5 mm.

Preferably, the liquid excipient is a polar and/or apolar liquid and/or a mixture thereof.

Preferably, 1% to 15% of liquid and 0% to 5% of solid excipient are mixed into the silicone layer(s) before cross-linking.

Preferably, the silicone layer(s) comprise 1% to 7% of liquid and 0% to 5% of solid excipient and the layer thickness (per layer) is 0.1 mm to 0.5 mm.

Preferably, the excipient is any excipient defined in the description.

Preferably, an anionic and/or non-ionic surfactant is used in an amount of 0% to 5%, preferably 1.5% to 4%, for the homogeneous mixing of the liquid excipient in the silicone matrix. Particularly preferably, the surfactant is non-ionic, preferably a polysorbate.

Preferably, the excipient is any excipient defined in the description.

In a preferred embodiment, the pressure-sensitive adhesive (PSA) layer layered on the cross-linked layer—if present, on the layer that does not comprise the active ingredient in the form of a pure substance, preferably in solid form—is a silicone-based adhesive. Preferably, the solvent is allowed to evaporate from the adhesive during the process.

In a preferred embodiment, during the production of the TDDS or TTS, preferably a transdermal patch,
the layer comprising the active ingredient is produced, preferably layered on a carrier,
optionally, an additional layer comprising no active ingredient in solid form is layered on the layer comprising the active ingredient,
optionally, a PSA layer is layered as an additional layer on the layer comprising the active ingredient or, if present, optionally, on the additional layer.

Particularly preferably, the invention relates to a method for producing a releasing transdermal preparation, preferably a patch, characterized in that the active ingredient is mixed into the silicone raw material of the preparation in the form of a saturated solution prepared with a liquid excipient, as well as in the form of a pure substance and
a solid excipient is also added to the mixture, then it is spread on a carrier film—preferably in a layer thickness of 0.2 mm to 0.6 mm—and the layer is cross-linked, then it is covered with a pressure-sensitive adhesive layer comprising liquid and/or solid excipients.

In a preferred embodiment, during the production of the preparation
a powder dilution (trituration) is prepared from the solid form of the active ingredient with the solid granular excipient,
a solution is prepared from the active ingredient, the liquid excipient and, optionally, the surfactant,
crosslinkable silicone components are provided,
a mixture comprising the crosslinkable silicone components, the powder diluted active ingredient and the dissolved active ingredient is prepared,
the silicone components are cross-linked, thereby preparing a silicone matrix comprising the (solid) active ingredient,
wherein the amount of solid and dissolved active ingredient and the amount of liquid excipient are adjusted in such a way that the equilibrium of the solid active ingredient and dissolved active ingredient in the preparation creates a saturated solution of the dissolved active ingredient in the silicone matrix.

In an embodiment, the method according to the invention comprises the following as an additional step,
preparing a solution from the liquid excipient and, optionally, from the surfactant,
providing crosslinkable silicone components,
preparing a mixture comprising the crosslinkable silicone components, the powder diluted active ingredient and the dissolved active ingredient,
layering the mixture on the silicone matrix comprising the (solid) active ingredient,
cross-linking the silicone components, thereby creating a silicone matrix, preferably a regulator silicone matrix or a regulator (silicone) layer.

Preferably, the crosslinking silicone is a silicone defined herein, preferably a siloxane or a siloxane derivative. Preferably, the crosslinking silicone is a siloxane, preferably a polyalkylsiloxanediol, in particular a polydimethylsiloxane-α,ω-diol.

In another embodiment, the crosslinking silicone is Elastosil, preferably component A of Elastosil RT-601 and component B of Elastosil RT-601.

Particularly preferably, the crosslinker also comprises a catalyst, which comprises a crosslinking agent and a compound that catalyzes crosslinking. Particularly preferably, the catalyst is an oxalaminidate-based catalyst, in particular Oxam crosslinking catalyst.

In a variant, the catalyst is added separately to the crosslinking agent. In a preferred embodiment, the crosslinker is an alkoxysilane crosslinker, for example a tetraethoxysilane crosslinker. Preferably TES-40.

Particularly preferably, the emulsifier is a polysorbate (see the description), particularly preferably polysorbate 20.

In a preferred embodiment, the preparation is used in a topical, preferably transdermal patch, wherein the preparation is in a layered form, and a pressure-sensitive adhesive (PSA) layer is layered on the preparation. In a variant that does not comprise a regulator matrix or layer, the PSA layer is layered on the layer comprising the (solid) active ingredient. If desired, the evaporation of the volatile component(s) are waited out or facilitated.

Preferably, the layer comprising the active ingredient is spread on a carrier.

The invention also relates to the use of the active ingredients defined according to the invention for the manufacture of the preparation according to the invention.

The invention also relates to the use of capsaicinoids, preferably capsaicin or capsaicin analogues, defined according to the invention for the manufacture of the preparation according to the invention.

The invention also relates to the use of NSAIDs, preferably diclofenac, for the manufacture of the preparation according to the invention.

The invention also relates to a method for the uniform administration of an active ingredient to a person (preferably a patient) through the skin, for at least 4 hours, preferably for at least 6 hours, during which
a preparation according to the invention comprising the active ingredient is provided,
the preparation is contacted with the surface of the skin of the person for at least 4 hours, preferably for at least 6 hours.

The invention also relates to a method for the uniform administration of an active ingredient to a person (preferably a patient) through the skin, for at least 4 hours, preferably for at least 6 hours, during which
a TDDS or TTS or a patch according to the invention comprising the active ingredient is provided,
the TDDS or TTS or patch comprising the active ingredient is attached to the person's affected skin surface, and
it is left there for at least 4 hours, preferably for at least 6 hours.

The active ingredient of the patch is preferably released for at least 4 hours, more preferably for 6 hours, particularly preferably for 4 to 24 hours, in particular for 6 to 12 hours, with near zero-order kinetics.

The treated person (patient) is preferably a vertebrate, particularly preferably a mammal, particularly preferably a human.

If desired or necessary during the treatment, body hair must be removed and the patch applied in contact with the skin.

### Definitions

In the description, the proportion of the active ingredient and the excipients has been given as a percentage, which means—unless specifically indicated otherwise—a percentage by weight.

“Preparation” in the sense according to the description, refers to a material composition that comprises a matrix and an active ingredient in it together with suitable excipients, and which is suitable for delivering the active ingredient to the site of administration, preferably to the skin, directly or via a regulator layer or a pressure-sensitive adhesive layer.

“Topical preparation” refers to a preparation that is for administering to a given place of the body, i.e. for “topical administration”; preferably topical administration is an administration through a body surface, preferably an administration into or through the skin or into or through a mucosa, preferably an administration through the skin.

“Patch” or “transdermal patch” in the description refers to a preparation or device with a layered arrangement that can be attached, preferably adhered, to a skin surface, preferably a vertebrate, more preferably a mammalian or particularly a human skin surface, and which carries an active ingredient, for contacting the biological substance with the skin or with a biological substance found in the skin. The skin patch preferably comprises the active ingredient in a “layer comprising an active ingredient”, usually in a matrix (“matrix”), which enables controlled release. The patch typically comprises the layer comprising the active ingredient affixed (“attached”) to a carrier layer (“backing layer” or “carrier layer”). Typically, the patch may comprise an additional layer through which the active ingredient must pass in order to come into contact with the skin surface, and thus, optionally, the additional layer may contribute to the regulation of the release of the active ingredient. Optionally, the skin patch further comprises a penetration enhancer (“permeation enhancer”), but preferably it does not.

Preferably, the skin patch is affixed (“attached”) to the skin surface by adhesion (by an “adhesive”), wherein the adhesive device is attached to the carrier layer, e.g. by means of adhesive applied to the area of the carrier layer, which is a layer comprising an active ingredient not attached to a surface or an additional layer layered on top of it.

“Active ingredient” in the description refers to a substance to be used for the benefit of a person (“patient”), preferably a human person, undergoing treatment or examination, which has a biological effect in the person's living body corresponding to the purpose of use.

Active ingredient “capsaicinoid” refers to a compound which is an amide derivative of vanillylamine and which has a similar effect to capsaicin, preferably which are activators of TRPV1 receptors, particularly preferably which cause the release of neuropeptides from the nerve endings during activation, in particular compounds which in low doses (below 1%) cause a sustained, low-intensity activation of the TRPV1 receptor.

“Non-steroidal anti-inflammatory drugs”, abbreviated NSAID (NonSteroidal Anti-Inflammatory Drug) are anti-inflammatory drugs that do not have a sterane skeleton and do not exert their effect on the receptors of steroid hormones (glucocorticoids). NSAIDs are often have pain-relieving effect.

In the description, “pure substance” refers to the presence of the active ingredient in the preparation in a form that does not mix with the solution comprising the active ingredient in dissolved form in the matrix, and is preferably present in another phase. Pure substance preferably refers to the presence of the active ingredient in solid form, preferably in solid granular form, from which the active ingredient can dissolve into the surrounding solution, thereby maintaining a saturated solution of the active ingredient. The surrounding solution is preferably (essentially) a saturated solution during the uniform release of the active ingredient, wherein optionally the liquid excipient is a solvent.

In the description, “matrix” refers to a material whose function is to accommodate another material, preferably an active ingredient. In an embodiment, the matrix is in a solid state and comprises spaces that are not filled with the material of the matrix and is thus suitable for storing the other material, preferably an active ingredient. In an embodiment, the matrix is a polymer in which the other material, preferably an active ingredient, is located in a dissolved state, however, the high viscosity and secondary chemical binding forces present in the solution allow storage. In an embodiment, the matrix is suitable for receiving the active ingredient in the form of a pure substance, preferably in solid granular form, as well.

The matrix of the transdermal patch according to the invention consists of one or, optionally, two layers.

Silicone matrix refers to a polymer matrix that comprises—as described or defined in the description—a silicone in such a way that it is able to receive the active ingredient in a dissolved form with appropriate excipients in a regulated form in terms of its solubility. The silicone matrix according to the invention is suitable for receiving the active ingredient in the form of a pure substance, preferably in solid granular form, as well.

In the description, “silicone layer” refers to a layer that comprises solid silicone in such an amount that it determines its mechanical properties and that is suitable as a matrix for receiving the active ingredient and/or excipients.

According to the description, “uniform” or “stable” release of the active ingredient means that the rate of active ingredient release is essentially constant or only slightly variable, possibly decreasing according to the established measure of the state of the art. According to the description, “uniform” release of the active ingredient is preferably release of the active ingredient with near zero-order kinetics.

The uniform release of the active ingredient, preferably with near zero-order kinetics, means that the desired active ingredient release level, during the time period of the active ingredient release, preferably for at least 4 hours, more preferably for at least 6 hours, remains within a range of ±30%, preferably remains within a range of ±25%, preferably within a range of ±20%, preferably within a range of ±15%, particularly preferably within a range of ±10% compared to the average (average rate or mean value) of the active ingredient release rate. Preferably, the rate of active ingredient release decreases slightly, but there is a period of at least 4 hours, preferably at least 6 hours, during which the decrease in the rate of active ingredient release compared to the value at the beginning of the period remains within a range of at most 30%, preferably within a range of at most 25%, preferably within a range of at most 20%, preferably within a range of at most 15%, particularly preferably within a range of at most 10%.

In a preferred variant, during the near zero-order kinetics, after the topical use of the preparation, preferably the application of the patch, and/or after the start of the administration of the active ingredient, the rate of active ingredient release established after reaching the blood-saturating dose of the active ingredient, remains within a range of ±30%, preferably within a range of ±25%, preferably within a range of ±20%, preferably within a range of ±15%, particularly preferably within a range of ±10% compared to the active ingredient release of the 3^{rd} hour after the start of the administration, for at least 4 hours, more preferably for at least 6 hours.

In a preferred variant, during the near zero-order kinetics, after the topical use of the preparation, preferably the application of the patch, followed by an increasing and then optionally a decreasing transient level of active ingredient release, preferably after a transitional period of at most 5 hours, more preferably of at most 4 hours, particularly preferably of at most 3 hours, the rate of the release of the active ingredient remains within a range of ±30%, preferably within a range of ±25%, preferably within a range of ±20%, preferably within a range of ±15%, particularly preferably within a range of ±10% compared to the rate of active ingredient release measured at the end of the transitional period, for at least 4 hours, more preferably for at least 6 hours.

As used herein, “a” or “an” is to be interpreted as an indefinite article, unless the context indicates otherwise; and if the context permits, the definite article “the” includes plural reference, unless the context indicates otherwise. Thus, if the context so permits, the term “a” or “an” shall mean “one or more”.

The term “comprising” or “including” are to be construed as having a non-exhaustive meaning and allow the addition or involvement of further features or method steps or components in anything or to anything which comprises the listed features or method steps or components. As used herein, the term “consisting essentially of” or “comprising substantially” includes mandatory features or method steps or components listed in a list, e.g. in a claim, and does not exclude that the use, method, composition, or any other subject matter contains additionally other features or method steps or components which do not materially affect the essential characteristics. As used herein, the term “comprising” or “including” can be replaced, if necessary, by the terms “consisting essentially of” or “comprising substantially” without addition of new matter. Selection from a list, for example, the term “selected from” can be replaced by “selected from the group consisting of” if practice so requires and, if the context permits, includes selecting one or more item(s) from the list.

### Abbreviations

HPLC: high-performance liquid chromatography
HSE: heat separated epidermis
IVPT: in vitro permeation test
IVRT: in vitro release test
PBS: phosphate buffer
PDMS: dimethylpolysiloxane
TTS: transdermal therapeutic system

### BRIEF DESCRIPTION OF THE FIGURES

FIG. 1. Modified silicone-polymer-based matrix controlled diffusion TTS, which comprises (A) a carrier layer (1), a layer comprising a solid active ingredient (2), a regulator layer (3), an adhesive layer (4) or (B) a carrier layer (1), a layer comprising a solid active ingredient (2) and an adhesive layer (4).

FIG. 2. Scheme of condensation silicone polymerization.

FIG. 3. Scheme of addition silicone polymerization.

FIG. 4. Apparatus for single-layer spreading of the polymer.

FIG. 5. Flow-through cell.

FIG. 6. Measurement of the thermal pain threshold.

FIG. 7. Measurement of the mechanical pain threshold.

FIG. 8. The cumulative amount of released capsaicin in μg/cm² during 24 hours (* indicates p<0.05, ** indicate p<0.01 and *** indicate p<0.001).

FIG. 9. The cumulative amount of permeated capsaicin in μg/cm² during 24 hours (*** indicate p<0.001).

FIG. 10. The released capsaicin in μg/cm² during 6 hours (in flow-through cell).

FIG. 11. Raman correlation maps for the distribution of capsaicin in human skin after treatment with patches. Untreated skin is also displayed as a control. Color coding of drug concentration: red>yellow>green>blue.

FIG. 12. Changes of the thermal pain threshold in experiments with patch application immediately after surgical incision of hind paws. The empty (framed) columns (baseline) represent the control measurement before surgery. The light gray columns show the data measured in the intact, non-operated leg, in the left group for the control patch, in the right group for the capsaicin patch. The dark columns show the data measured in the operated leg, in the left group for the control patch (patch without active ingredient), in the right group for the capsaicin patch. The surgical intervention decreased the thermal pain threshold compared to the baseline value measured in animals treated with control patches, as well as compared to the contralateral intact paw. The thermal pain threshold of the operated paw in capsaicin-treated rats was still lower than the respective baseline value, but it was significantly larger than the threshold of the operated legs of control animals with a patch without active ingredient. The thermal sensitivity of intact paws of capsaicin-treated rats did not differ from the baseline value taken before surgery. The patch without active ingredient had no effect on the thermal pain threshold of the hind paws.

FIG. 13. Changes of thermal pain thresholds in experiments with delayed patch application (after 18 hours).

The empty (framed) columns (baseline) represent the control measurement before surgery. The light gray columns show the data measured in the intact, non-operated leg, in the left group for the control patch, in the right group for the capsaicin patch. The dark columns show the data measured in the operated leg, in the left group for the control patch (patch without active ingredient), in the right group for the capsaicin patch.

Surgical incision of the paws significantly reduced the pain threshold compared to the contralateral intact paws and respective baseline values. Control patches without capsaicin (tape only) failed to improve this condition.

The capsaicin-releasing patches elevated the thermal pain threshold compared to the control patch. The elevated threshold was still lower than the baseline, no-surgery control value. Neither the control nor the capsaicin-containing patches changed the thermal sensitivity of intact paws.

FIG. 14. Capsaicin-containing patches mitigate carrageenan-induced mechanical allodynia.

The empty (framed) columns (baseline) represent the control measurement before surgery. The light gray columns show the data measured in the intact leg treated with physiological saline, in the left group for the control patch, in the right group for the capsaicin patch. The dark columns show the data measured in the leg treated with carrageenan, in the left group for the control patch (patch without active ingredient), in the right group for the capsaicin patch.

Carrageenan reduced the mechanical pain threshold detected 18 hours after the intervention in rats treated with control and capsaicin-containing patches compared to the contralateral paw. The mechanical threshold values of the carrageenan-treated paws were still reduced compared to the contralateral paws after 6 hours of treatment with capsaicin patches or their control. The mechanical pain threshold of the carrageenan-injected paws was significantly elevated by the capsaicin treatment compared to the value detected before the application of the patch. The contralateral paws injected with saline did not show mechanical allodynia.

FIG. 15. Active ingredient release curve of the patch according to Example 1.

FIG. 16. Active ingredient release curve of the patch according to Example 2.

FIG. 17. Active ingredient release curve of the patch according to Example 3.

FIG. 18. Active ingredient release curve of the patch according to Example 4.

FIG. 19. Active ingredient release curve of the patch according to Example 5.

FIG. 20. Active ingredient release curve of the patch according to Example 6.

FIG. 21. Dissolution test of the preparation according to Example 11 at 37° C.

FIG. 22. Dissolution test result of the preparation according to Example 12 at 37° C.

FIG. 23. Dissolution test of the preparation according to Example 13 at 37° C.

FIG. 24. Dissolution test of the preparation according to Example 14 at 37° C.

FIG. 25. Dissolution test of the preparation according to Example 17.

FIG. 26. The 3D molecular structure of capsaicin. Capsaicin is a natural pungent substance found in chili peppers. Capsaicin is used topically to treat different types of pain.

FIG. 27. The 3D molecular structure of diclofenac. Diclofenac is the nonsteroidal anti-inflammatory drug (NSAID) most commonly used in postoperative analgesia.

FIG. 28. Matrix-controlled transdermal patch system.

FIG. 29. Schematic figures of the incised and sutured hind paw of rats (A), the patch applied to the back of rats (B), and the measurement of the rat's thermonociceptive threshold using a water bath (C).

FIG. 30. Flow chart of the experimental protocol of Example 18.

FIG. 31. In Example 18, the effect of incisions and patches in each treatment group using a one-way ANOVA test. Treatment groups: (A) naive (no patch), (B) control patch (blind, patch without active ingredient), (C) capsaicin-containing patch, (D) diclofenac-containing patch, and (E) capsaicin-diclofenac combination patch.

FIG. 32. The results of carrageenan-induced paw inflammation model (Example 18). The effect of treatment with carrageenan and patches using a one-way ANOVA test. Treatment groups: (A) control patch (blind, without active ingredient), (B) capsaicin-containing patch, (C) diclofenac-containing patch, and (D) capsaicin-diclofenac combination patch. FIG. 32E shows the effect of the different treatments side by side.

FIG. 33. Flowchart summarizing the experimental procedures of the chronic neuropathic pain model induced by partial sciatic nerve ligation.

FIG. 34. Effects of patches containing different active ingredients on mechanical hyperalgesia at different time points (Day 7, 14 and 21) in the chronic neuropathic pain model (see Example 19) (n=7-8/group; **p<0.01, ***p<0.001, one-way ANOVA+Tukey's post hoc test).

### DETAILED DESCRIPTION OF THE INVENTION

In one aspect, the invention relates to a transdermal preparation for releasing an active ingredient with near zero-order kinetics, the matrix of which comprises at most one layer comprising the active ingredient in the form of a pure substance, preferably in a solid form (layer comprising a solid active ingredient), preferably a silicone layer, and optionally at most one additional layer that does not comprise the active ingredient in the form of a pure substance, preferably does not comprise the active ingredient in a solid form, particularly preferably a layer that is not provided with the active ingredient during production, preferably a silicone layer. The layer comprising the solid active ingredient also comprises the active ingredient in the form of a pure substance, preferably in the form of solid particles (powder). In the layer comprising the solid active ingredient, the active ingredient present in dissolved form is present in a saturated concentration. The saturation concentration can be adjusted to the desired level with an additive.

We have realized that with the preparation according to the invention, a uniform release of the active ingredient with near zero-order kinetics can be achieved.

We have also recognized that if the active ingredient is incorporated dissolved in a liquid excipient in the silicone matrix, in which the active ingredient reaches the saturation concentration, and if the silicone matrix also comprises the active ingredient in the form of a pure substance, preferably in a solid form, in a saturation concentration adjusted with a liquid additive—depending on the properties of the active ingredient used —, then the release of the active ingredient for an extended period of time with near zero-order kinetics can be realized without having to create a wavy interface between the layers or applying the active ingredient in increasing concentrations in each layer or requiring a membrane to achieve uniform, near zero-order kinetics.

The preparation according to the invention and the patch comprising it have a controlled release of the active ingredient, they are suitable for achieving near zero-order kinetics, the patch is flexible, while the dosing can be achieved by cutting to size.

In a preferred embodiment, the matrix layer comprising the solid active ingredient in the patch according to the invention comprises one or more active ingredient(s).

Preferably, another silicone matrix layer is optionally formed on this matrix layer, which does comprise liquid excipient, but does not comprise the active ingredient, and optionally/preferably, a pressure-sensitive adhesive silicone tape layer comprising liquid and/or solid excipient is formed.

Preferably, the additional silicone layer layered on the layer comprising the solid active ingredient comprises liquid and/or solid excipient, but does not comprise the active ingredient, and the attachment of the silicone matrix consisting of one or two layers to the skin is ensured by a pressure-sensitive adhesive silicone tape layer formed on the surface of the matrix, which optionally comprises liquid and/or solid excipients. If the additional silicone layer comprises an active ingredient, it is not present in the form of a pure substance, preferably not present in a solid form; optionally it enters this layer by diffusion, or optionally it enters this layer during production, but preferably its concentration is lower than the saturation concentration and/or than the concentration of the active ingredient in the layer comprising the solid active ingredient.

Preferably, according to the invention, the matrix layer is cross-linked polydimethylsiloxane rubber with apolar properties, and the active ingredient is mixed in it. In particular, the active ingredient is not mixed in the adhesive layer of the patch.

The active ingredient is preferably dissolved in the liquid excipient(s) as solvent(s), which are not removed from the matrix of the patch.

In the patch according to the invention, the mixture comprising the solution of the active ingredient, preferably comprising a polar liquid and optionally a non-polar liquid, remains in the silicone matrix and is able to diffuse there.

In the solution according to the invention, the solid active ingredient is preferably present in crystalline form.

In general, the solid active ingredient can be in amorphous and/or powder-like form.

The inventors surprisingly found that if the active ingredient is dissolved in a polar liquid, in a mixture of polar liquids or a mixture of polar and apolar liquids, they can create saturated solutions with different concentrations of the active ingredient.

The characteristic of our invention is that the active ingredient dissolved in the liquid excipient at a saturation concentration is dispersed in the silicone matrix and comes into contact with the particles of the active ingredient present in solid form. In this way, the solid active ingredient is continuously dissolved in place of the active ingredient that is released and leaves from the layer during use, thus the saturation concentration of the active ingredient is ensured. The thickness of the layer is decisive for the size of the particles. Preferably, the size (diameter) of the particles is 5 to 200 microns, preferably 20 to 150 microns, particularly preferably 20 to 125 microns. The surface-to-volume ratio can be important here, as the dissolution must be sufficiently fast to replace the active ingredient that is leaving.

In a further aspect, the invention relates to a low-dose patch comprising capsaicin or a capsaicin analogue as an active ingredient, which provides the capsaicin or capsaicin analogue uniformly with near zero-order kinetics.

In a preferred embodiment, the amount of the active ingredient that dissolves (diffuses) into the second layer that does not comprise the solid active ingredient, but comprises liquid excipient and/or the amount of the active ingredient dissolved into the liquid excipient comprised in the pressure-sensitive adhesive layer helps to ensure that the given active ingredient is delivered into the skin and then into the body in an appropriate, regulated (optionally predetermined) amount. The embodiment of the transdermal preparation according to the invention ensures the uniform release of the active ingredient with near zero-order kinetics. Preferably, the layer of the preparation in contact with the skin surface is a pressure-sensitive adhesive layer (PSA), which ensures the attachment of the preparation to the skin. The transdermal patch according to the invention ensures the zero-order kinetic release of the active ingredient without the need for the layers of the silicone matrix to comprise increasing concentrations of the active ingredient the farther away they are from the skin, i.e. to have to compensate the increasing distance by an increasing concentration gradient, and there is no need for the matrix layers to contact with a wavy surface, i.e. with an increased contact surface, in order to achieve zero-order kinetics of active ingredient release. There is no need either for other regulators to achieve near zero-order kinetics, e.g. regulatory membrane layers.

The matrix of the transdermal patch consists of one or optionally two layers, which is considered to be a preparation according to the invention. The raw material of the matrix formed in the form of a layer is preferably silicone, which has many advantageous properties. For example, silicone is a skin-friendly material. In addition, silicone and its properties can be modified by adding various excipients, which allows the matrix to be “tailored” to the given active ingredient, and designed accordingly. Furthermore, the transdermal patch comprising the silicone matrix can be freely cut to size, since the silicone keeps within itself the solvent comprising the active ingredient, the active ingredient does not “flow out” during cutting; thus, its application is much safer than previous solutions. (The solvent comprising the active ingredient is the liquid excipient.)

In one aspect, the active ingredient is a capsaicinoid, preferably capsaicin or a capsaicin analogue.

In a further variant of the invention, the active ingredient is a small-molecule organic active ingredient whose solubility in a silicone matrix can be controlled with an apolar or polar solvent.

Preferably, the active ingredient is a small organic molecule soluble in monohydric or polyhydric alcohols, particularly dihydric or polyhydric alcohols, for example dihydric or trihydric alcohols.

In a preferred embodiment, the active ingredient is selected from the following:
non-steroidal anti-inflammatory drugs (NSAIDs), preferably diclofenac.
NSAIDs include, among others:
salicylic acid derivatives, such as aspirin and its derivatives,
propionic acid derivatives, such as naproxen or ibuprofen,
acetic acid derivatives, such as indomethacin, diclofenac,
pyrazolone derivatives, such as phenylbutazone and its derivatives or aminophenazone and its derivatives, aniline derivatives, such as acetanilide, phenacetin and paracetamol and their derivatives,
anthranilic acid derivatives, such as glafenin, flufenamic acid, fenamate, mefenamic acid, niflumic acid.

Particularly preferably, the active ingredient is present in the matrix layer of the patch according to the invention in the proportion specified in the brief description of the invention. Particularly preferably, it is present in solution in a proportion of at most 0.5% by weight, preferably in a proportion of at most 0.4% by weight, particularly preferably in a proportion of at most 0.3% by weight. Preferably, this is the concentration of the solution, preferably its saturation concentration.

Particularly preferably, in the patch according to the invention, the active ingredient is present, as a measured concentration, in a surface ratio of at most 1 mg/cm², preferably 0.5 mg/cm², more preferably 0.4 mg/cm², particularly preferably 0.3 mg/cm², with reference to the surface of the patch in contact with the skin.

In a preferred embodiment, capsaicin is present in the patch at a concentration of at most 5 mg/g, based on the matrix layer comprising the active ingredient in solid form or based on the matrix layer and the regulator layer. It is preferably present in a concentration of at most 2.3 mg/g, particularly preferably in a concentration of 1 mg/g to 2.3 mg/g, and in a preferred variant, in a concentration of at most 1 mg/g.

In a preferred embodiment, the active ingredient is selected from the following:
nitrites and nitrates, nitrate esters, sugar esters or esters of sugar derivatives, preferably nitrate esters of sugars and sugar derivatives, nitrate esters of heterocyclic compounds, preferably nitrate esters of hydroxy-substituted heterocyclic compounds, preferably nitrates of heterocycles with O-containing ring, preferably nitrate esters of hydroxy-substituted O-containing heterocycles.

Particularly preferably, it is isosorbide dinitrate (ISDN).

Preferably, the nitrate and nitrate ester compounds are drugs for cardiovascular diseases, such as blood pressure regulators, drugs affecting the heart, preferably drugs against heart failure, muscle relaxants, chest pain relievers, etc.

Preferably, the thickness of the silicone matrix layers of the transdermal preparation is 0.2 mm to 0.6 mm.

Preferably, the active ingredient present in the form of a pure substance is in a solid state.

Particularly preferably, the amount of the active ingredient present in the form of a pure substance is 1% to 5%.

Preferably, the amount of liquid excipient saturated with the active ingredient in the layer is 1% to 15%. Particularly preferably, the silicone layers comprise 1% to 15% of liquid and 0% to 5% of solid excipient.

In a further preferred embodiment of the invention, the raw material of the silicone layer is a crosslinked polydimethylsiloxane comprising reactive groups or a mixture of polydimethylsiloxanes.

A further preferred embodiment is if the layer comprising the active ingredient comprises a solid excipient, preferably colloidal silica, and/or carbonate in powder form, preferably calcium carbonate and/or solid carbohydrate in powder form for dispersing the active ingredient in the form of a pure substance, preferably a solid active ingredient (and optionally also in the form of a liquid saturated solution).

In a preferred embodiment, the liquid excipient is an apolar liquid, preferably selected from the group consisting of silicone oil, cyclic methylsiloxane and/or a mixture thereof.

In a further preferred embodiment, the liquid excipient is a polar liquid, preferably selected from the group consisting of monohydric, dihydric or trihydric alcohols, polyalcohols, polyethers and/or mixtures thereof.

Preferably, the liquid excipient is a polar liquid, preferably an alcohol, particularly—if used—a non-volatile compound of a monohydric alcohol. If a volatile solvent is used during the production of the patch, it is allowed to evaporate during the preparation of the patch so that the dissolution and solubility conditions in the finished patch do not change.

Particularly preferably, the polar liquid is selected from dihydric or trihydric alcohols, polyalcohols, particularly preferably it is glycerol.

In a further preferred embodiment, the liquid excipient is a mixture of apolar and polar liquids.

Another preferred embodiment is if the homogeneous distribution of the liquid excipients in the silicone matrix is facilitated by the use of anionic and/or nonionic surfactants.

Particularly preferably, the surfactant is a non-ionic surfactant, preferably a sorbitol derivative, preferably an ethoxylated sorbitan esterified with fatty acids (polysorbate). Some example:
Polysorbate 20 (polyoxyethylene (20) sorbitan monolaurate)
Polysorbate 40 (polyoxyethylene (20) sorbitan monopalmitate)
Polysorbate 60 (polyoxyethylene (20) sorbitan monostearate)
Polysorbate 80 (polyoxyethylene (20) sorbitan monooleate).

Another preferred embodiment is that a pressure-sensitive adhesive silicone layer comprising 0% to 7% of liquid excipients and/or 0% to 5% of solid excipients is formed on the surface of the silicone layer or layers, which ensures attachment to the skin.

Particularly preferably, the thickness of the pressure-sensitive adhesive layer is 0.1 mm to 0.5 mm.

In a preferred embodiment, the invention relates to a transdermal patch, which, when placed on the skin, ensures the release of the active ingredient it contains over a long period of time, in particular for 4 to 24 hours, with near zero-order kinetics, characterized in that the preparation consists of one or two silicone layer(s), which have a layer thickness of 0.1 mm to 1 mm, preferably 0.2 mm to 1 mm or 0.1 mm to 0.8 mm, in particular 0.2 mm to 0.6 mm, and one of which comprises the active ingredient in the form of a saturated solution prepared with a liquid excipient, as well as in the form of a pure substance, preferably in a solid form, while—if this is necessary—the second silicone layer layered on top of, which has a layer thickness of 0.1 mm to 1 mm, preferably 0.2 mm to 1 mm or 0.1 mm to 0.8 mm, in particular 0.2 mm to 0.6 mm, does not comprise an active ingredient, only liquid and/or solid excipients. The attachment of the silicone matrix consisting of one or two layer(s) to the skin is ensured by a pressure-sensitive adhesive silicone tape layer, preferably 0.1 mm to 0.5 mm thick and comprising liquid and/or solid excipients, formed on the surface of the matrix.

Our invention also relates to a method for the production of a transdermal preparation that releases its active ingredient over a longer period of time, preferably for at least 4 hours, more preferably for 6 hours, particularly preferably for 4 to 24 hours, particularly for 6 to 12 hours, with near zero-order kinetics, characterized in that the active ingredient is mixed into the silicone raw material of the preparation in the form of a saturated solution prepared with a liquid excipient, as well as in the form of a pure substance, and a solid excipient is also added to the mixture, then it is spread on a carrier film, preferably in a layer thickness of 0.2 mm to 0.6 mm, and cross-linked, then it is covered with a pressure-sensitive adhesive layer comprising liquid and/or solid excipients.

Particularly preferably, the crosslinker also comprises a catalyst, which comprises a crosslinking agent and a compound that catalyzes crosslinking. Particularly preferably, the catalyst is an oxalaminidate-based catalyst, in particular Oxam crosslinking catalyst.

In a variant, the catalyst is added separately to the crosslinking agent. In a preferred embodiment, the crosslinker is an alkoxysilane crosslinker, for example a tetraethoxysilane crosslinker. Preferably TES-40.

Such cross-linking is described e.g. in Hungarian patent no. HU207344.

In the method according to the invention, preferably, the amount of the liquid excipient saturated with the active ingredient is set to 1% to 15%, the amount of the active ingredient present in the form of a pure substance is set to 1% to 5%, so that the solubility of the active ingredient is at a desired level to achieve saturation.

Another preferred embodiment is to also mix 0% to 5% of solid excipient into the matrix, preferably by powder dilution (trituration) in order to evenly distribute the solid active ingredient. Preferably, the particle size defined herein also applies to the solid excipient.

Another preferred embodiment is that the silicone mixture comprising the active ingredient and excipients is spread out and cross-linked in a layer thickness of 0.2 mm to 0.6 mm.

A preferred embodiment of the method according to the invention is to layer a silicone layer comprising no active ingredient but comprising 1% to 15% of liquid and 0% to 5% of solid excipients, with a layer thickness of 0.2 mm to 0.6 mm, onto the matrix comprising the active ingredient.

Another preferred embodiment is that a pressure-sensitive adhesive silicone layer comprising 0% to 7% of liquid and 0% to 5% of solid excipient is formed on the surface of the silicone layer or layers, which ensures attachment to the skin.

Another preferred embodiment is to use polydimethylsiloxanes comprising reactive groups and cross-linking by condensation and/or addition method as the raw material of the matrix layers.

Another preferred embodiment is if, depending on the properties of the active ingredient, apolar liquids, preferably silicone oils, cyclic methyl siloxanes, and/or polar liquids, preferably monohydric, dihydric or trihydric alcohols, polyalcohols, polyethers and/or a mixture thereof are used as a liquid excipient.

Another preferred embodiment is if the homogeneous distribution of the liquid excipients in the silicone matrix is facilitated by the use of anionic and/or nonionic surfactants.

Another preferred embodiment is if the distribution of the active ingredient in the form of a pure substance or a liquid saturated solution is facilitated by the use of a solid excipient, preferably colloidal silica, solid carbohydrates in powder form.

In a preferred embodiment of the invention, the basis of the silicone polymers is dimethylpolysiloxane (PDMS), a linear silicone polymer, which turns into an elastic silicone rubber when cross-linked. PDMS can be used as a support matrix. There are two main conventional methods to crosslink PDMS: condensation and addition.

In polymers produced by condensation, Si—O—Si bonds are the crosslinks. In the case of the addition technique, Si—C—C—Si bonds provide the crosslinks between the PDMS chains. The use of condensation polymers for medical purposes is complicated by the fact that achieving the purity required in healthcare is a difficult and expensive process. Another problem is that the process of polycondensation (FIG. 2) needs a tin compound catalyst, which raises problems in terms of biocompatibility.

The cross-linked structure of addition polymers is generally more suitable for medical use (FIG. 3). The final matrix structure can be transformed by different additives. This can improve the admixing of the active ingredient and the matrix. The addition silicone polymer can be prepared from two main components: linear PDMS containing vinyl groups and the crosslinker phase that contains a hydrosilane compound and a platinum compound, these materials are biocompatible. The active ingredient and adjuvants are incorporated into the polymer of the TTS [Guedes, V. et al., 2018][5]. The viscous mixture cures in about 30-60 minutes at a temperature of 70° C., depending on the composition. Due to the poor mechanical properties of the silicone polymer, a carrier layer is required. Usually, a metal or polymer film is widely utilized. The polymer mixture can be applied to the carrier layer by a single-layer spreading method (FIG. 4).

In an embodiment, the addition silicone is the so-called Elastosil. Its chains comprise only methyl groups, and sometimes vinyl groups in the middle of the chain and at the ends of the chains. The other component is a short siloxane compound comprising H—Si end groups. A hydrosilylation reaction takes place between the vinyl and H-silanes under the influence of a platinum catalyst, thus cross-linking the silicone. This raw material has no polar groups, the matrix is completely apolar. Therefore, it is advisable to use surfactants (tensides) in order to disperse the polar excipient.

Another preferred siloxane variant is polydimethylsiloxane-α,ω-diol. This solution is preferred, because during cross-linking some of the polyols chemically “bind” into the cross-linked structure, thus increasing its polarity. Afterwards, the polar excipients are also more evenly distributed in the cross-linked network, since hydrogen bridges can form between the molecules of the excipient and the siloxane chains (now also comprising polar groups).

The present preparation consists of one or two silicone elastomer layer(s) cross-linked by addition and/or condensation method, as well as a pressure-sensitive silicone layer for attaching the layers to the skin, and its essence is that the elastomer layer present under the silicone adhesive layer comprises the active ingredient in a saturation concentration.

The low-dose (less than 1%) capsaicin used in our invention causes a long-lasting, low-intensity activation of the TRPV1 receptor, which triggers the release of locally vasodilating, skin-reddening substances (calcitonin gene-related peptide (CGRP)), the blood supply to the inflamed area is increased, and this promotes the absorption of other pain-relieving substances, e.g. non-steroidal anti-inflammatory drugs, etc.

We realized that if a low dose of capsaicin is placed in a transdermal patch (transdermal therapeutic system, TTS) that releases it evenly over a long period of time, thereby being able to deliver it into the deeper layers of the skin, a pain-relieving effect is created. This is due to somatostatin released from the sensory nerve endings. In addition, as a result of long-term, low-intensity activation of capsaicin-sensitive sensory nerve endings, local blood flow increases, which process can facilitate the absorption of other active ingredients (e.g. non-steroidal anti-inflammatory drugs, NSAIDs). The inventors realized that the co-formulation of capsaicin and an NSAID active ingredient (e.g. diclofenac) in one TTS has beneficial properties.

In a preferred embodiment, the capsaicin is present in the patch at a concentration of at most 5 mg/g, based on the matrix layer comprising the active ingredient in solid form or based on the matrix layer and the regulator layer. It is preferably present in a concentration of at most 2.3 mg/g, particularly preferably in a concentration of 1 mg/g to 2.3 mg/g, and in a preferred variant, in a concentration of at most 1 mg/g.

Particularly preferably, the patch according to the invention comprises one or more active ingredient(s).

Particularly preferably, in the patch comprising capsaicin and diclofenac, diclofenac is present in a proportion of at most 0.5% by weight, preferably 0.4% by weight.

Particularly preferably, in the patch comprising capsaicin and diclofenac, the capsaicin is present in a surface ratio of at most 0.5 mg/cm², preferably 0.4 mg/cm², as a measured concentration, with reference to the surface of the patch in contact with the skin.

Particularly preferably, in the patch comprising capsaicin and diclofenac, the diclofenac is present in a surface ratio of at most 0.5 mg/cm², preferably 0.4 mg/cm², as a measured concentration, with reference to the surface of the patch in contact with the skin.

The inventors unexpectedly found that the presence of capsaicin in the patch prolongs the effect of diclofenac. Preferably, the effect of diclofenac is present even after 4 hours, particularly preferably even after 6 hours.

In a further aspect, the invention relates to a transdermal patch, which, when placed on the skin, ensures the release of the low-dose capsaicin active ingredient it contains over a long period of time, in particular for 4 to 24 hours (preferably for 6 to 12 hours) with near zero-order kinetics. The preparation consists of one or two, preferably one-component and/or two-component silicone elastomer layer(s), the layer thickness of which is in particular 0.1 mm to 0.5 mm.

The capsaicinoid-content, e.g. capsaicin-content or capsaicin analogue-content, of the transdermal patch (preferably the combined amount of dissolved capsaicin and solid capsaicin based on the volume of the layer comprising the (solid) active ingredient) is at most 1%, preferably 0.05% to 1.0%. Preferably it comprises the capsaicin in a saturation concentration of at most 0.5%, more preferably less than 0.4%, preferably at most 0.3%.

More preferably, the preparation comprises capsaicin or a capsaicin analogue in a saturation concentration of 0.05% to 0.5%, particularly 0.1% to 0.4%, 0.05% to 0.3%. In a particularly preferred variant, the preparation comprises capsaicin in a saturation concentration of more than 0.1% and less than 0.35%, preferably 0.15% to 0.3%, particularly 0.2% to 0.3%.

In the solution according to this aspect of the invention, capsaicinoids can also be used.

Capsaicinoids are amides of vanillylamine (I) amidated with C₇-C₁₃, particularly C₉-C₁₃ or C₇-C₁₁, preferably C₉-C₁₁ fatty acids, which can be isolated from nature or can be artificial, and their effect is similar to that of capsaicin, preferably acting on the TRPV1 receptor. Capsaicinoids are described, for example, by Kaiser, M. et al [Kaiser, M. et al., 2017, p. 500]; such compounds are, for example, the following: capsaicin, dihydrocapsaicin, norhydrocapsaicin, homodihydrocapsaicin, homocapsaicin, nonivamide, omega-hydroxycapsaicin.

An example of artificial capsaicinoid is zucapsaicin (Z-capsaicin), which is the cis-isomer of capsaicin.

Typically, the effect of capsaicinoids on the Scoville scale is between 1000 or 3000 and 30000, preferably between 2000 or 5000 and 25000, particularly preferably between 5000 and 20000.

In general, capsaicinoids have the following formula

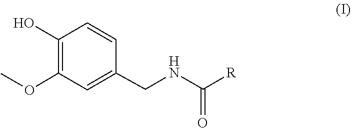

wherein R is substituted or unsubstituted C₇-C₁₃, particularly C₉-C₁₃ or C₇-C₁₁, preferably C₉-C₁₁ alkyl or alkenyl, which, if substituted, is substituted with at most 1 to 3, preferably 1 to 2, substituents selected from the group consisting of ethyl, methyl, halogen, hydroxy, preferably ethyl, methyl and hydroxy, particularly preferably methyl and hydroxy. Preferably, the alkenyl comprises 1 or 2, preferably a single double bond.

Particularly preferably, the substituted or unsubstituted C₉-C₁₁ alkyl is selected from the group consisting of:

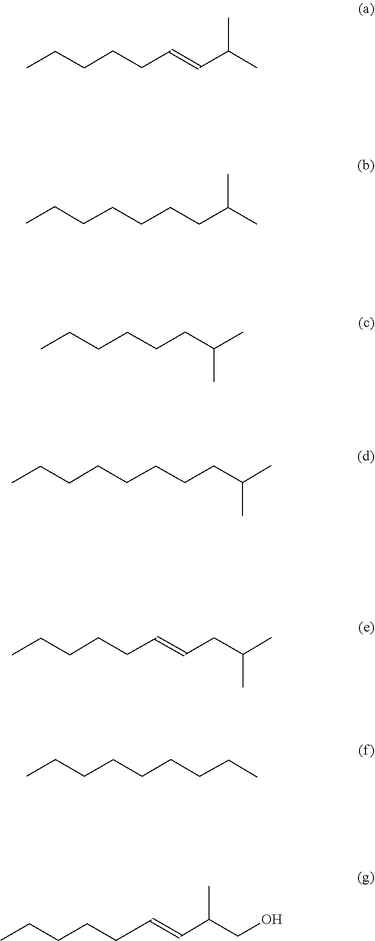

A detailed overview of capsaicinoids, methods for their measurement and testing can be found in the doctoral thesis of Dr. Mónika Kuzma (Kuzma, M 2016), the content of which is considered part of the teaching in this regard, and the capsaicinoids described therein are claimed for the implementation of the invention.

In the solution according to this aspect of the invention, capsaicin analogues can also be used.

Capsaicin analogues are well known in the art. The pain-relieving effect of capsaicin analogues have already been described, for example, by Hayes A. G. et al. in their article in 1984 [Hayes, A G et al., 1984].

The structure-function relationship of such is described, for example, in the following publication: [Huang X F et al., 2013]

Huang X F, Xue J Y, Jiang A Q, Zhu H L. Capsaicin and its analogues: structure-activity relationship study. Curr Med Chem. 2013; 20(21):2661-72. doi: 10.2174/0929867311320210004. PMID: 23627937.

The use of capsaicin analogues in patches is described, e.g. in U.S. Pat. No. 8,821,920B2.

The total synthesis of capsaicin analogues is described by Anderson, Mattias et al. [Anderson, M et al., 2014].

Anderson, Matthias; Afewerki, Samson; Berglund, Law; Córdova Armando. Total Synthesis of Capsaicin Analogues from Lignin-Derived Compounds by Combined Heterogeneous Metal, Organocatalytic and Enzymatic Cascades in One Pot. Advanced Synthesis and Catalysis 2014, 356(9), 2113-2118 https://doi.org/10.1002/adsc.201301148

Preferably, the attachment of the patch to the skin is ensured by a pressure-sensitive silicone adhesive layer layered onto the silicone layer(s), in particular 0.1 mm to 0.5 mm thick.

In a preferred variant, the invention also relates to a method for producing these transdermal patches, characterized in that the raw material of the patch is a silicone oligomer cross-linking by addition and/or condensation method, which forms one or two layer(s) that are layered on each other, preferably with a layer thickness of 0.1 mm to 0.6 mm, and cross-linked, and their attachment to the skin is ensured by a pressure-sensitive silicone adhesive layer formed on their surface, preferably with a layer thickness of 0.2 mm to 0.5 mm. A preferred embodiment of the method according to the invention is if the silicone layer located farthest from the skin comprises the capsaicinoid active ingredient, preferably capsaicin, in 0.1% to 1%, preferably in a proportion greater than 0.1% (1 mg/ml) and smaller than 1% (10 mg/ml). Particularly preferably, the silicone layer comprising the (solid) active ingredient comprises the capsaicinoid, preferably capsaicin in 0.15% to 0.8%, preferably 0.15% to 0.6%, particularly preferably 0.15% to 4%, preferably 0.15% to 3.5%, particularly preferably in 2% to 3%.

The solution phase of the silicone layer comprises the active ingredient in a saturation concentration during the period of stable active ingredient release. Another preferred embodiment of the invention is if the liquid and/or solid excipients facilitating the diffusion of capsaicin in the matrix are present in the silicone matrix in 4% to 20%. In a preferred embodiment, the silicone oligomer is a silicone elastomer.

The preparations according to the invention are suitable for treating not only humans, but also other mammals.

When placed on the patient's skin, the transdermal preparation according to the invention can ensure the delivery of the active ingredient into the skin at a uniform rate for 4 to 24 hours, preferably for 6 to 12 hours, in the form of patches. In the case of a preparation with a given composition, the desired dose can be achieved by using a patch of the appropriate size.

The advantage of using low-dose capsaicin patches with zero-order kinetics compared to high-dose patches is that their effect only lasts as long as the patch is on the skin, while high-dose patches desensitize the given skin area for a long time, and the loss of pain sensation can have serious consequences.

The transdermal patch according to the invention comprises a low dose of capsaicin (at most 1%), consists of one or two silicone layer(s), with a pressure-sensitive adhesive layer on the skin-contacting surface of the silicone layer(s). In an embodiment, the patch consists of a silicone layer comprising a low dose of capsaicin and a pressure-sensitive adhesive layer. In another embodiment, the patch consists of two silicone layers and a pressure-sensitive adhesive layer, wherein only one of the two silicone layers, the layer farthest from the skin, comprises the solid active ingredient (e.g., low-dose capsaicin); preferably, no active ingredient is added to the other silicone layer during production (manufacture), but the active ingredient is transferred to this layer by diffusion.

In a preferred embodiment, the silicone layers comprise capsaicin and/or its analogues in 0.1% to 1.0%, preferably in any of the concentration ranges specified herein.

In a preferred embodiment, the transdermal patch or the preparation in the patch comprises 4% to 20% liquid and/or solid excipient. In a preferred embodiment, the liquid excipient is selected from monohydric and/or polyhydric alcohols and surfactants. In a preferred embodiment, the solid excipient is an active solid excipient, preferably amorphous colloidal silica; and/or an inactive solid excipient, preferably calcium carbonate.

In a preferred embodiment, the raw material of the silicone layers is a silicone cross-linking by condensation and/or addition method.

In a preferred embodiment, the thickness of the individual silicone layers and the adhesive layer is 0.1 mm to 0.5 mm each.

Unexpectedly, we have realized that if a low dose of capsaicin and/or its analogues (hereinafter: capsaicin) is placed in a transdermal patch that can deliver it uniformly, over a long period of time, into the deeper layers of the skin, a pain-relieving effect can be observed, i.e. the low-dose capsaicin and/or its analogues, which are compounds of natural plant origin, penetrate the skin and increase the release of pain-relieving, anti-inflammatory substances, thus activating the endogenous pain-relieving system.

Another significant realization of our invention is that the transdermal patch can be produced in such a way that a low dose of capsaicin is placed in a silicone matrix raw material and the matrix also comprises liquid and/or solid excipients that promote the diffusion of capsaicin within the matrix, and then this layer is cross-linked in a layer thickness of 0.1 mm to 0.6 mm. Then the release of capsaicin from the matrix can follow the zero-order release kinetics. We have found that capsaicin in the matrix must be present in an amount greater than its solubility in the components, so that the amount released from the matrix can be continuously replenished. The zero-order release kinetics can also be achieved by layering an additional layer comprising no active ingredient, only liquid and/or solid excipients onto the matrix comprising the active ingredient with a layer thickness of 0.1 mm to 0.5 mm, in which the solubility of capsaicin is lower than that of the matrix comprising the active ingredient, and then cross-linking this layer, as well. Thus, the second layer (which is the layer closer to the skin during use) is permanently saturated—regarding capsaicin —, so the release of the active ingredient from it approaches zero-order kinetics. Whether a matrix consisting of one or two layer(s) is prepared, a thin, in particular with a layer thickness of 0.1 mm to 0.5 mm, pressure-sensitive silicone adhesive layer is layered onto it, which ensures that the patch is attached to the skin with appropriate strength.

The advantage of the transdermal patch according to the invention is that, when placed on the patient's skin, it can ensure the delivery of the active ingredient into the skin at a uniform rate for a long period of time, preferably for 4 to 24 hours, particularly preferably for 6 to 12 hours, in the form of patches of different sizes. In the case of a preparation with a given composition, the desired dose can be achieved by using a patch of the appropriate size, the patch can be cut to size. A significant advantage of using low-dose capsaicin patches with zero-order kinetics compared to high-dose patches is that they are able to deliver the capsaicin into the deeper layers of the skin over a long period of time, and as a result, a pain-relieving effect can be observed. Their significant advantage is that their effect only lasts as long as the patch is on the skin, while high-dose patches desensitize the given skin area for a long time, and the loss of pain sensation can have serious consequences.

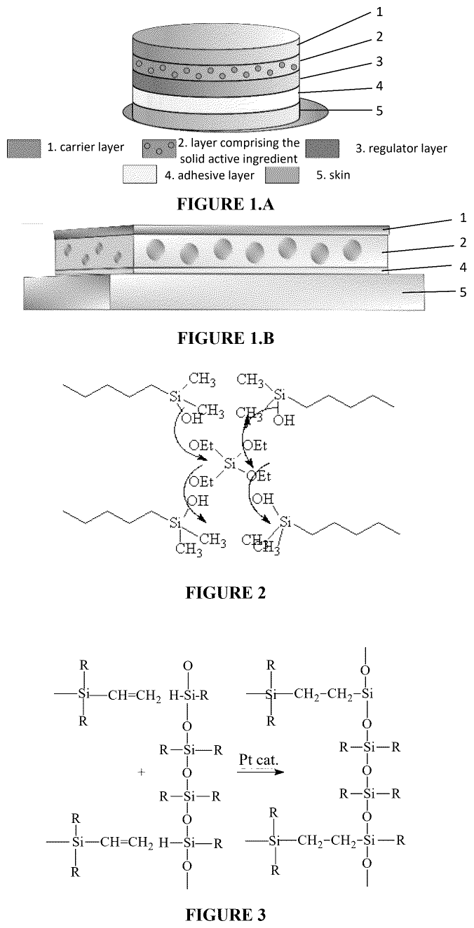

Among transdermal therapeutic systems (TTS), modified silicone-polymer-based techniques provide a well-controlled and cost-effective matrix diffusion system. We have developed and investigated capsaicin-containing transdermal patches made by this procedure (FIG. 1).

In the matrix system, the drug is embedded in a polymer layer, which is covered by a drug-free regulator layer with different diffusion properties. A controlled drug release profile can be achieved by precisely adjusting the gradient based on the different diffusion constants in the two layers.

According to our experience, low-dose (less than 1%) capsaicin administered via the patch according to the invention causes a long-lasting, low-intensity activation of the receptor, which triggers the release of locally vasodilating, skin-reddening substances (calcinonin gene-related peptide), the blood supply to the inflamed area is increased, and this promotes the absorption of other pain-relieving substances (e.g. non-steroidal anti-inflammatory drugs, etc.).

The innovative structure of the TTS according to the invention, the silicone polymer matrix provides a suitable carrier material for small-molecule, organic active ingredients (e.g. capsaicinoids, allyl sulfides) that have significant therapeutic potential in the development of new types of analgesic and anti-inflammatory agents.

Thanks to this structure, the TTS comprising a low dose of capsaicin ensures the delivery of the active ingredient into the skin at a uniform rate over a long period of time.

TTS comprising a low dose of capsaicin can be used to (1) provide a steady rate, long-lasting release of the active ingredient; (2) the active ingredient can be delivered into the deeper layers of the skin, as well; (3) the therapeutic effect can be terminated at any time by removing the patch; (4) by cutting the patch to size, the dose to be applied can be easily adjusted; (5) the contamination of the hands and clothing can be easily avoided, thus—in the case of pain-relieving patches—effective pain relief can be achieved without the loss of function of the sensory nerves, or without the initial pain-inducing effect.

In the TTS according to the invention, the carrier material of the low-dose capsaicin is thus a silicone polymer matrix, in which, due to the cross-linked structure, the active ingredient is evenly distributed in the patch, thus ensuring its uniform and long-lasting release. Due to the innovative carrier material and the novel type of mechanism of action of pain relieving, it has many advantages over commercially available high-dose patches or creams. It should be emphasized that these benefits are maintained even when low-dose capsaicin is combined with other active ingredient.

Another surprising discovery of the invention has been that in the case of the capsaicin-diclofenac combined active ingredient patch, interestingly and unexpectedly, the heat threshold of the experimental rats greatly and significantly increased already 2.5 hours after the patch was applied, thus the analgesic effect of the patch has already been detectable, and this significant pain-relieving effect remained stably observable 6 hours after the application of the patch, although in the case of the patch comprising only diclofenac this effect had already ceased after 6 hours, and in the case of the patch comprising only capsaicin it had not yet appeared.

One of the aims of the invention has been the further development of low-dose capsaicin TTS, during which capsaicin has been combined with an NSAID, diclofenac, whose solubility in a silicone matrix can be well controlled with an apolar or polar solvent. The novel combination of low-dose capsaicin and diclofenac in one patch is a unique, gap-filling product on the market of pain-relieving products, which is in great demand in neurological and rheumatological patient care. The devices and methods used to create the TTS model industrial production, thus the technologies used can be easily adapted to small-scale production with minor modifications.

One aim of the invention has been to create a transdermal therapeutic patch that, comprising both capsaicin and diclofenac, can effectively relieve various pains by inducing two mutually reinforcing pharmacological effects.

Due to the structure of the product (silicone polymer-based matrix diffusion-controlled TTS), it has a number of useful and novel properties, such as predictable, controlled drug delivery, the patch can be cut to size, thereby precisely adjusting the dose of the drug, and thus minimizing the possible side effects that occur with all drugs.

The capsaicin-containing transdermal patch and the combined capsaicin-diclofenac containing transdermal patch developed by the present inventors ensures that the drug is released uniformly from the patch over a long period of time according to a prolonged kinetics, thanks to the silicone polymer matrix carrier material [László et al., 2022]. Therefore, the treatment of chronic pain conditions is expected to be one of the main indications of the patches, and the majority of patients in chronic neurological and rheumatological care could be the primary target group for its application.

The effectiveness of the capsaicin-diclofenac combined active ingredient transdermal patch, which is not yet available on the market, has been investigated in both in vitro and in vivo systems. In addition to traditional in vitro methods (Franz cell and Raman spectroscopy), we also used innovative methods (so-called flow-through cell) to model the components of the skin during the examination of the release and absorption of the active ingredients from the patch. During the in vivo tests, on the one hand, we measured the pain response to thermal stimuli (thermal allodynia) in a rat model of postoperative pain induced by surgical incision of the plantar skin-muscle of a hind paw, using an increasing-temperature water bath, and on the other hand, we examined the pain threshold to mechanical stimuli (mechanical hyperalgesia) during acute inflammation induced by carrageenan injected into a hind paw, with a dynamic plantar aesthesiometer. This methodological toolkit is excellent for testing the effectiveness of analgesic drug candidate molecules to be used in transdermal systems.

Furthermore, based on the abovementioned beneficial properties and that the preliminary results of the acute pain models have been very promising, the inventors aimed to perform the preclinical study of the capsaicin-containing transdermal patch and the combined capsaicin-diclofenac containing transdermal patch for chronic neuropathic pain in a rat model induced by partial nerve ligation (Seltzer operation). The present inventors' experiments have demonstrated that both capsaicin-containing and combined capsaicin-diclofenac containing silicone polymer matrix-based transdermal patches are effective in relieving neuropathic pain in the rat model of chronic neuropathy induced by partial sciatic nerve ligation, thus the capsaicin-containing and the combined capsaicin-diclofenac containing patches is a promising and efficacious therapeutic tool for the treatment of this pain condition.

Preferably, the patch, preferably the matrix within it, does not comprise a penetration enhancer.

Particularly preferably, the patch does not comprise sufentanil as an active ingredient.

Particularly preferably, the patch does not comprise fentanyl as an active ingredient.

Preferably, the patch according to the invention comprises capsaicin and diclofenac. Particularly preferably, the active ingredient content of the patch according to the invention essentially consists of capsaicin and diclofenac.

Particularly preferably, the patch according to the invention comprises only capsaicin and diclofenac as active ingredients.

Particularly preferably, in the patch comprising capsaicin and diclofenac, capsaicin is present in a proportion of at most 0.4% by weight.

Particularly preferably, in the patch comprising capsaicin and diclofenac, diclofenac is present in a proportion of at most 0.4% by weight.

Particularly preferably, in the patch comprising capsaicin and diclofenac, the capsaicin is present in a surface ratio of at most 0.4 mg/cm², with reference to the surface of the patch in contact with the skin.

Particularly preferably, in the patch comprising capsaicin and diclofenac, the diclofenac is present in a surface ratio of at most 0.4 mg/cm², with reference to the surface of the patch in contact with the skin.

We can therefore say that we have prepared and tested transdermal patches that release the active ingredient capsaicin in a controlled manner, closely approximating the ideal zero-order kinetics. Our transdermal patch was a modified silicone polymer-based diffusion gradient-controlled system that provided optimal drug release and cost-effective therapy. Patches were produced using the addition-crosslinked silicone polymer method, comprising different concentrations of capsaicin, and tested under “in vitro” and “in vivo” conditions [Nalamachu, S. et al. 2020].

Addition-type silicone has very apolar properties. Since capsaicin shows relatively polar character (compared to the silicone matrix), a more polar environment had to be created in the apolar matrix in order to promote the delivery of the active ingredient in the right amount, and for this it was necessary that the molecules of the active ingredient move properly inside the silicone matrix [Wagner O., 1991]. Capsaicin is very soluble in alcohols, but monohydric alcohols with shorter carbon chains are volatile compounds and exert detrimental effects in the human skin. The simplest trivalent alcohol, glycerol, was used as a solvent. It dissolves capsaicin relatively well and is skin-friendly. However, glycerol is insoluble in silicone oligomers, therefore we used an emulsifier to achieve even distribution in the matrix [Wagner O. 1998]. Since glycerol saturated with capsaicin does not contain enough capsaicin to adequately deliver the desired amount of the drug and to ensure zero-order kinetics, solid capsaicin was dispersed in the matrix.

Capsaicin was subjected to powder dilution (triturated) with calcium carbonate to ensure precise dosing and homogeneous drug distribution. In this example, a drug-free control layer—which also contained glycerol—was applied to the drug-containing layer. Capsaicin can diffuse through this unimpeded, but only in the desired amount.

Patches containing, among others, 1 mg/g and 2.3 mg/g capsaicin were prepared and tested (see Example 8).

In the flow-through cell dissolution test, the drug was dissolved from the patch with a higher drug content at a much higher rate, and the dissolution rate returned to a uniform 2 mg/cm² after 3 hours. The initial high drug release is due to the regulator layer being initially saturated with capsaicin from the underlying drug-containing layer (i.e., the layer farther from the skin surface). In the first hour of the dissolution test, the drug first dissolves from this saturated layer. The rate of dissolution is high because of the short diffusion path of the drug. The drug released from the regulator layer is replaced by the active ingredient dissolved in the layer below, but due to the increase of the diffusion distance, the drug release value decreases, and then remains constant when equilibrium is reached. The release value is the rate of release, preferably a rate measured by any method described herein.

In the case of a patch with a lower drug content, the regulator layer cannot be saturated with capsaicin. Drug release starts from a lower level and this value decreases slightly but continuously over time because of the increasing diffusion pathway.

In consequence, the phenomena observed in the dissolution test are due to the fact that the regulator layer of the patch with a higher concentration is saturated or essentially saturated with the drug (preferably at least 80% of the saturation concentration, more preferably at least 90% of the concentration, which is made possible by the fact that the released active ingredient is replaced by the dissolution of the active ingredient present essentially as a pure substance, preferably as a solid substance) and continuously replenishes the capsaicin flowing into the liquid medium due to diffusion from the drug-containing layer. This replacement does not occur in a patch with a lower drug content, and the increasing diffusion distance cannot be compensated for by the diffusion rate. On the other hand, it can be stated that the regulator layer fulfills its expected function in both compositions. Instead of the exponential concentration decrease observed with matrix diffusion patches, a steady, only slowly decreasing release curve with essentially zero-order kinetics can be observed. A similar trend was shown in the drug release studies (IVRT) in the Franz cell, with significantly lower drug release from the patch containing capsaicin with a lower dose. According to the human dermal permeation study (IPVT), the permeation of the drug was more superficial from the patch containing the lower dose of the drug, and the penetration of capsaicin also required a longer time. In patches with a higher active ingredient content, the compound already passes through the skin in half of the administration time. The amount of the transferred active ingredient is much higher than proportionally expected. (See e.g. FIG. 11.) The results show that in the case of the layer combination we used, the patch with a higher drug content more closely approximated the desired zero-order drug release kinetics. Prolonged, controlled release was also confirmed by Raman microscopic examinations. The drug penetrated evenly and permanently into the skin tissue from the patches with higher capsaicin content.

The devices and methods used to make the patches model industrial production. Therefore, the applied technologies can be adapted to small-scale production with minor modifications.

Patches containing large concentration (8%) of capsaicin are approved for the treatment of neuropathic pain, such as postherpetic neuralgia and diabetic neuropathy (1. Press Release: Grünenthal and Averitas Pharma Announce U.S. FDA Approval of QUTENZA for the Treatment of Neuropathic Pain Associated with Diabetic Peripheral Neuropathy of the Feet. Jul. 21, 2020. EMA. Qutenza. Available at: www.ema.europa.eu/en/medicines/human/EPAR/qutenza). The mechanism of action of these patches involves a strong and sustained activation of transient receptor potential vanilloid 1 (TRPV1) ion channels in primary nociceptor nerve endings. Opening of the channels causes a pathologically increased intracellular Ca²⁺ concentration in nerve endings, leading to cytoskeletal and mitochondrial damage. Nociceptor nerve endings are defunctionalized for 12-14 weeks, providing long-lasting analgesia [Bley, K. R. et al. 2010; Kaale, E, et al., 2002]. Thus, the nociceptor function of the nerve fibers and SP release, believed to be an important signal for pain neurotransmission, also become impaired for an extended period of time [Bley, K. R. et al. 2010; Kaale, E, et al., 2002]. These processes have been considered as potential mechanisms of analgesic action of topical capsaicin treatment, but several clinical studies proved that SP receptor antagonists had no pain-relieving effect [Hill, 2000]. In their 2011 article, Anand and Bley suggest that capsaicin has only a limited potential for transdermal delivery across human skin, thus it causes only defunctionalization of the cutaneous nociceptors [Anand and Bley (2011)]. Desensitization by topical capsaicin affects only cutaneous nociceptors, limiting the use of transdermal systems comprising a high dose of capsaicin.

Our data corroborate that transdermal systems containing much lower capsaicin concentrations than the dose that defunctionalizes the receptor can also be effective against neuropathic pain. A patch containing only 0.04% capsaicin alleviated postherpetic neuropathic pain in 60.1% of the patients, 28.2% of whom exhibited increased analgesia throughout 12 weeks [Martini, C. H. 2013]. A mixed patient group suffering from either postherpetic or diabetic neuropathic pain experienced analgesic effect of a transdermal patch containing 0.625% capsaicin [Moon, J.-Y. 2017].

Following our unexpected and non-obvious finding according to the invention, we searched for a complex answer to how such a small capsaicin content can exert effective analgesia. The antinociceptive effect developed in the deeper musculoskeletal and joint areas could not be explained by the desensitization of the cutaneous afferents [Anand and Bley (2011)]. Activation of TRPV1 ion channels and consequent increase in intracellular Ca²⁺ concentration induces neuropeptide release, but does not damage the nerve endings. Many of these peptides contribute to vasodilatation and plasma extravasation (e.g., substance P and calcitonin gene-related peptide). Other peptides, like somatostatin or endogenous opioids, might possess analgesic and anti-inflammatory actions [Pethö, G. et al. 2017]. The systemic antinociceptive effect of somatostatin has been proved in animal studies [Szolcsányi, J et al., 2004; Spampinato, S. et al. 1988]. According to human data, the systemic analgesic effect of topical capsaicinoid treatment is related to a remarkable increase of somatostatin concentration in the plasma [Horváth, K. et al., 2014].

Besides the peripheral action, somatostatin exerts a central analgesic effect, as well [Spampinato, S. et al. 1988]. Somatostatin-immunoreactive structures were detected in lamina II of the lumbar spinal cord of the rat, which were proposed as the anatomical basis of somatostatin-induced analgesia [Rosenthal, B. M. et al. 1989]. Expression of SSTR4 receptor mRNA was detected at various levels of the murine and human neuronal pathways of pain sensation [Kecskés, A. et al. 2020]. The analgesic effect of somatostatin, including neuropathic pain, could be reproduced by selective SST4 receptor agonist [Kántás, B et al., 2019, Sándor, K. et al. 2006]. Thus, it is plausible that the capsaicin used in a low-dose, prolonged-release patch according to the invention exerts its analgesic effect through the release of somatostatin.

In summary, the transdermal patch described in the present work offers the possibility of low-dose topical capsaicin treatment without contaminating the hands or clothing, and allows accurate administration by cutting the patches to size.

Additionally, our technology offers superior release kinetics that can be exploited with other drugs. Our transdermal system was subjected to routine—Franz-cell, Raman-spectroscopy—and self-developed—flow-through cell—in vitro tests. In our opinion, the conditions of flow-through cells more accurately model the processes taking place in the skin tissues, and thus the release of the active ingredient from the patch. We chose well-established animal models of nociception to study our transdermal system. The increasing temperature water bath is not a widely known method, despite its validation in the surgical plantar incisional pain model with opioids and NSAIDs [Füredi, R. et al., 2019]. Exposing the entire sole surface to heat is a major advantage in the surgical incision model, as different areas of the sole may exhibit different degrees of hyperalgesia. This is a serious challenge in practice, if not impossible for mechanical testing. On the other hand, classic carrageenan-induced plantar inflammation allows easy and effective testing of mechanical hyperalgesia with dynamic plantar aesthesiometry [Bátai, I. Z. et al., 2018]. Animals subjected to carrageenan-induced plantar inflammation do not show thermal hyperalgesia. These methods may also be suitable for testing other transdermal therapeutic systems.

Our silicone-based TTS displayed long-lasting, controlled, dose-dependent release and permeation of capsaicin. TTS containing a higher dose (2.3 mg/g) of capsaicin is capable to deliver the active ingredient to the target receptors in the dermis and exert a systemic antinociceptive effect. We presume that activation of TRPV1 ion channels on the sensory nerve endings in the patch-treated dorsal skin triggers the release of inflammatory neuropeptides such as SP and CGRP, inducing a local temperature increase and painful redness. In addition, antinociceptive mediators, such as somatostatin and opioid peptides, are also released from the central peripheral endings of primary afferents, regulating the pain pathway. The systemic analgesic effect of the patch comprising low-dose capsaicin can be explained by these sensocrine regulatory mechanisms. Further experiments involving TTS comprising various detergents and other excipients possessing unexplored potential offer further optimization of substance release and increased therapeutic value.

### EXAMPLES

Our invention is described in more detail below by way of examples—without limiting the scope of the invention exclusively to the raw materials and/or compositions and/or conditions specified therein. The % values given in the examples refer to % by weight everywhere, unless it is defined differently.

### Example 1—Capsaicin Patch with 2 Layers

A powder dilution (trituration) is prepared from 0.1005 g of capsaicin with 4.9959 g of CaCO₃, and 0.2360 g of capsaicin is dissolved by heating in 10.3164 g of 85% glycerol (stock solution) and these are used to prepare the patch.

1.5075 g of 85% glycerol, 20 g of polysorbate 20 and 9.1260 g of Elastosil RT-601 component A (Wacker Chemie GmbH) are mixed in a glass beaker, then 0.4849 g of capsaicin trituration (9.56 mg capsaicin) and 0.4946 g of capsaicin stock solution (11.31 mg of capsaicin) are added to the mixture, then mixed until complete homogeneity. Then 1.0304 g of Elastosil RT-601 component B is added and the mixture is homogenized again. This mixture is then spread on a 0.04 mm thick paper substrate laminated on aluminum in a layer thickness of 0.4 mm and the silicone components are crosslinked at a temperature of 70° C. for 30 minutes (first layer).

After that, 0.7244 g of 85% glycerol, 0.2421 g of polysorbate 20, 4.4583 g of Elastosil RT-601 component A and 0.5824 g of Elastosil RT-601 component B is measured into a glass beaker, then the components are homogenized by mixing. Then the mixture is layered on the first layer in a layer thickness of 0.2 mm and the silicone components are crosslinked at 70° C. for 30 minutes (second layer).

The next day, Liveo BIO-PSA 7-4301 pressure-sensitive adhesive manufactured by DUPONT is layered onto the surface of the second layer in a layer thickness of 0.2 mm, and then left in a dust-free and well-ventilated space to allow the solvent (hexane) content of the adhesive to evaporate.

### Example 2—Capsaicin Patch with 1 Layer

1.5094 g of 85% glycerol, 0.8169 g of polysorbate 20 and 9.0110 g of Elastosil RT-601 component A (Wacker Chemie GmbH) are mixed in a glass beaker, then 1.4911 g of capsaicin trituration (29.4 mg of capsaicin) and 0.8004 g of capsaicin stock solution (18.2 mg of capsaicin) are added to the mixture, then mixed until complete homogeneity. Then 1.0018 g of Elastosil RT-601 component B is added and the mixture is homogenized again. This mixture is then spread on a 0.06 mm thick, soft PVC support in a layer thickness of 0.5 mm and the silicone components are crosslinked at a temperature of 50° C. for 60 minutes. Then, Liveo BIO-PSA 7-4202 pressure-sensitive adhesive manufactured by DUPONT is layered onto the surface of the layer in a layer thickness of 0.3 mm, and then left in a dust-free and well-ventilated space to allow the hexane content of the adhesive to evaporate.

### Example 3—Capsaicin Patch with 1 Layer

0.0521 g of capsaicin is dissolved in 1.0 ml of 96% alcohol, then 2.3272 g of 85% glycerol and 0.1248 g of CAB-O-SIL hydrophilic colloidal silica manufactured by Cabot are added, then the components are homogenized with intensive mixing. Then, 9.0907 g of polydimethylsiloxane-α,ω-diol with a viscosity of 5,000 mPas is added to the mixture and the mixture is homogenized by stirring again. Finally, 1.0102 g of Oxam crosslinking catalyst (T-Szilox Kft., Hungary) is added to the mixture and it is mixed into the mixture, as well. The homogeneous mixture thus obtained is spread in a layer thickness of 0.6 mm on the surface of a 0.04 mm thick paper laminated with aluminum and crosslinked for 60 minutes at room temperature. The next day, Liveo BIO-PSA 7-4101 pressure-sensitive adhesive manufactured by DUPONT is layered onto the surface of the layer in a layer thickness of 0.3 mm, and then left in a dust-free and well-ventilated space to allow the hexane content of the adhesive to evaporate.

### Example 4—Capsaicin Patch with 2 Layers

The layer according to Example 3 is prepared with a layer thickness of 0.4 mm, and after 1 hour of crosslinking, a mixture consisting of the following components is layered onto its surface in a layer thickness of 0.1 mm: 0.7173 g of 85% glycerol, 4.5684 g of polydimethylsiloxane-α,ω-diol with a viscosity of 20,000 mPas and 0.5089 g of Oxam crosslinking catalyst (T-Szilox Kft., Hungary). The layer is left to crosslink at room temperature. The next day, Liveo BIO-PSA 7-4302 pressure-sensitive adhesive manufactured by DUPONT is layered onto the surface of the layer in a layer thickness of 0.4 mm, and then left in a dust-free and well-ventilated space to allow the hexane content of the adhesive to evaporate.

### Example 5—Capsaicin Patch with 1 Layer

0.0203 g of capsaicin is dissolved in 1.0 ml of 96% alcohol, then 2.0388 g of 85% glycerol and 0.1203 g of CAB-O-SIL hydrophilic colloidal silica manufactured by Cabot are added, then the components are homogenized with intense mixing. Then, 9.0680 g of polydimethylsiloxane-α,ω-diol with a viscosity of 5,000 mPas is added to the mixture and the mixture is homogenized by stirring again. Finally, 1.0050 g of prehydrolyzed tetraethoxysilane crosslinking component under the brand name TES-40 (Wacker Chemie GmbH, Germany) is added to the mixture and it is mixed into the mixture, as well. The homogeneous mixture thus obtained is spread in a layer thickness of 0.4 mm on the surface of a 0.04 mm thick paper laminated with aluminum and crosslinked for 30 minutes at room temperature in an atmosphere containing diethylamine. After that, the adsorbed amine is removed from the layer by placing it in a space with intense air exchange. The next day, Liveo BIO-PSA 7-4101 pressure-sensitive adhesive manufactured by DUPONT is layered onto the surface of the layer in a layer thickness of 0.4 mm, and then left in a dust-free and well-ventilated space to allow the hexane content of the adhesive to evaporate.

### Example 6—Capsaicin Patch with 2 Layers

The layer according to Example 5 is prepared, and after crosslinking and venting, a mixture consisting of the following components is layered onto its surface with a layer thickness of 0.3 mm: 0.7187 g of 85% glycerol, 4.5274 g of polydimethylsiloxane-α,ω-diol with a viscosity of 20,000 mPas and 0.5192 g of TES-40 prehydrolyzed tetraethoxysilane crosslinking component (Wacker Chemie GmbH, Germany). The layer is crosslinked for 30 minutes at room temperature in an atmosphere containing diethylamine. After that, the adsorbed amine is removed from the layers by placing them in a space with intense air exchange. The next day, Liveo BIO-PSA 7-4102 pressure-sensitive adhesive manufactured by DUPONT is layered onto the surface of the layer in a layer thickness of 0.5 mm, and then left in a dust-free and well-ventilated space to allow the hexane content of the adhesive to evaporate.

Data of the patches according to Examples 1 to 6 are summarized in Table 1.

TABLE 1

| | | | | | | Function of | Possible |
|---|---|---|---|---|---|---|---|
| | | | | | | the given | alterna- |
| Example 1 | Example 2 | Example 3 | Example 4 | Example 5 | Example 6 | component | tives |
| capsaicin, CaCO₃, | | capsaicin dissolved in alcohol | | | | active | |
| 85% glycerol | | | | | | ingredient | |
| 85% glycerol | | 85% glycerol | | s | | liquid | abs. |
| | | | | | | excipient | glycerol |
| | | | | | | (polar) | (100%) |
| polysorbate 20 | | | | | | emulsifier, | |
| | | | | | | tenside | |
| Elastosil RT-601 | | polydimethylsiloxane-α,ω-diol | | polydimethylsiloxane-α,ω-diol | | raw material of | |
| component A | | with a viscosity of 5000 mPas | | with a viscosity of 5000 mPas | | silicone layer | |
| Elastosil RT-601 | | | | | | raw material of | |
| component B | | | | | | silicone layer | |
| capsaicin trituration | | | | | | introducing, | |
| and stock solution | | | | | | diluting active | |
| | | | | | | ingredient | |
| | | CAB-O-SIL hydrophilic | | CAB-O-SIL hydrophilic | | solid excipient | |
| | | colloidal silica | | colloidal silica | | | |
| | | Oxam crosslinking catalyst | | | | mixture of | |
| | | | | | | crosslinking | |
| | | | | | | agent + catalyst | |
| | | | | TES-40 prehydrolyzed | | crosslinker | |
| | | | | tetraethoxysilane crosslinker | | | |
| 85% glycerol | | | 85% glycerol | | 85% glycerol | liquid excipient | abs. |
| | | | | | | (polar) | glycerol |
| | | | | | | | (100%) |
| polysorbate | | | | | | emulsifier, | |
| 20 | | | | | | tenside | |
| Elastosil | | | polydimethyl- | | polydimethyl- | raw material of | |
| RT-601 A | | | siloxane-α,ω-diol | | siloxane-α,ω-diol | silicone layer | |
| Elastosil | | | with a viscosity | | with a viscosity | raw material of | |
| RT-601 B | | | of 20000 mPas | | of 20000 mPas | silicone layer | |
| | | | Oxam | | | mixture of | |
| | | | crosslinking | | | crosslinking | |
| | | | catalyst | | | agent + catalyst | |
| | | | | | TES-40 | crosslinker + | |
| | | | | | prehydrolyzed | catalyst | |
| | | | | | tetraethoxy-silane + | | |
| | | | | | diethylamine | | |
| DUPONT | DUPONT | DUPONT | DUPONT | DUPONT | DUPONT | pressure- | Wacker |
| Liveo | Liveo | Liveo | Liveo | Liveo | Liveo | sensitive | RT-612 |
| BIO-PSA | BIO-PSA | BIO-PSA | BIO-PSA | BIO-PSA | BIO-PSA | adhesive | A + B |
| 7-4301 | 7-4202 | 7-4101 | 7-4302 | 7-4101 | 7-4102 | | |

### Example 7—the Active Ingredient Release Testing of Patches According to Examples 1 to 6

The active ingredient release of the patches according to Examples 1 to 6 was examined under laboratory conditions. The samples were placed in a cell in which a surface area of 12.56 cm² of the samples was in contact with a continuously changing dissolving liquid (release liquid). The amount of solution in contact with the surface per hour was 20 cm², distilled water containing 5% glycerol was used as the dissolving liquid. The capsaicin content of the dissolving liquid was determined by UV spectroscopy, at a wavelength of 227 nm, in a 1 cm thick quartz cuvette, using a calibration curve. The active ingredient release curves of the patches according to Examples 1 to 6 are shown in FIGS. 15 to 20, respectively.

Based on the results of the test, the advantage of the transdermal patch according to the invention is that, when placed on the patient's skin, it can ensure the delivery of the active ingredient into the skin at a uniform rate for 6 to 12 hours, in the form of patches of different sizes. In the case of a preparation with a given composition, the desired dose can be achieved by using a patch of the appropriate size, the patch can be cut to size. A significant advantage of using low-dose capsaicin patches with zero-order kinetics compared to high-dose patches is that they are able to deliver capsaicin into the deeper layers of the skin over a long period of time, and as a result, a pain-relieving effect can be observed. Their significant advantage is that their effect lasts only as long as the patch is on the skin, while high-dose patches desensitize the given skin area for a long time, and the loss of pain sensation can have serious consequences.

### Examples 8 to 10—In Vitro Release and Transdermal Penetration of Capsaicin—Materials and Methods

In the experiments described in Examples 8 to 10 involving patches with capsaicin concentrations of 1 mg/g and 2.3 mg/g, the in vitro release and transdermal penetration of capsaicin were measured with Franz diffusion cells and Raman microscopy (Example 9). The antinociceptive effect of TTS was also tested in vivo in animal studies (Example 10). Thermal hyperalgesia was measured in response to surgical incision of the hind paw, and carrageenan-induced plantar inflammation, which induced mechanical allodynia, was detected by dynamic plantar aesthesiometry in rats.

### Example 8—Experiments Involving Patches with Capsaicin Concentrations of 1 mg/g and 2.3 mg/g—Materials and Methods

### Chemicals

RT-601 A™—addition crosslinkable polydimethylsiloxane-(α,ω)-divinyl and RT-601 B™ crosslinker were from Wacker GmbH, Germany. Glycerol was purchased from Molar Chemicals Ltd., Hungary. Polysorbate 20 was from Hungary. Capsaicin was from Chillies Export House Limited, India.

### Production of Capsaicin-Containing Transdermal Patches

TTS samples used in animal experiments were prepared on a paper substrate laminated on aluminum foil of 0.4 mm thickness. Capsaicin was mixed into silicone matrix carriers. Our raw polymer was RT-601 A™—addition crosslinkable polydimethylsiloxane-(α,ω)-divinyl. Capsaicin was dissolved in glycerol by heating and was added to the silicone raw material. Crystalline capsaicin was also added to our mixture. Capsaicin was diluted with calcium carbonate. Calcium carbonate as an inert excipient was added to the samples only to ensure accurate balancing. If needed, liquid glycerol and polysorbate 20 were added, too. RT-601 B crosslinker was added to the mixture under stirring. After the components were weighed, mixtures were homogenized and spread on the supporting film in a thickness of 0.4 mm. The layer was crosslinked at a temperature of 70° C. The procedure was finished in 60 minutes. After that, we spread a second, regulator layer (FIG. 1), which did not contain capsaicin, only glycerol and polysorbate, in addition to the siloxane raw polymer. The second layer was crosslinked at a temperature of 70° C. for 60 minutes. Samples were rested for 48 hours and examined afterwards. Two preparations were prepared, a sample containing a lower (1 mg/g capsaicin) and a higher (2.3 mg/g capsaicin) dose of capsaicin.

The composition of the 1 mg/g and 2.3 mg/g samples was as follows (see Table 2).

TABLE 2

| | |
|---|---|
| Low-dose capsaicin patch | High-dose capsaicin patch |

1^{st} layer with 0.4 mm thickness

| | | |
|---|---|---|
| Capsaicin | 3.60% | 8.36% |
| trituration | | |
| Capsaicin | 3.75% | 5.58% |
| solution | | |
| Glycerol | 13.16% | 10.52% |
| Polysorbate 20 | 3.95% | 5.69% |
| RT 601 A | 69.32% | 62.84% |
| RT 601 B | 7.83% | 6.98% |

2^{nd} layer (regulator) with 0.1 mm thickness

| | | |
|---|---|---|
| Glycerol | 12.05% | 11.88% |
| Polysorbate 20 | 4.0% | 3.43% |
| RT 601 A | 74.21% | 76.21% |
| RT 601 B | 9.69% | 8.46% |
| Total capsaicin | 1 mg / g patch | 2.3 mg / g patch |

### Measurement of the In Vitro Release of Capsaicin-Containing Transdermal Patches

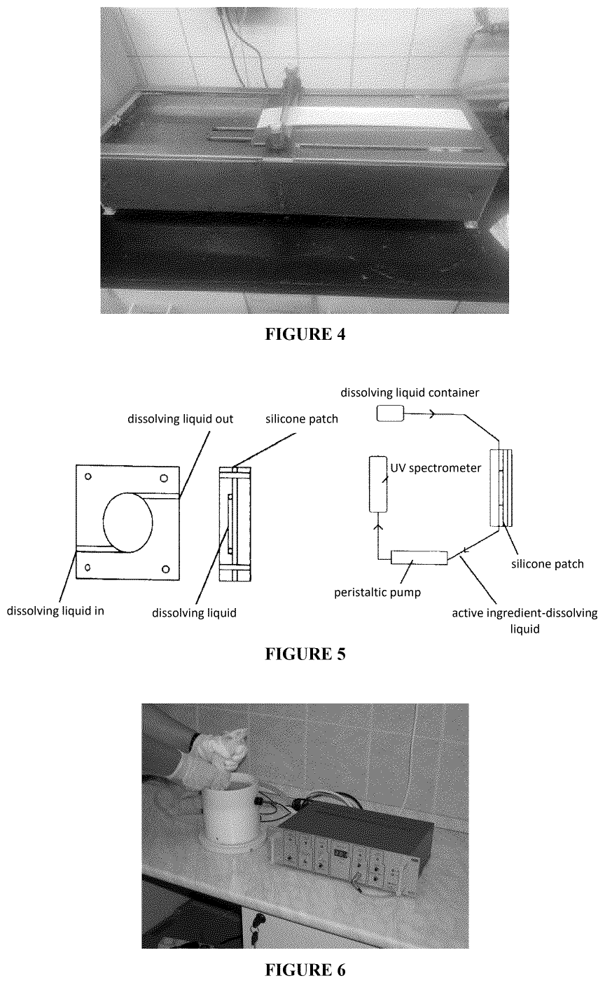

In vitro testing was performed in two ways. First, we measured in a Franz cell [Ng, S-F. et al. 2010], which models static and vertical subcutaneous drug dissolution. In the second method, patches were examined in a flow-through cellular device that mimics the dissoluted drug concentration in the blood (FIG. 5).

Investigation of Drug Release and Permeation with a Franz Diffusion Cell

In vitro release tests (IVRTs) and in vitro permeation tests (IVPTs) were performed. A vertical Franz-type diffusion cell (Hanson Microette T M Topical & Transdermal Diffusion Cell System, Hanson Research Corporation, USA) was used to model the capsaicin release from the patches in case of IVRT and drug permeation through human heat-separated epidermis (HSE) in case of IVPT. The HSE was prepared as follows: excised human subcutaneous fat-free skin was placed in a water bath (60±0.5° C.) and the epidermis was separated from the dermis. About 250 mg of patch (1.77 cm²) was used as the donor phase. The patches were placed in the donor chamber directly in the case of IVRT. In the case of IVPT, the donor and receptor phases were separated by HSE.

The receptor phase was thermostated phosphate buffer (PBS, pH 7.4±0.15) and 25% w/w ethanol (96 V/V %) at 32° C.±0.5° C. The investigation lasted for 24 hours. The stirring speed was 450 rpm. The concentration of the drug was examined by high-performance liquid chromatography (HPLC). HPLC analysis was carried out on a Shimadzu NEXERA X2 HPLC system (Shimadzu Corporation, Tokyo, Japan). Kinetex C18 150 mm×4.6 mm packed with 3 μm (Phenomenex Inc., Torrance, CA, USA) column was used. Acetonitrile in a ratio of 30:70 with a flow rate of 1 mL/min was applied during the isocratic elution with HPLC-grade water. Prior to the elution, the eluent was degassed and filtered through a glass filter funnel with a pore size of 0.45 μm. The run time was 4 minutes, the retention time of capsaicin was 2.3 minutes. Detection was performed via absorption at 280±4 nm. A sample volume of 20 μL was injected, and the elution was carried out at a sample temperature of 25° C. and at a column temperature of 45° C.

### Mathematical Evaluation

The data are the averages±standard deviation of the results of 6 experiments. The release and permeation profiles of the patches were obtained. The cumulative amount of capsaicin released and permeated per cm² at 24 hours was calculated. The flux (J) was the slope of the cumulative amounts of released and permeated capsaicin (μg/cm²) versus square root of time (h^{1/2}) in the case of the IVRT, and it was the slope versus time (h) in the case of the IVPT [Zsiko S et al. 2019].

### Flow-Through Cell

Samples (12.56 cm² each) of patches were tested for modified IVRT in a flow-through cell (4 cm diameter and 5 cm³ sample volume) at 37° C. The flow rate (PBS, 5% w/w glycerol) was 25 mL/h, and the capsaicin content was determined hourly with a spectrophotometer (Perkin-Elmer Lambda 25). Detection was performed at a wavelength of 227 nm, in a quartz cuvette with a layer thickness of 1 cm.

Investigation of Skin Permeation with Raman Microscopy

The excised human subcutaneous fat-free skin (epidermis and dermis) was obtained from a Caucasian female patient who underwent abdominal plastic surgery. Samples of patches (1.77 cm²) were placed on the skin surface for 3 hours at 32° C. The treated skin samples were frozen (10 μm thick cross-section) with a Leica CM1950 cryostat (Leica Biosystems GmbH, Wetzlar, Germany).

The microtomed skin samples were placed on an aluminum surface, with the subcutaneous part towards the top of the plate.

Raman spectroscopic measurements were made with a Thermo Fisher DXR Dispersive Raman Spectrometer (ThermoFisher Scientific Inc., Waltham, MA, USA) equipped with a CCD camera and a diode laser.

During the measurements, a laser light source of 780 nm wavelength was used, with a maximum power of 24 mW, minimizing the effect of fluorescence. The microscopic lens used for the measurements had a magnification of 50 times, and the aperture of the pinhole was 25 μm. In the case of chemical mapping, an area of 200×1800 μm was examined; the step size was 50 μm vertically and horizontally. Each spectrum was produced from 16 scans with an exposure time of 2 seconds. Altogether 205 spectra were registered. Analyzing the treated and untreated skin samples, capsaicin was used as a reference. Data acquisition and analysis were accomplished using OMNICTM8.2 for Dispersive Raman software package (ThermoFisher Scientific Inc., Waltham, MA, USA).

### Animals

Male Wistar rats of 125-150 g weight were purchased from ToxiCoop Zrt., Hungary. The rats were kept at the Department of Pharmacology and Pharmacotherapy, Medical School, University of Pécs under standard pathogen-free conditions with freely available food pellets and water. Experiments conform to the 40/2013, II. 14. Hungarian Government Regulation on the Protection of Animals Used for Scientific Purposes, to the Directive 2010/63/EU of the Council of the European Communities and to the requirements of the International Association for the Study of Pain (IASP). Experiments were approved by the Ethics Committee on Animal Research of the University of Pécs (license number BA02/2000-8/2018, 18.8.2018). The dorsal skin of the animals was epilated with a commercial epilation cream from the nape to the hips under ketamine and xylazine anesthesia (80 and 10 mg/kg i.p.) 2 days before the animals participated in the experiments.

### Surgical Incision of the Hind Paw

The animals were anesthetized with sodium pentobarbital (50 mg/kg i.p.). The plantar surface of one hind paw was treated with povidone-iodine. Sides subjected to surgery were randomized. The paw was incised at a wound length of 10 mm with a scalpel. The depth of the incision reached the muscle layer.

The wound was closed with two 6.0 sutures and treated with povidone-iodine [Pethö, G. et al., 2017, Füredi R. et al., 2009]. Capsaicin-releasing transdermal patches were applied according to 2 schedules.

In the first set of experiments, patches were attached to the dorsal skin right after the plantar incision, when the pentobarbital anesthesia still lasted (immediate application).

In other experiments, patches were applied 18 hours after the incision (delayed application). The size of the patches was 3×6 cm. Patches were fixed to the animals with commercial adhesive bandage. Adhesive bandage without patch was used as control. Patches were kept on the animals for 6 hours in both experimental embodiments. After the six-hour interval, thermal pain threshold of the hind paws was measured with increasing temperature water bath (Experimetria Kft., Hungary) [Füredi R. et al., 2009]. Both hind legs of the rats were submerged into the water separately.

The water is heated from 30° C. to 51° C. with a velocity of 24° C./min. The heating is stopped by a foot switch when the animal removes its paw from the water, at which point the device records the temperature of the water. The animals were habituated to handling by the experimenter and to the instrument with three test measurements, and then these were taken as baseline values for the evaluation of the treatment (FIG. 6).

### Carrageenan-Induced Plantar Inflammation

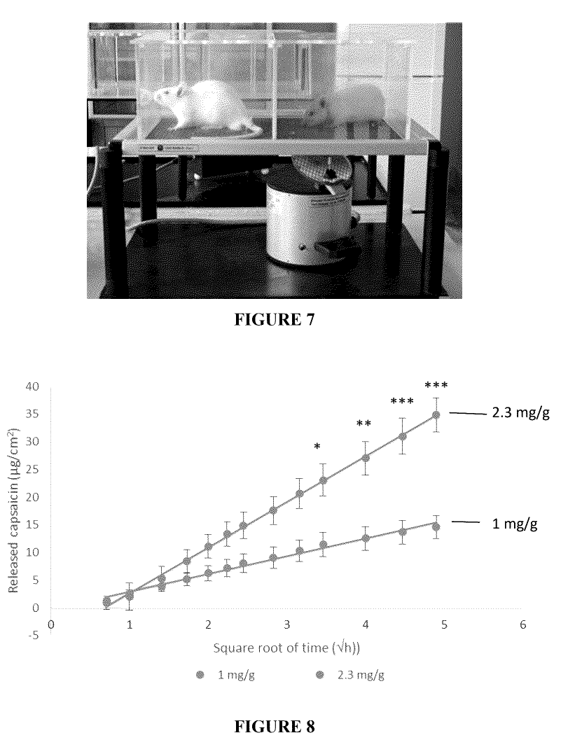

Carrageenan was dissolved in physiological saline under gentle heating (3% w/v). Carrageenan was injected intraplantarly into one hind paw of the rats. Contralateral paw was untreated [Helyes, Z. et al., 2007]. Capsaicin-containing dermal patches were applied to the backs of animals 18 hours after carrageenan injection. The size of patches was 3×6 cm. Patches were fixed to the animals with commercial adhesive tape. Adhesive tape without patch was used as control. Six hours later, mechanical pain threshold of the hind paws was detected by dynamic plantar aesthesiometry (Ugo Basile, Italy). Rats were placed into the compartments of the instrument 10 minutes before the test. The force exerted by the stimulator reached 50 g in 5 seconds. The value inducing nocifensive behavior is automatically displayed. Baseline measurements were performed three times before the actual experiment. Lowered pain threshold was confirmed by detection of mechanical hyperalgesia before the application of transdermal patches (FIG. 7).

### Statistical Analysis

The results were evaluated and analyzed statistically by one-way (in vivo studies) or two-way analysis of variance (in vitro experiments), followed by Bonferroni's multiple comparisons test using Prism for Windows software (GraphPad Software Inc., La Jolla, CA, USA). The data are the mean values of 6 experiments±standard deviation (P<0.05*, P<0.01** and P<0.001*** compared to control) [Helyes, Z. et al., 2007, Bley, K. R. et al., 2010, Kaale, E. et al., 2002].

### Example 9—Experiments Involving Patches with Capsaicin Concentrations of 1 mg/g and 2.3 mg/g—In Vitro Release and Transdermal Penetration of Capsaicin

### Results

Results of Drug Release and Drug Penetration with Franz Diffusion Cells

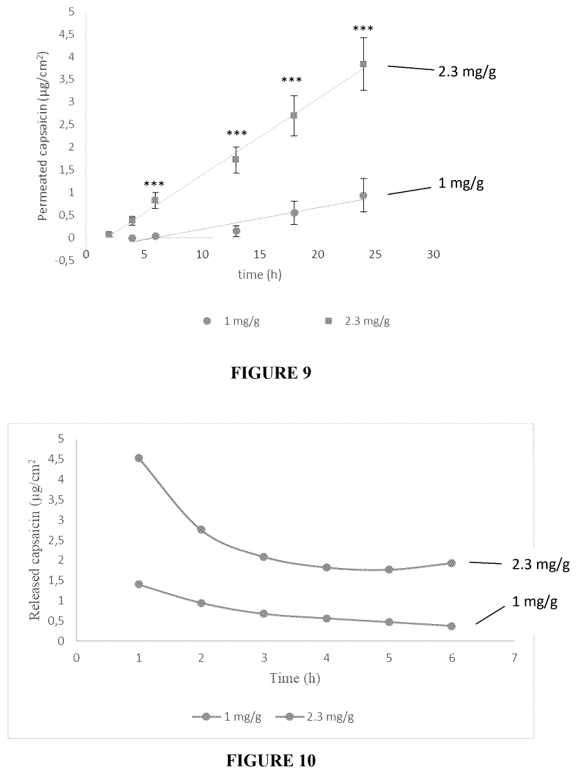

Patches with two different capsaicin contents (1 and 2.3 mg/g) were studied by IVRT (FIG. 8) and IVPT (FIG. 9).

In the IVRT measurement, substantially larger amount of capsaicin was released from the 2.3 mg/g patch within 24 hours than from the 1 mg/g patch. In the IVPT measurement, significantly less drug was delivered to the receptor chamber compared to the IVRT. This is mainly due to the property of the skin that it retains a part of the penetrated active ingredients. The difference between the formulations with two distinct capsaicin contents was similar to that observed in the IVRT experiments. Patches with higher capsaicin concentration yielded higher permeability values. The extent of release (IVRT) itself does not provide relevant information about the penetration of the active ingredient. It is important to examine the permeation through the skin (IVPT) to reveal the interactions of the drug or the drug delivery system with the skin.

The release and permeation profiles were characterized by flux values (Table 3). Flux values show the rate of release and permeation of capsaicin from the different patches.

TABLE 3
Flux values of released and penetrated capsaicin

| | IVRT | IVPT |
|---|---|---|
| 1 mg/g patch | 3.2215 | 0.0466 |
| 2.3 mg/g patch | 8.233 | 0.1672 |

Results of Drug Release with Flow-Through Cell

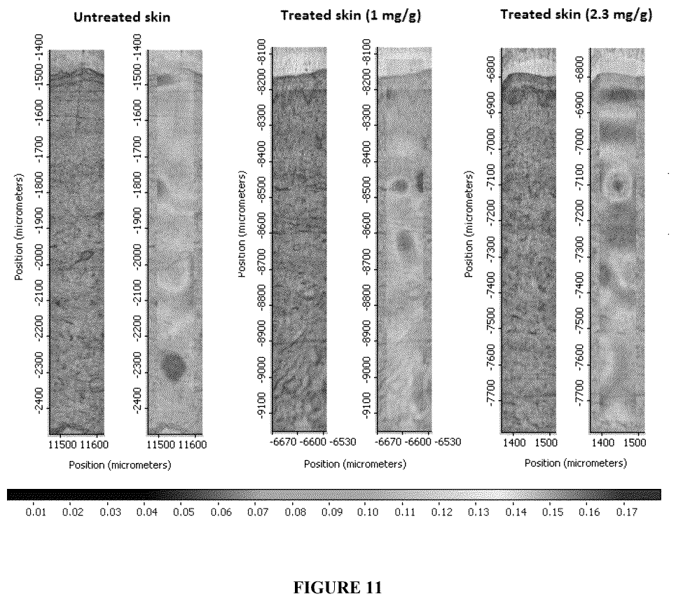

Patches with two different capsaicin contents (1 and 2.3 mg/g) were studied by modified IVRT. In the measurement, substantially larger amount of capsaicin was released from the 2.3 mg/g patches within 6 hours compared to the 1 mg/g patches (FIG. 10). In flow-through cell, a more regulated drug release from the patches was observed. The patch with a higher capsaicin content better regulates the release of the drug over time, i.e. the kinetics of the release of the active ingredient approaches the ideal zero-order kinetic model.

### Results of Raman Spectroscopy

During the Raman experiments, the differences in the localization of capsaicin in different skin regions were determined. The Raman correlation map shows the presence of the penetrated drug in the different layers of human skin, from the skin surface to the dermis, after treatment with patches. Spectral maps were constructed in order to detect the presence of capsaicin in different regions of the human skin. The fingerprint area of the capsaicin spectrum was compared with the treated and untreated human skin spectra.

The Raman correlation maps of the patches are shown in FIG. 11. Raman correlation maps demonstrate the presence of capsaicin in different regions of human skin. In correlation with the IVRT and IVPT results, more effective penetration is seen with the 2.3 mg/g patch than with the 1 mg/g patch. Capsaicin was detected in the dermis and epidermis.

### Example 10—Experiments Involving Patches with Capsaicin Concentrations of 1 mg/g and 2.3 mg/g—In Vivo Animal Studies

### Investigation of the Heat Hyperalgesia Alleviating Effect of Capsaicin-Containing Patches

In experiments using a patch applied immediately after incision of the hind paws, surgical intervention decreased the thermal pain threshold in animals treated with control patches compared to the baseline value, as well as compared to the contralateral intact paw, 6 hours after patch application. Thermal pain threshold of the operated paw in capsaicin-treated rats was still lower than the respective baseline value, but it was significantly larger than the threshold of the operated legs of the control animals with adhesive tape. Thermal sensitivity of the intact paws of capsaicin-treated rats did not differ from the baseline value taken before surgery. The adhesive tape had no effect on the thermal pain threshold of the hind paws (FIG. 12).

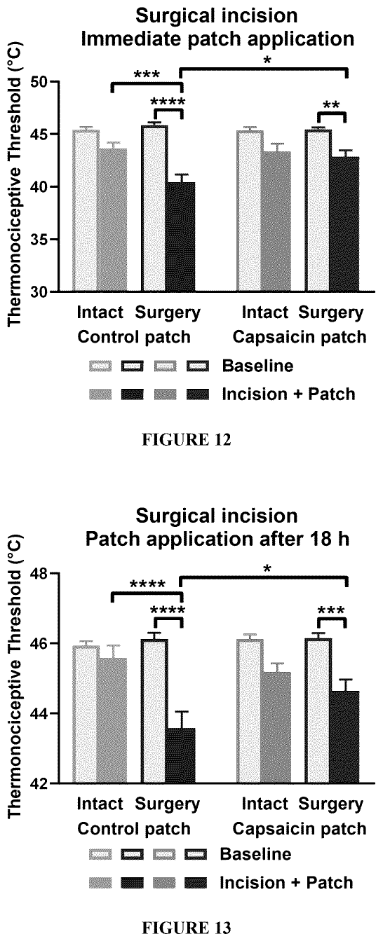

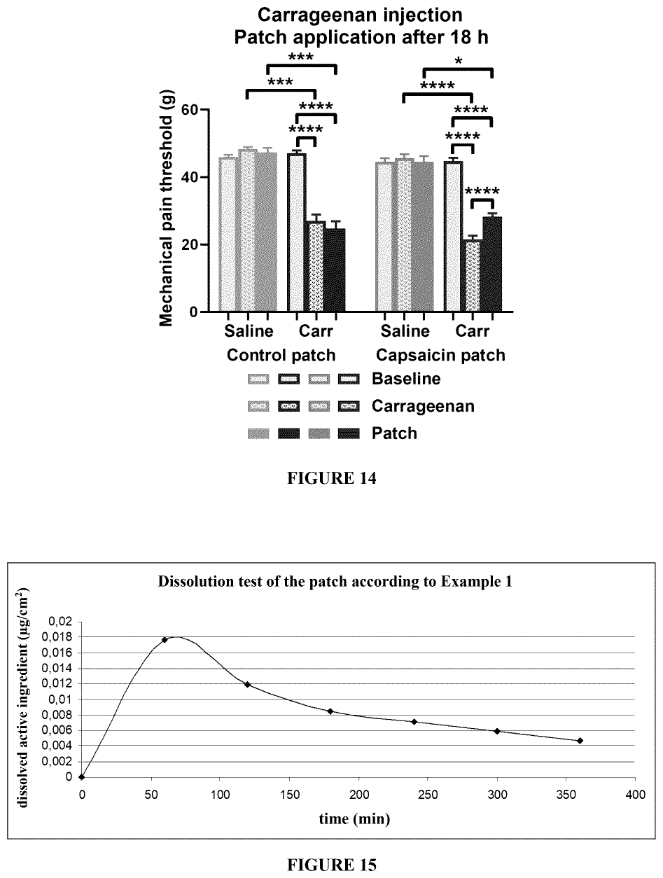

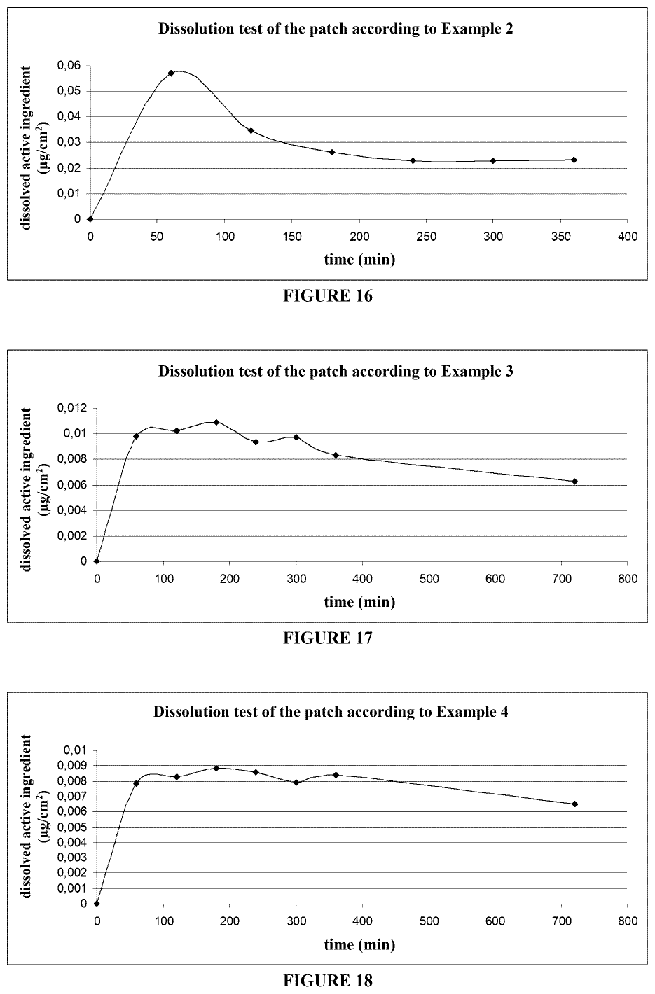

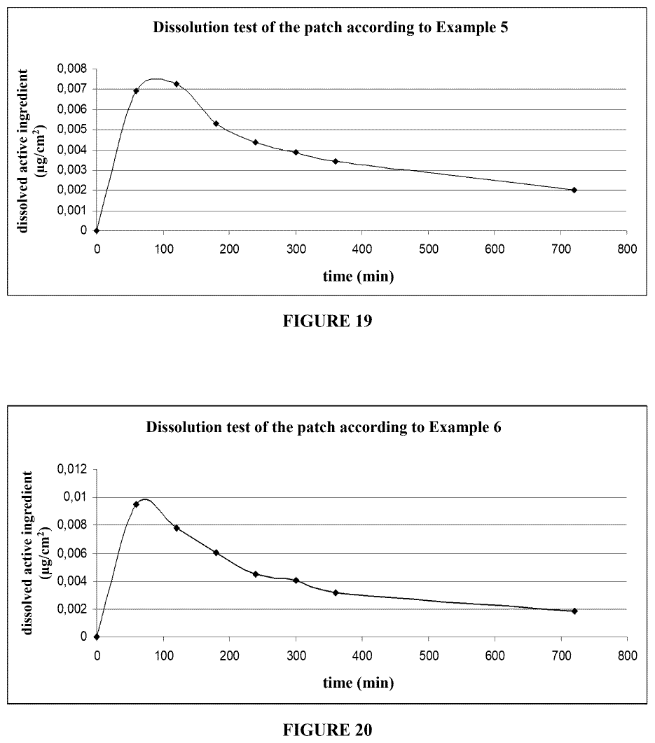

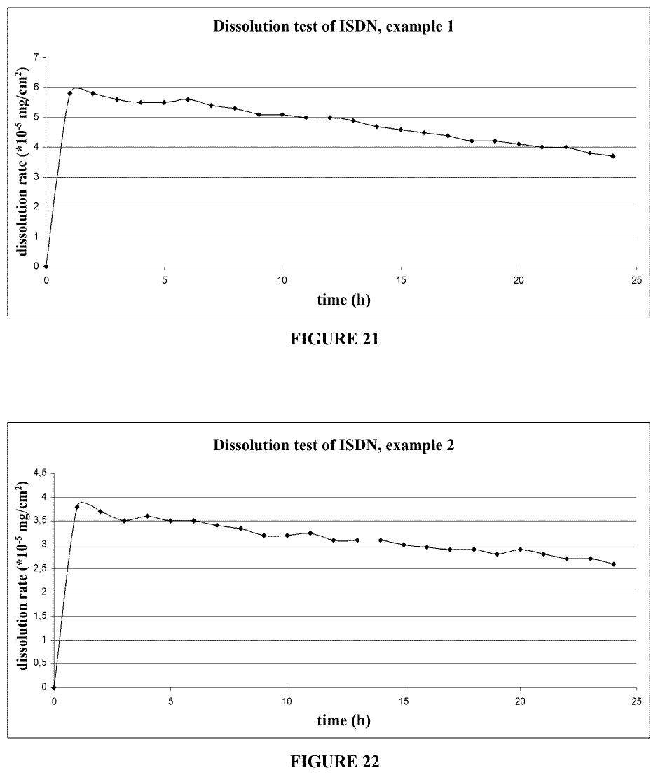

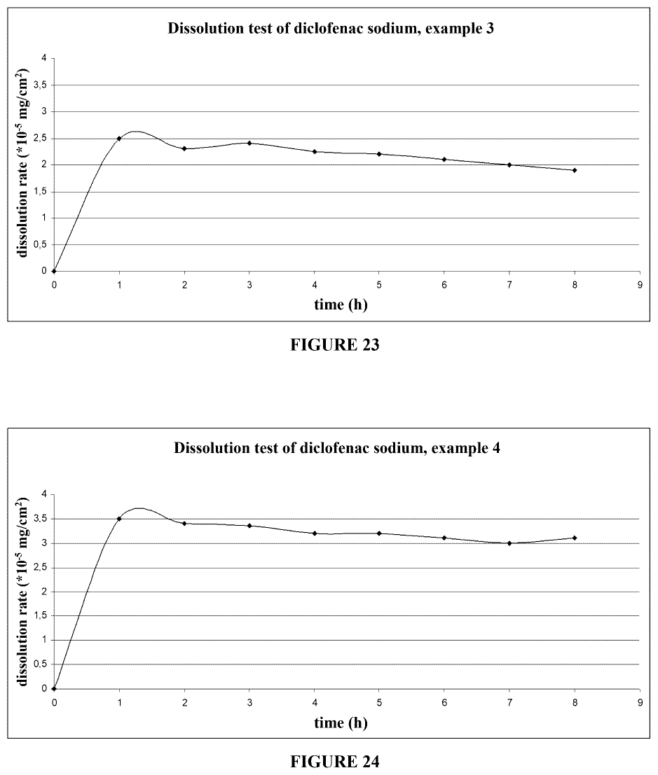

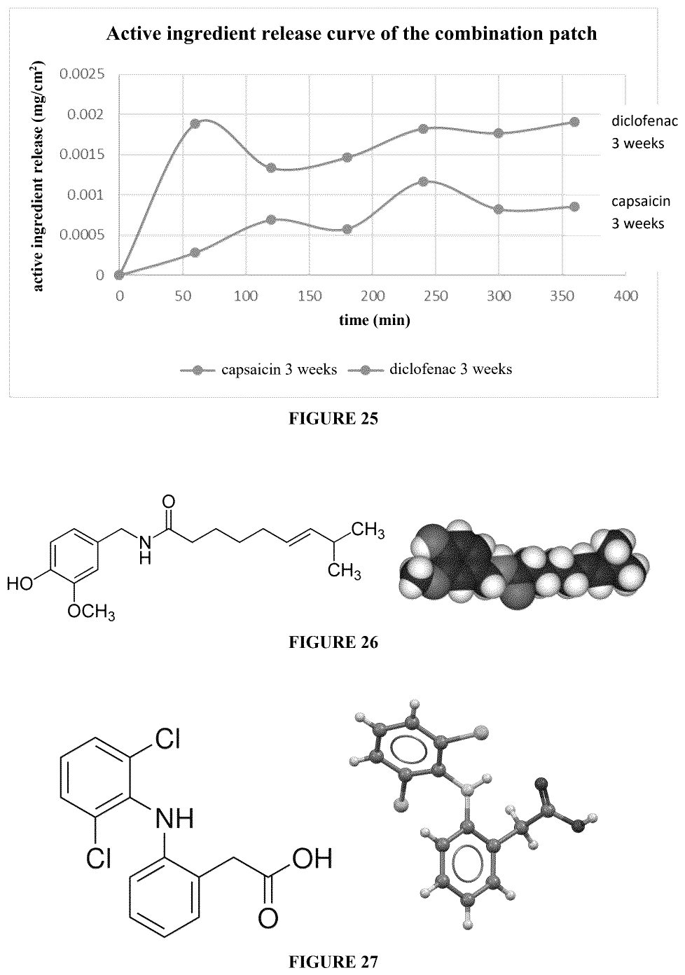

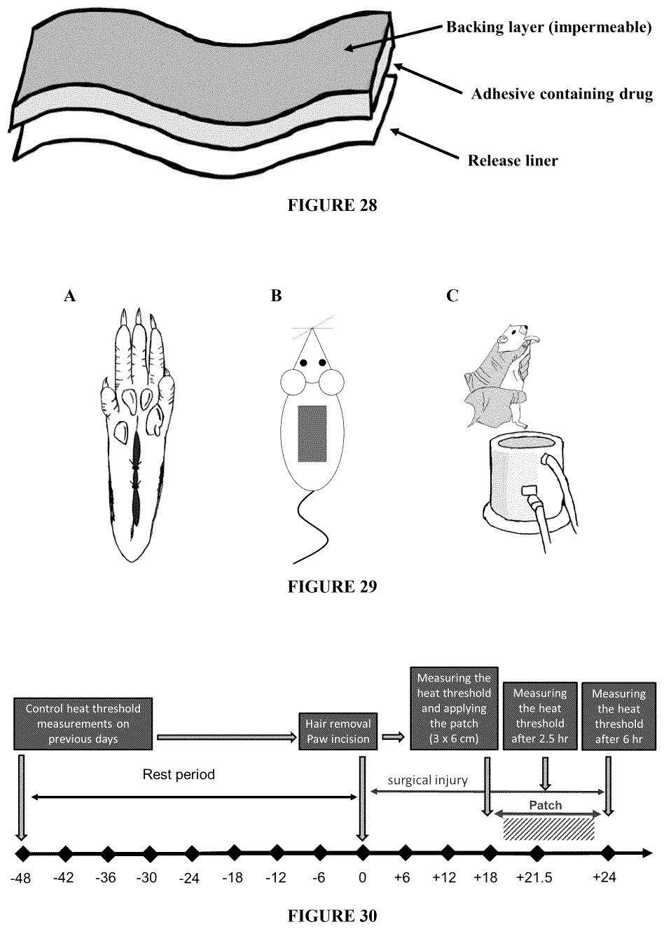

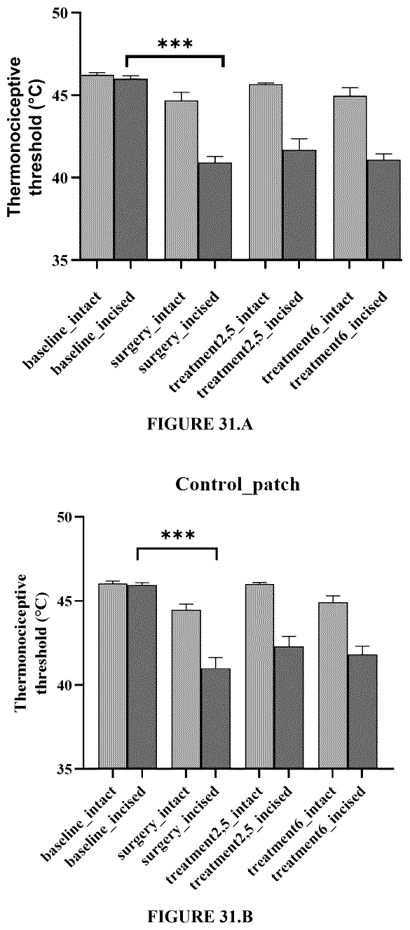

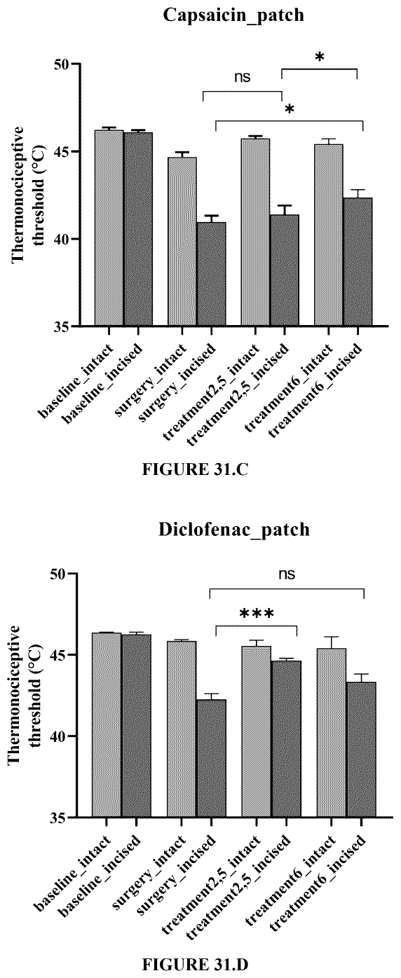

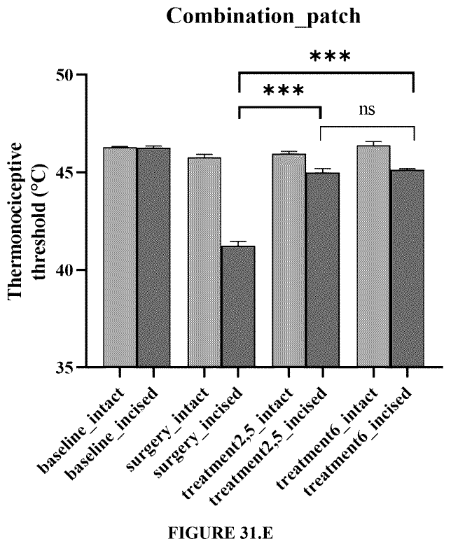

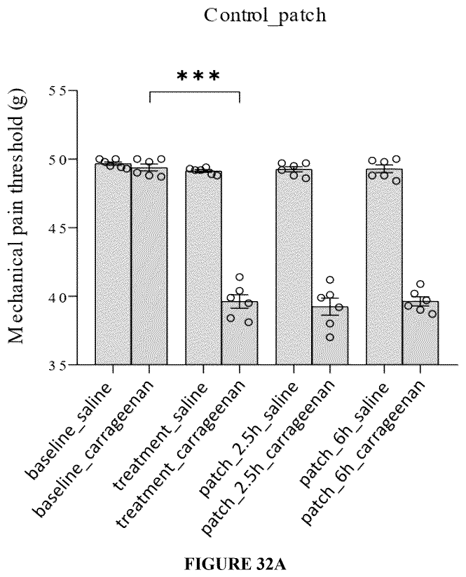

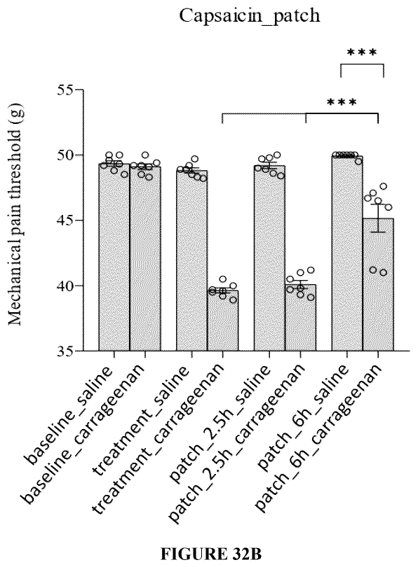

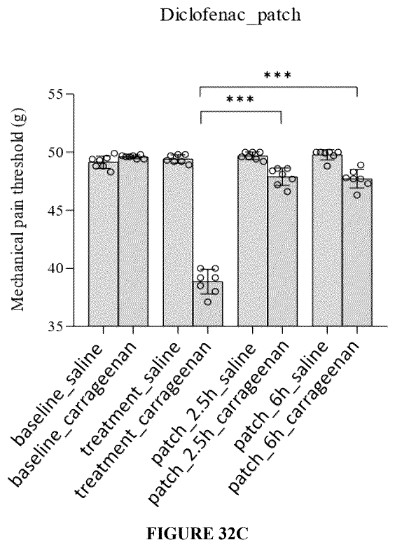

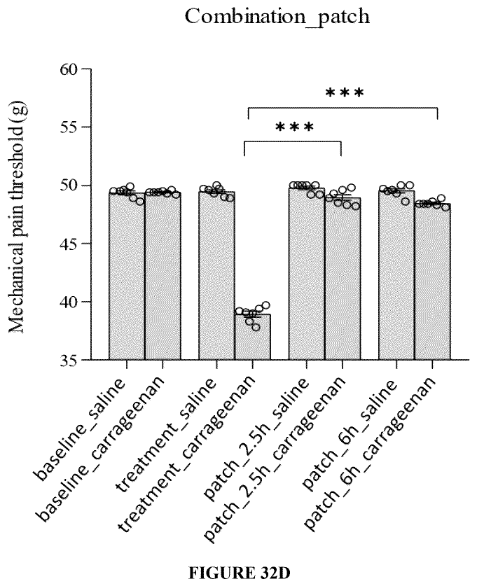

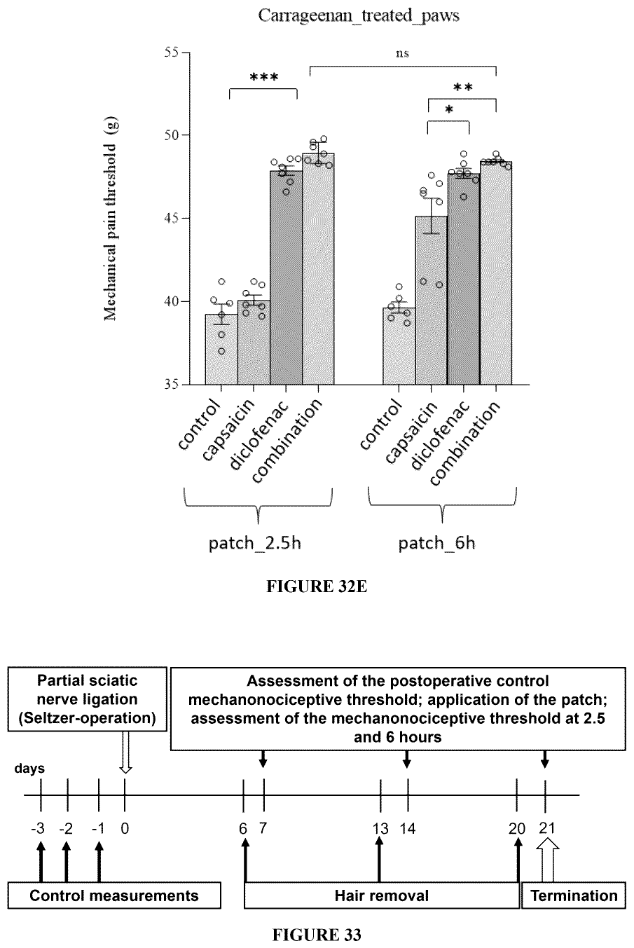

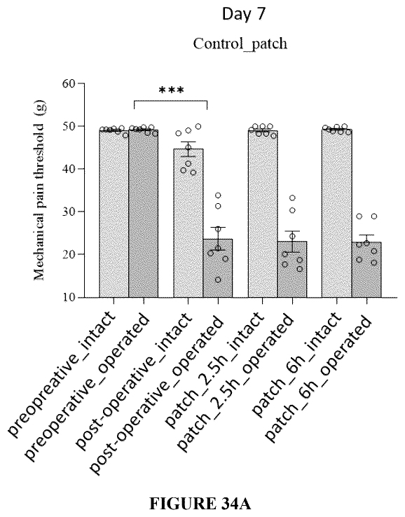

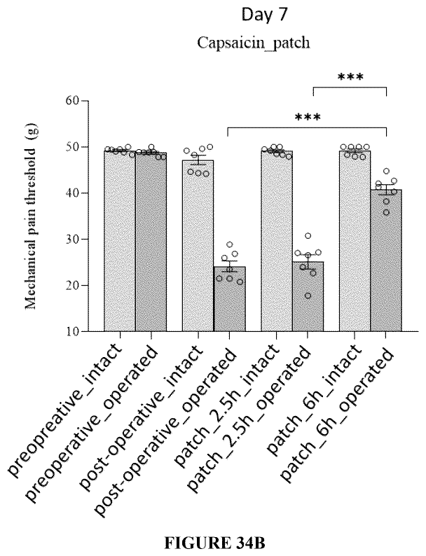

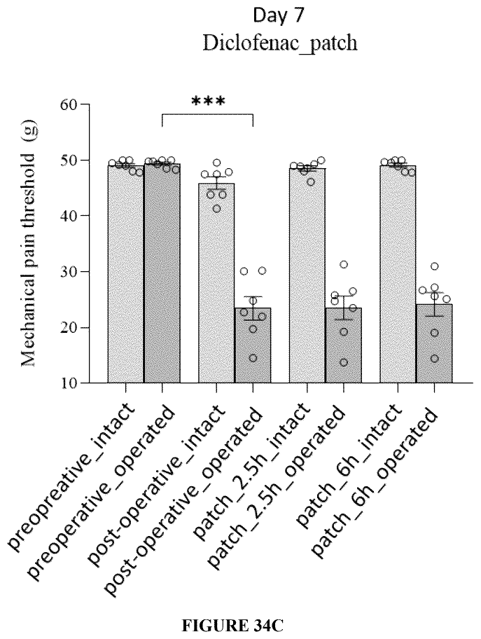

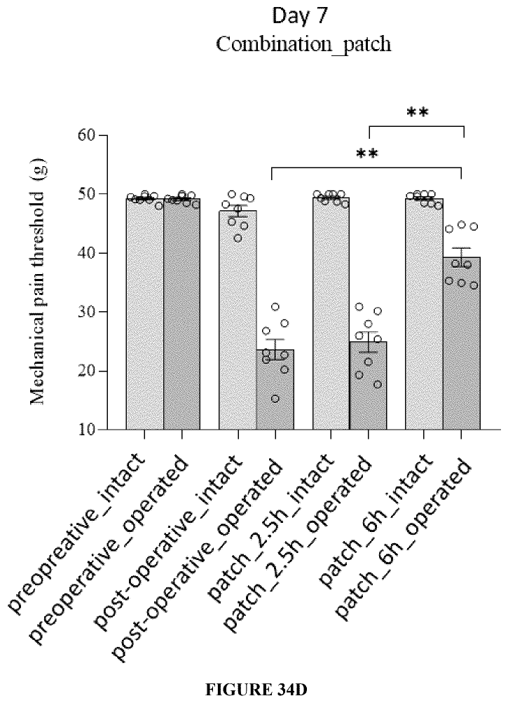

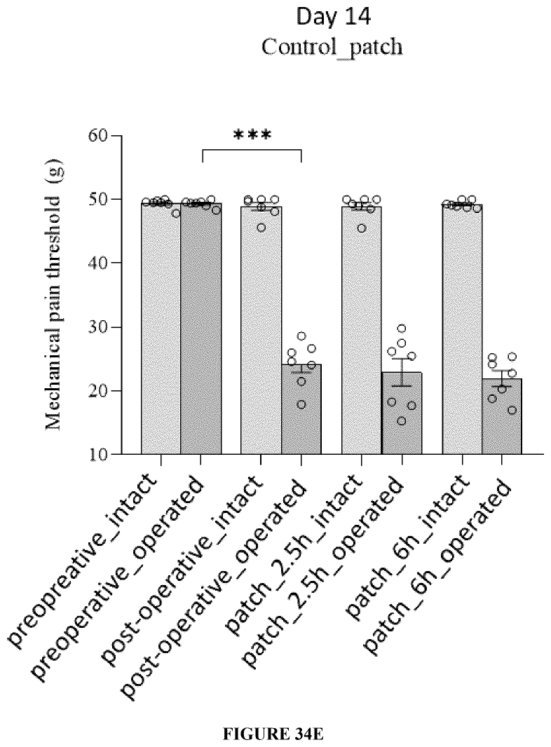

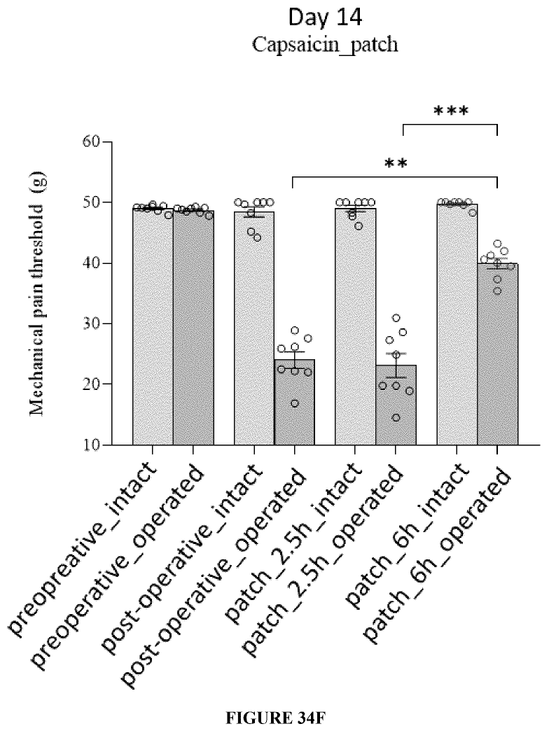

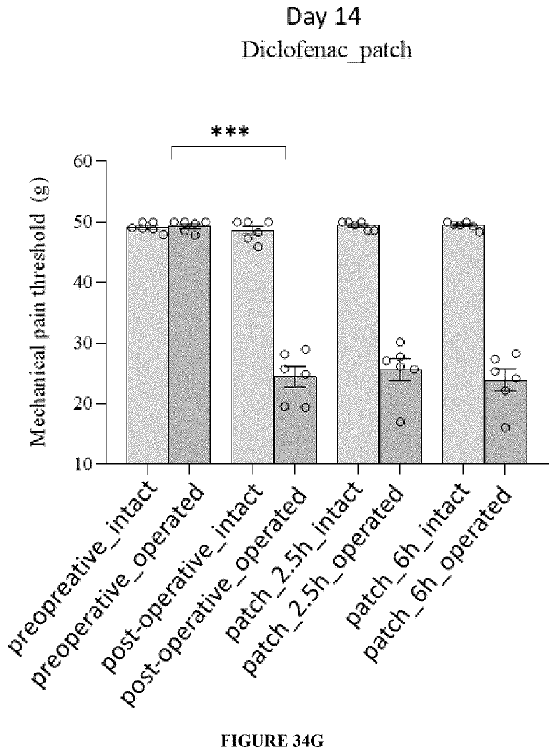

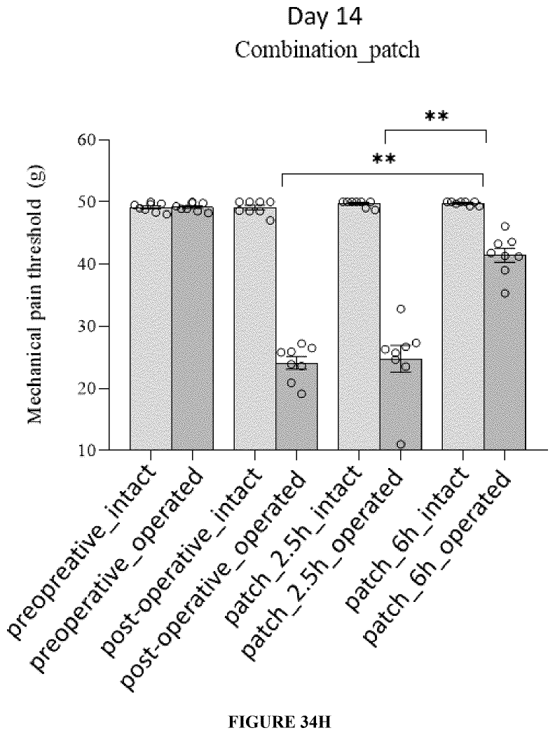

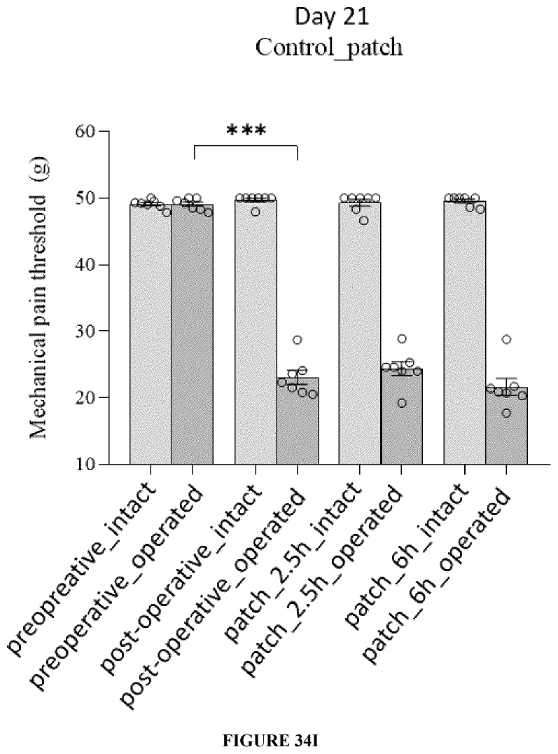

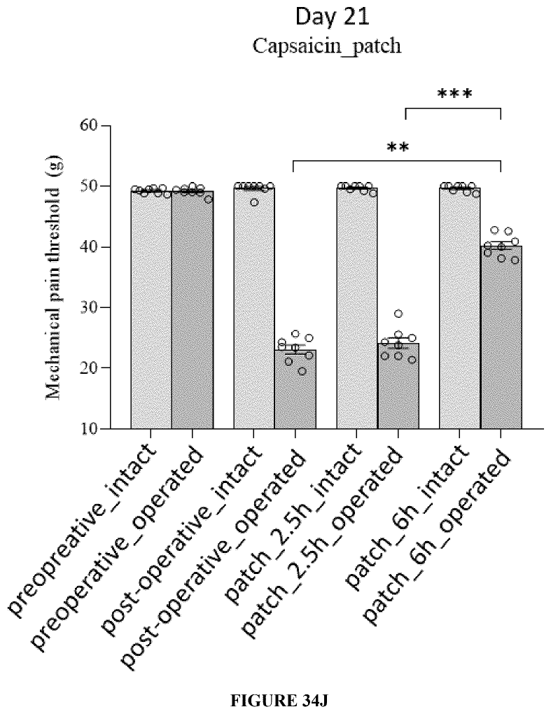

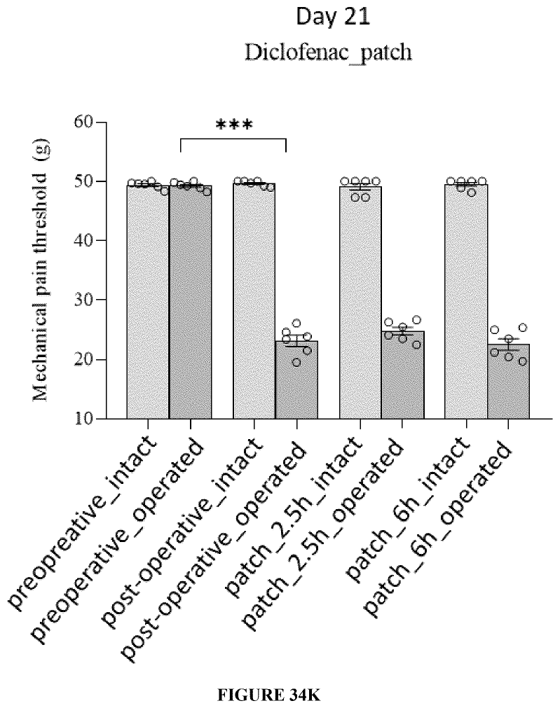

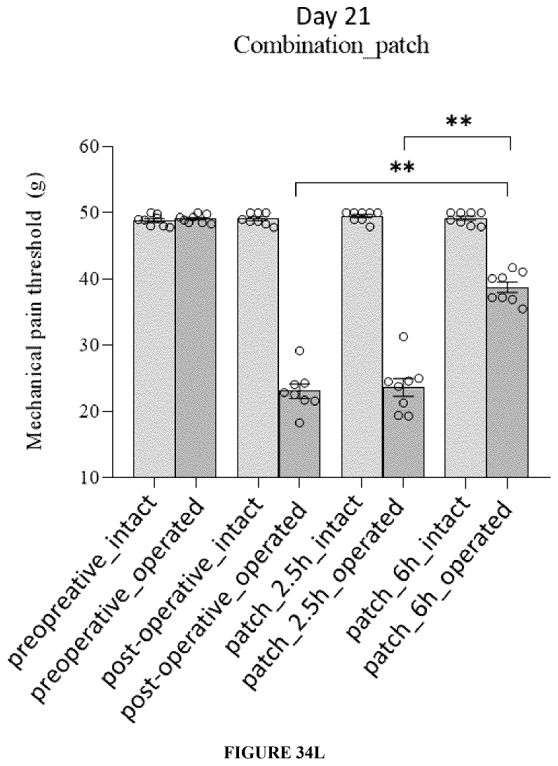

In experiments with delayed application of the patch (the patch was applied 18 hours after the incision), surgical incision of the paws significantly reduced the pain threshold compared to the contralateral intact paws and the respective baseline values, 6 hours after the application of the patch. Control patches without capsaicin did not improve this condition. Capsaicin-releasing patches elevated the thermal pain threshold compared to the control patch. The increased threshold was still lower than the baseline value without incision. Neither the control nor the capsaicin-containing patches changed the heat threshold values of intact paws (FIG. 13).

### Investigation of the Alleviating Effect of Capsaicin-Containing Patches on Carrageenan-Induced Mechanical Allodynia

Carrageenan reduced the mechanical pain threshold, which was detected with a patch applied 18 hours after the challenge, 6 hours after application, in rats treated with control and capsaicin-containing patches, compared to the contralateral paw. Mechanical threshold values of the carrageenan-treated paws were still reduced compared to the contralateral paws after 6 hours of treatment with capsaicin patches or their control. The mechanical pain threshold of the carrageenan-injected paws was significantly elevated by the capsaicin treatment compared to the value detected before the application of the patch. Contralateral paws injected with saline did not show mechanical allodynia (FIG. 14).

### Example 11—Patch Comprising ISDN-Lactose

A powder mixture of isosorbate dinitrate (ISDN) and lactose, as 10.000 g of ISDN-lactose powder dilution of 25%, is measured into a 100 mL Erlenmeyer flask and 40.00 g of glycerol is added to it. The solution is kept at a temperature of 40° C. with constant stirring and is stirred for one hour. After that, the mixture is left to stand for 24 hours, and then it is centrifuged. The pure, active ingredient-saturated glycerol (stock solution I.) is used later.

10% of this material, based on the total weight, is mixed with 80% polydimethylsiloxane-α,ω-diol with a viscosity of 5000 mPas (T-Szilox Hungary, R-5000), then 2% ISDN-lactose powder dilution, 3% M-350 silicone oil and 5% prehydrolyzed tetraethoxysilane (Wacker Chemicals, TES-40) are mixed with it. The homogenized mixture is spread on paper laminated on aluminum foil in a layer thickness of 0.4 mm, then it is crosslinked by placing it in a space containing diethylamine vapors. After a crosslinking time of 20 minutes, the absorbed amine vapor is removed from the layer by placing it in a space with intensive air exchange for 1 hour.

A pressure-sensitive adhesive layer consisting of 3% glycerol, 2% HDK T-30 (Wacker Chemicals) colloidal amorphous silica and 95% MED-1356 PSA solution manufactured by NuSil Technology is layered on the crosslinked layer with a layer thickness of 0.3 mm and the solvent content is allowed to evaporate from the layer.

### Example 12—Patch Comprising ISDN-Lactose

9% of the stock solution I. according to Example 11, based on the total weight, is mixed with 72% (Wacker Chemicals) Elastosil RT-601 A and 8% (Wacker Chemicals) Elastosil RT-601 B components, then 4% ISDN-lactose powder dilution, 2% HDK T-30 (Wacker Chemicals) colloidal amorphous silica and 5% Tween 20 are mixed with it. The homogenized mixture is spread on paper laminated on aluminum foil in a layer thickness of 0.4 mm, and then crosslinked at 70° C. for 15 minutes.

On the layer thus obtained, a homogeneous mixture consisting of 90% R-20000 (T-Szilox, Hungary), 4% propylene glycol, 1% Cab-O-Sil H-300 (Cabot) amorphous colloidal silica and 5% Oxam (T-Szilox, Hungary) cross-linking catalyst is spread, in a layer thickness of 0.2 mm, then crosslinked at room temperature for 40 minutes.

Finally, a pressure-sensitive adhesive layer consisting of 97% MED-1356 PSA solution (NuSil Technology) and 3% glycerol is layered on this layer with a thickness of 0.2 mm and the solvent is allowed to evaporate.

### Example 13—Patch Comprising Diclofenac

1.000 g of diclofenac sodium are weighed into a 100 mL Erlenmeyer flask and 49.00 g of propylene glycol is added. The solution is kept at a temperature of 40° C. with constant stirring and is stirred for one hour. After that, the mixture is left to stand for 24 hours, and then it is centrifuged. The pure, active ingredient-saturated propylene glycol (stock solution II.) is used later.

15% of the stock solution II., based on the total weight, is mixed with 80% polydimethylsiloxane-α,ω-diol with a viscosity of 20,000 mPas, then 1.0% diclofenac sodium, 4% HDK T-30 and 5% Oxam catalyst are mixed with it. The homogenized mixture is spread on a PVC film in a layer thickness of 0.5 mm and crosslinked at room temperature for 30 minutes.

On the layer thus obtained, a homogeneous mixture consisting of 88% R-5000, 7% propylene glycol and 5% Oxam crosslinking catalyst is spread, in a layer thickness of 0.2 mm, then it is crosslinked at room temperature for 40 minutes. Finally, BIO-PSA 7-4301 (Dow Corning) pressure-sensitive silicone adhesive layer is layered on this layer in a thickness of 0.3 mm and the solvent is allowed to evaporate.

### Example 14—Patch Comprising Diclofenac

12% of the stock solution II. according to Example 13, based on the total weight, is mixed with 70% (Wacker Chemicals) Elastosil RT-601 A and 8% (Wacker Chemicals) Elastosil RT-601 B components, then 3% diclofenac sodium, 2% HDK T-30 (Wacker Chemicals) colloidal amorphous silica and 4% Tween 20 and 1% trolamine are mixed with it. The homogenized mixture is spread on a PVC film in a layer thickness of 0.4 mm, then crosslinked at 70° C. for 15 minutes.

Then a pressure-sensitive adhesive layer consisting of 95% MED-1356 PSA solution (NuSil Technology) and 4% PEG-400 and 1% HDK T-30 is layered on this layer in a thickness of 0.4 mm and the solvent is allowed to evaporate.

Data of the patches according to Examples 11 to 14 are summarized in Table 4.

TABLE 4

| | | | | Function of | |
|---|---|---|---|---|---|
| | | | | the given | Possible |
| Example 11 | Example 12 | Example 13 | Example 14 | component | alternatives |
| ISDN-lactose + glycerol | | diclofenac sodium + | diclofenac | active | |
| | | propylene glycol | sodium | ingredient | |
| 80% polydimethyl- | 72% Elastosil | 80% polydimethyl- | 70% Elastosil | raw material of | |
| siloxane-α,ω-diol | RT-601 A | siloxane-α,ω-diol | RT-601 A | silicone layer | |
| with a viscosity of | 8% Elastosil | with a viscosity of | 8% Elastosil | raw material of | |
| 5000 mPas | RT-601 B | 20000 mPas | RT-601 B | silicone layer | |
| 2% ISDN-lactose | 4% ISDN- | 1.0% diclofenac | 3% diclofenac | active | |
| powder dilution | lactose powder | sodium | sodium | ingredient | |
| | dilution | | | | |
| 3% M-350 silicone | | | | liquid apolar | any other methyl |
| oil | | | | excipient | silicone oil with |
| | | | | | viscosity |
| 5% prehydrolyzed | | | | crosslinking | Oxam catalyst |
| tetraethoxysilane + | | | | agent + catalyst | |
| diethylamine | | | | | |
| | 2% HDK T-30 | 4% HDK T-30 | 2% HDK | solid excipient | any other |
| | colloidal | | T-30 colloidal | | hydrophilic |
| | amorphous | | amorphous | | amorphous silica |
| | silica | | silica | | |
| | 5% Tween 20 | | 4% Tween 20 | emulsifier, | |
| | | | | tenside | |
| | | 5% Oxam catalyst | | | 5% prehydrolyzed |
| | | | | | tetra- |
| | | | | | ethoxysilane + |
| | | | | | diethylamine |
| | | | 1% trolamine | emulsifier, | |
| | | | | tenside | |
| | 90% R-20000 | 88% R-5000 | | | |
| | 4% propylene | 7% propylene | | polar liquid | glycerol, PEG-400 |
| | glycol | glycol | | excipient | |
| | 1% Cab-O-Sil | | | solid excipient | |
| | H-300 | | | | |
| | amorphous | | | | |
| | colloidal silica | | | | |
| | 5% Oxam | 5% Oxam | | mixture of | 5% prehydrolyzed |
| | crosslinking | crosslinking | | crosslinking | tetra- |
| | catalyst | catalyst | | agent + catalyst | ethoxysilane + |
| | | | | | diethylamine |
| 3% glycerol | 3% glycerol | | | liquid excipient | propylene glycol, |
| | | | | | PEG-400 |
| 2% HDK T-30 | | | 1% HDK | solid excipient | any other |
| colloidal | | | T-30 | | hydrophilic |
| amorphous silica | | | | | amorphous silica |
| 95% MED-1356 | 97% | | 95% | pressure- | DUPONT Liveo |
| PSA solution | MED-1356 | | MED-1356 | sensitive | BIO-PSA |
| | PSA solution | | PSA solution | adhesive | 7-4102, or |
| | | | | | 7-4101, or |
| | | | | | 7-4301 |
| | | BIO-PSA 7-4301 | | pressure- | Wacker RT-612 |
| | | | | sensitive | A + B |
| | | | | adhesive | |
| | | | 4% PEG-400 | liquid excipient | glycerol, poly- |
| | | | | | ethylene glycol |

### Example 15

We examined the active ingredient release of the preparations produced according to the examples.

Discs with a diameter of 4.0 cm were cut from the preparations produced according to Examples 11 to 14, then they were placed in a flow-through cell in which the adhesive layer of the preparations was in contact with the dissolving liquid. The liquid volume of the cells was 5.0 mL, the temperature of the liquid was 37° C. and it continuously flowed countercurrently through the liquid space of the cell. The volume of the liquid that flowed through was 20 cm³ per hour. As the dissolving liquid, a 95:5 mixture of pH=7.4 phosphate buffer—glycerol was used in the case of examples 1 and 2, and this was changed to a 3:1 mixture of pH=7.4 phosphate buffer—glycerol in the case of example 3. The concentration of the active ingredients was determined by UV-VJS spectroscopic method, using a calibration curve and a “blind” sample. Samples with the same composition as the given example, but without the active ingredient, were used as “blind” samples. In the case of the “blind” samples, we did not observe an increase in absorbance—i.e. active ingredient release—at the wavelength of the measurement.

The results of the dissolution test of the preparations according to Examples 11 to 14 are shown in FIGS. 21 to 24, respectively.

### Example 16—General Description for Preparing Patches According to the Invention

The TTSs according to the invention, equipped with an adhesive regulator layer, were produced by the following steps:
1. the active ingredient(s) are measured into a small beaker,
2. the calculated amount of ethanol is added, and the active ingredient(s) are dissolved,
3. the calculated amount of glycerol, then the polysorbate are added to the solution, and the mixture is homogenized,
4. the calculated amount of amorphous silica is added to the homogeneous solution, then mixed thoroughly until a homogeneous suspension is obtained,
5. the calculated amount of rubber is added to the suspension, and the solution is mixed to homogeneity,
6. the calculated amount of crosslinker is added to the mixture, and it is mixed to homogeneity,
7. the finished mixture is applied to the carrier at an even pace with a patch-spreading machine set to the appropriate layer thickness,
8. the applied layer is crosslinked in 30-45 minutes with air blowing at 70° C.,
9. the components making up the regulator layer are measured into a small plastic jar and mixed to homogeneity,
10. the regulator layer is applied evenly with the set patch-spreading machine,
11. the applied layer is crosslinked in 30-45 minutes with air blowing at 70° C.,
12. after the finished patch reaches room temperature, it is laminated with silicone protective film.

### Example 17—Production and In Vitro Testing of a Patch Comprising Capsaicin and Diclofenac

The patch comprising capsaicin and diclofenac was prepared by the method described in Example 16.

The measurement table for the patch is shown in Table 5.

TABLE 5
Measurement table for a patch comprising
capsaicin and diclofenac sodium

| PDRT-142/I | | | PDRT-142/II | | |
|---|---|---|---|---|---|
| material | measurement | | material | measurement | |
| capsaicin | 0.1007 | g | glycerol | 0.4056 | g |
| powder | | | | | |
| diclofenac | 0.1177 | g | polysorbate 20 | 0.0526 | g |
| Na | | | | | |
| ethanol | 1.00 | mL | RT612A | 4.0374 | g |
| glycerol | 1.3509 | g | RT612B | 4.4339 | g |
| polysorbate | 0.8034 | g | karlstedt oil | 0.0377 | g |
| 20 | | | | | |
| Cabosil | 0.1291 | g | layer to be spread: 0.1 mm | | |
| RT601A | 9.1994 | g | | | |
| RT601B | 1.0388 | g | capsaicin: | 0.4 | mg/cm² |

| | | | |
|---|---|---|---|
| layer to be spread: 0.5 mm | diclofenac: | 0.4 | mg/cm² |

The active ingredient release properties of the prepared patch were measured in a flow-through cell in vitro dissolution test device, the dissolution data measured after 3 weeks (after the matrix equilibrium has been established, based on our previous measurements) can be seen in Table 6, and they are shown as a curve in FIG. 25.

TABLE 6
Dissolution data

| | |
|---|---|
| Capsaicin | Diclofenac |

| time | mg/cm² | time | mg/cm² |
|---|---|---|---|
| 0 | 0 | 0 | 0 |
| 60 | 0.000281 | 60 | 0.001884 |
| 120 | 0.000688 | 120 | 0.001332 |
| 180 | 0.000578 | 180 | 0.001464 |
| 240 | 0.001164 | 240 | 0.001823 |
| 300 | 0.00082 | 300 | 0.001765 |
| 360 | 0.000853 | 360 | 0.001907 |
| Total dissolved | 0.004384 | Total dissolved | 0.010175 |
| capsaicin | | diclofenac | |
| dissolved active | 1.1 | dissolved active | 2.5 |
| ingredient % | | ingredient % | |

Table 6 shows that 1% to 2% of the active ingredient dissolves in the first 6 hours from the measurement concentration of 0.4 mg/cm². Based on these, it can be predicted that the therapeutic time period can be increased, and that increasing the measurement concentration is unnecessary. But in addition, it is important to note that in the case of TTSs, a complete release of the active ingredient can never be achieved, some (in many cases not just a little, see fentanyl-containing patches) active ingredients always remain in the system.

The small fluctuations shown in FIG. 25 are manifestations of the standard deviation arising from the measurement method, the near zero-order kinetics can be clearly observed in the trend.

### Example 18—In Vivo Study of the Patch Comprising Capsaicin and Diclofenac

In this example, the effectiveness of a transdermal patch comprising capsaicin-diclofenac combined active ingredient, which is not yet available on the market, has been tested both in vitro (see Example 17) and in vivo systems. In addition to traditional in vitro methods (Franz cell and Raman spectroscopy), we also used innovative methods (so-called flow-through cell) to model the components of the skin during the examination of the release and absorption of the active ingredients from the patch. During the in vivo tests, on the one hand, we measured the pain response to a thermal stimulus (thermal allodynia) in a rat model of postoperative pain induced by surgical incision of the plantar skin-muscle of a hind paw, using an increasing-temperature water bath, and on the other hand, we examined the pain threshold to a mechanical stimulus (mechanical hyperalgesia) during acute inflammation induced by carrageenan injected into a hind paw, with a dynamic plantar aesthesiometer. This methodological toolkit is excellent for testing the effectiveness of analgesic drug candidate molecules to be used in transdermal systems.

### 18.1 Animals

During the tests, male Wistar rats weighing 200-250 g were examined in 5-5 groups per model according to the following treatments: naive (no patch), blind patch (without active ingredient), capsaicin-containing patch, diclofenac-containing patch, capsaicin-diclofenac combined active ingredient-containing patch (n=13-27/group). The 3D molecular structure of capsaicin is shown in FIG. 26, and that of diclofenac is shown in FIG. 27. FIG. 28 shows a matrix-controlled transdermal patch.

### 18.2 Plantar Skin-Muscle Incision-Induced Postoperative Pain Model

One of the most accepted and widely used models of pain associated with mechanical tissue injury is the plantar skin-muscle incision-induced postoperative pain model, during which the plantar skin-muscle incision of the hind paw causes a significant decrease in the thermonociceptive threshold (Banik et al., 2006; Pogatzki-Zahn et al., 2007).

After a week of conditioning, we determined the control thermonociceptive threshold of the animals participating in the study using an increasing temperature water bath (FIG. 6 and FIG. 29C). During this, holding the animal gently in hand, first the operated and then the intact hind limb were immersed in a tank full of water, then the water in the tank was heated evenly (at a speed of 24° C./min) starting from 30° C. If the animal showed an avoidance reaction (pulled out/strongly shook the affected limb) or the maximum temperature was reached (51° C., which assuredly does not yet cause tissue damage), we stopped heating the water and the temperature corresponding to the thermonociceptive threshold was recorded from the display (Tékus et al., 2010). The control measurements were performed 3 times, from which an average value was calculated.

The animals were anesthetized with sodium pentobarbital (70 mg/kg). The fur on the back of the rats was cut and epilated. After that, the animal's right hind paw was treated with povidone-iodine, and then a 5 mm longitudinal incision was made in the midline of the sole with a scalpel. Based on the wound, the muscle was separated with tweezers. The wound was closed with a single stitch with a 5-0 suture and was again disinfected with povidone-iodine (Pethö et al., 2017; Brennan et al., 1996) (FIG. 29.A).

The thermonociceptive threshold was determined 18 h after the incision of the sole, i.e. postoperative control measurements were performed. After that, one 3×6 mm silicone patch was attached to the scapula area of the animal using Leukoplast™ (FIG. 29.B). The patches comprised capsaicin, or diclofenac, or both active ingredients, and the blind patch was placebo without active ingredient (László et al., 2022). The additional pain threshold measurements were performed two times after the application of the patch (after 2.5 hours and after 6 hours), during which the patch remained on the animal throughout. For the determination of thermal allodynia, the thermonociceptive threshold values measured after the treatments were compared to the average threshold value measured before the plantar skin-muscle incision.

The flow chart of the experimental protocol is shown in FIG. 30.

### 18.3 Carrageenin-Induced Acute Inflammation Model

Carrageenin is a linear sulfated polysaccharide produced from a seaweed (Chondrus crispus), which, when injected intraplantarly, induces a local, mixed-type inflammatory reaction consisting of neurogenic and non-neurogenic components. Although the exact mediator background and mechanism are not known, prostaglandins clearly play a key role in the development of inflammation and well-defined inflammatory hyperalgesia. The model was originally developed for testing COX inhibitors, but it is widely used, in comparison with them, for testing other anti-inflammatory drug candidate compounds, as well (Bölcskei et al., 2005; Winter et al., 1962; O'Rourke et al., 2008; Shakya et al., 2016).

After a week of conditioning, we determined the basic (control) mechanical nociceptive threshold of the animals participating in the study using a dynamic plantar aesthesiometer (DPA) (FIG. 7). During this, the animals were placed in a 15×15 cm Plexiglas cage with a grid at the bottom, in which they could move freely. With a blunt needle, we applied pressure on the animal's hind paw with gradually increasing force, and when the animal showed an avoidance reaction (withdrew its leg), we read the mechanical nociceptive threshold from the digital display (Sándor et al., 2009). The control measurements were performed 3 times, from which an average value was calculated.

The animals were anesthetized with sodium pentobarbital (70 mg/kg). The fur on the back of the rats was cut and epilated. After that, carrageenin (in 3% physiological saline, 100 μL) was injected (intraplantarly) into the right hind paw of the animals to induce inflammatory pain. An equal amount of physiological saline was injected into the contralateral paw (Morris, 2003; Helyes et al., 2006).

The mechanical nociceptive threshold was determined 18 h after the injection of the sole, i.e. control measurements were performed after the carrageenin treatment. After that, one 3×6 cm silicone patch was attached to the scapula area of the animal using Leukoplast™ (FIG. 29.B). The patches comprised capsaicin, or diclofenac, or both active ingredients, and the blind patch was placebo without active ingredient (László et al., 2022). The additional pain threshold measurements were performed two times after the application of the patch (after 2.5 hours and 6 hours), during which the patch remained on the animal throughout. For the determination of mechanical hyperalgesia, the mechanical nociceptive threshold values measured after the treatments were compared to the average threshold value measured before carrageenin administration.

### 18.4 Results

The incision of the hind paw in all groups of animals resulted in a decrease in the thermonociceptive threshold compared to the contralateral, intact hind paws, i.e. thermal allodynia developed (FIG. 31.A-B). 2.5 hours after the application of the patch comprising only capsaicin, we did not observe any change in the decrease of the heat threshold, however, 6 hours after the application of the patch, the painful heat threshold increased slightly but significantly, i.e. the pain-relieving (analgesic) effect of the patch appeared (FIG. 31.C). In contrast, the patch comprising only diclofenac significantly increased the heat threshold at 2.5 hours (i.e. reduced thermal allodynia), but had no effect 6 hours after application of the patch (FIG. 31.D). In the case of the capsaicin-diclofenac combined active ingredient patch, however, interestingly and unexpectedly, the heat threshold increased greatly and significantly already 2.5 hours after the application of the patch, thus the analgesic effect of the patch was already detectable, and this significant pain-relieving effect remained stably observable 6 hours after the application of the patch (FIG. 31.E). The results detailed above were confirmed in two completely independent series of experiments.

In summary, the capsaicin-diclofenac combined active ingredient patch significantly decreased thermal hyperalgesia induced by plantar skin-muscle incision. During the development of this pain-relieving effect, capsaicin on the one hand enhanced and on the other hand prolonged the effect of diclofenac. Capsaicin-diclofenac combined active ingredient patches are promising and effective candidates for the treatment of analgesia.

The results of the carrageenan-induced paw inflammation model is shown in FIG. 32. As can be seen therein, the carrageenan treatment resulted in a decrease in the mechanical nociceptive threshold compared to the contralateral, intact hind paws (FIG. 32A). 2.5 hours after the application of the patch comprising only capsaicin, we did not observe any change in the decrease of the mechanical pain threshold, however, 6 hours after the application of the patch, the mechanical pain threshold increased significantly, i.e. the pain-relieving (analgesic) effect of the patch appeared (FIG. 32B). In contrast, the patch comprising only diclofenac significantly increased the mechanical pain threshold at 2.5 hours, which effect was still observed at 6 hours after the application of the patch (FIG. 32C). In case of the capsaicin-diclofenac combined active ingredient patch, the mechanical pain threshold increased greatly and significantly already 2.5 hours after the application of the patch, thus the analgesic effect of the patch was already detectable, and this significant pain-relieving effect remained stably observable 6 hours after the application of the patch (FIG. 32D). FIG. 32E shows the effect of the different treatments side by side.

### Example 19—In Vivo Study of the Patch Comprising Capsaicin and/or Diclofenac in a Model of Chronic Neuropathic Pain

### Method

### Chronic Neuropathic Pain Model Induced by Partial Sciatic Nerve Ligation (PSNL, Seltzer Operation)

Partial ligation of the sciatic nerve is a reliable and widely used disease model of traumatic neuropathic pain in rodents. The surgical intervention results in significant damage of the thinly myelinated and unmyelinated fibres, leading to abnormal sensory functions such as hyperalgesia, without paralysis of motor functions [Seltzer et al., 1990].

After a week of conditioning (acclimatization to hand-holding as well as to the measuring device), the baseline (control) mechanonociceptive threshold of the animals was determined with dynamic plantar aesthesiometer (DPA, Ugo Basile 37400, Comerio, Italy). Animals were placed in a 15×15 cm Plexiglas cage with a bottom grid, in which they were allowed to move freely. A blunt-end needle was used to apply gradually increasing pressure to the hindpaws of the animals, and when the animal showed a withdrawal response (pulled its hindpaw away), the mechanonociceptive threshold was read from a digital counter. Control measurements were performed on three occasions and a mean value was calculated.

Before the Seltzer operation, the animals were anaesthetised with sodium pentobarbital (70 mg/kg). During PSNL, a tight knot was tied around approximately ⅓-½ of the diameter of the sciatic nerve with 6/0 silk surgical thread [Seltzer et al., 1990; Helyes et al., 2003; Kozsurek et al., 2023]. The skin was secured with interrupted stitches using 4/0 suture thread, and the wound site was disinfected with povidone-iodine solution at the end of surgery.

On the days prior to the postoperative DPA measurements, the hair on the backs of the rats was trimmed and epilated under anaesthesia with sodium pentobarbital (70 mg/kg). On postoperative days 7, 14 and 21, the mechanonociceptive threshold was determined, i.e. postoperative control measurements were performed. Subsequently, a silicone transdermal patch (3×6 mm) was applied to the scapular region of the animal. The patches contained capsaicin (1%), or diclofenac (1 mg/cm²), or both, and the blind patch was a drug-free placebo [László et al., 2022]. Further pain threshold measurements were performed twice after application of the patch (at 2.5 h and 6 h), and the patch remained on the animal throughout the whole period of examination. Mechanonociceptive thresholds measured before and after patch application were expressed as a percentage of the preoperative control values [Bölcskei et al., 2005; Sándor et al., 2009].

FIG. 33 summarizes the experimental procedures of the chronic neuropathic pain model induced by partial sciatic nerve ligation.

### Results

The development of neuropathic pain was assessed over 3 weeks and measurements were taken on days 7, 14 and 21 postoperatively. On each measurement day, first, we determined the postoperative control thresholds. The partial ligation of the sciatic nerve caused a significant decrease in the mechanical pain threshold (mechanonociceptive threshold), i.e. the development of mechanical hyperalgesia, which was already detectable on postoperative day 7 and remained stable throughout the whole study period, i.e. until day 21. The animals that developed neuropathy were identified and only those animals were included in further studies.

During application of the blind patch, the degree of mechanical hyperalgesia measured on the operated limb remained similar to the postoperative control values at both 2.5 hours and 6 hours after application of the patch (FIGS. 34A, 34E and 34I). In the group treated with the patch containing only capsaicin, there was no change at 2.5 hours, but there was a significant decrease in mechanical hyperalgesia at 6 hours after patch application (FIGS. 34B, 34F and 34J). In animals treated with the patch containing only diclofenac, there was no change in mechanical hyperalgesia at 2.5 hours or 6 hours after patch application (FIGS. 34C, 34G and 34K). In the group treated with the combined capsaicin-diclofenac containing patch, similarly to the capsaicin-only patch, a significant decrease in hyperalgesia was measured at 6 h, but not at 2.5 h (FIGS. 34D, 34H and 34L).

### INDUSTRIAL APPLICABILITY OF THE INVENTION

The solution according to the invention is particularly suitable for transdermal uniform, long-lasting (preferably 4 to 24 hours, more preferably 6 to 12 hours) delivery into the skin of active ingredients, preferably small organic active ingredients soluble in polar or apolar, preferably in a polar solvent, preferably in monohydric or polyhydric alcohols, particularly in dihydric or polyhydric alcohols, e.g. in dihydric or trihydric alcohols. preparation.

The invention is particularly preferred for the implementation of a transdermal patch comprising low-dose capsaicin, which, when placed on the skin, ensures the release of the active ingredient over a long period of time, preferably for 4-24 hours, more preferably for 6-12 hours, with near zero-order kinetics.

The invention is also suitable for the production of these preparations. The invention is particularly suitable for the use of these preparations for pain relief or e.g. as a warm-up patch during sports activities.

### REFERENCES

Alexandru D P Papoiu & Gil Yosipovitch Topical capsaicin. The fire of a ‘hot’ medicine is reignited, Expert Opinion on Pharmacotherapy, 2010, 11:8, 1359-1371, DOI: 10.1517/14656566.2010.481670
Alkilani A Z, McCrudden M T, Donnelly R F. Transdermal Drug Delivery: Innovative Pharmaceutical Developments Based on Disruption of the Barrier Properties of the stratum corneum. Pharmaceutics. 2015; 7(4):438-470. Published 2015 Oct. 22. doi:10.3390/pharmaceutics7040438
Anand, P.; Bley, K. Topical Capsaicin for Pain Management: Therapeutic Potential and Mechanisms of Action of the New High-Concentration Capsaicin 8% Patch. British Journal of Anaesthesia 2011, 107, 490-502, doi:10.1093/bja/aer260.
Anderson, Mattias; Afewerki, Samson; Berglund, Per; Córdova Armando Total Synthesis of Capsaicin Analogues from Lignin-Derived Compounds by Combined Heterogeneous Metal, Organocatalytic and Enzymatic Cascades in One Pot. Advanced Synthesis and Catalysis 2014 356(9) 2113-2118 https://doi.org/10.1002/adsc.201301148
Bakonyi, M.; Gácsi, A.; Kovács, A.; Szücs, M.-B.; Berkó, S.; Cśanyi, E. Following-up skin penetration of lidocaine from different vehicles by Raman spectroscopic mapping. J. Pharm. Biomed. Anal. 2018, 154, 1-6, doi:10.1016/j.jpba.2018.02.056.
Banik, R. K., et al. Strain and sex influence on pain sensitivity after plantar incision in the mouse. Anesthesiology, 105(6), 1246-1253. 2006.
Bátai, I. Z.; Horváth, Á.; Pintér, E.; Helyes, Z.; Pozsgai, G. Role of Transient Receptor Potential Ankyrin 1 Ion Channel and Somatostatin Sst4 Receptor in the Antinociceptive and Anti-Inflammatory Effects of Sodium Polysulfide and Dimethyl Trisulfide. Front Endocrinol (Lausanne) 2018, 9, doi:10.3389/fendo.2018.00055.
Berkó, S.; Zsikó, S.; Deák, G.; Gácsi, A.; Kovács, A.; Budai-Szücs, M.; Pajor, L.; Bajory, Z.; Csányi, E. Papaverine hydrochloride containing nanostructured lyotropic liquid crystal formulation as a potential drug delivery system for the treatment of erectile dysfunction. Drug Des. Devel. Ther. 2018, Volume 12, 2923-2931, doi:10.2147/DDDT.S168218.
Bley, K. R. TRPV1 Agonist Approaches for Pain Management. In Vanilloid Receptor TRPV1 in Drug Discovery: Targeting Pain and Other Pathological Disorders; 2010; pp. 325-347.
Bölcskei, K., et al. (2005) Investigation of the role of TRPV1 receptors in acute and chronic nociceptive processes using gene-deficient mice. Pain. 117:368-376.
Brennan T. J., Vandermeulen E. P., Gebhart G. F. Characterization of a Rat Model of Incisional Pain. Pain. 1996; 64:493-502. doi: 10.1016/0304-3959(95)01441-1.
CA02314326 számú kanadai szabadalom
Cameron, M.; Gagnier, J. J.; Little, C. V.; Parsons, T. J.; Blümle, A.; Chrubasik, S. Evidence of Effectiveness of Herbal Medicinal Products in the Treatment of Arthritis—Part 1: Osteoarthritis. Phytotherapy Research 2009, 23, 1497-1515, doi:10.1002/ptr.3007.
Chrubasik, S.; Weiser, T.; Beime, B. Effectiveness and Safety of Topical Capsaicin Cream in the Treatment of Chronic Soft Tissue Pain. Phytotherapy Research 2010, 24, 1877-1885, doi:10.1002/ptr.3335.
De Leon-Casasola, O. New Developments in the Treatment Algorithm for Peripheral Neuropathic Pain. Pain Medicine 2011, 12, S100-S108, doi:10.1111/j.1526-4637.2011.01160.x.
Deal, C. L.; Schnitzer, T. J.; Lipstein, E.; Seibold, J. R.; Stevens, R. M.; Levy, M. D.; Albert, D.; Renold, F. Treatment of Arthritis with Topical Capsaicin: A Double-Blind Trial. Clin Ther 1991, 13, 383-395.
Ercan N, Uludag M O, Agis E R, Demirel-Yilmaz E. The anti-inflammatory effect of diclofenac is considerably augmented by topical capsaicinoids-containing patch in carrageenan-induced paw oedema of rat. Inflammopharmacology. 2013 December; 21(6):413-9. doi:
Füredi, R.; Bölcskei, K.; Szolcsányi, J.; Pethö, G. Effects of Analgesics on the Plantar Incision-Induced Drop of the Noxious Heat Threshold Measured with an Increasing-Temperature Water Bath in the Rat. European Journal of Pharmacology 2009, 605, 63-67, doi:10.1016/j.ejphar.2008.12.035.
GBD 2019 Diseases and Injuries Collaborators (2020). Global burden of 369 diseases and injuries in 204 countries and territories, 1990-2019: a systematic analysis for the Global Burden of Disease Study 2019. Lancet (London, England), 396(10258), 1204-1222. https://doi.org/10.1016/S0140-6736(20)30925-9
Guedes, V.; Castro, J. P.; Brito, I. Topical Capsaicin for Pain in Osteoarthritis: A Literature Review. Reumatol Clin 2018, 14, 40-45, doi:10.1016/j.reuma.2016.07.008.
Hayes A G, Oxford A, Reynolds M, Shingler A H, Skingle M, Smith C, Tyers M B. The effects of a series of capsaicin analogues on nociception and body temperature in the rat. Life Sci. 1984 Mar. 26; 34(13):1241-8. doi: 10.1016/0024-3205(84)90546-0. PMID: 6708727.
Helyes Z, Németh J, Thán M, Bölcskei K, Pintér E, Szolcsányi J. Inhibitory effect of anandamide on resiniferatoxin-induced sensory neuropeptide release in vivo and neuropathic hyperalgesia in the rat. Life Sci. 2003 Sep. 19; 73(18):2345-53.
Helyes Z, Pintér E, Németh J, Sándor K, Elekes K, Szabó A, Pozsgai G, Keszthelyi D, Kereskai L, Engström M, Wurster S, Szolcsányi J. Effects of the somatostatin receptor subtype 4 selective agonist J-2156 on sensory neuropeptide release and inflammatory reactions in rodents. Br J Pharmacol. 2006 October; 149(4):405-15. doi: 10.1038/sj.bjp.0706876.
Helyes, Z.; Pozsgai, G.; Börzsei, R.; Németh, J.; Bagoly, T.; Márk, L.; Pintér, E.; Tóth, G.; Elekes, K.; Szolcsányi, J.; et al. Inhibitory Effect of PACAP-38 on Acute Neurogenic and Non-Neurogenic Inflammatory Processes in the Rat. Peptides 2007, 28, 1847-1855, doi:10.1016/j.peptides.2007.07.001.
Hoffman, A. S. The origins and evolution of “controlled” drug delivery systems. J Control Release. 2008 Dec. 18; 132(3):153-63. doi: 10.1016/j.jconrel.2008.08.012. Epub 2008 Aug. 28.
Horváth, K.; Boros, M.; Bagoly, T.; Sándor, V.; Kilár, F.; Kemény, Á.; Helyes, Z.; Szolcsányi, J.; Pintér, E. Analgesic Topical Capsaicinoid Therapy Increases Somatostatin-like Immunoreactivity in the Human Plasma. Neuropeptides 2014, 48, 371-378, doi:10.1016/j.npep.2014.10.001.
Huang X F, Xue J Y, Jiang A Q, Zhu H L. Capsaicin and its analogues: structure-activity relationship study. Curr Med Chem. 2013; 20(21):2661-72. doi: 10.2174/0929867311320210004. PMID: 23627937.
Kaale, E.; Van Schepdael, A.; Roets, E.; Hoogmartens, J. Determination of Capsaicinoids in Topical Cream by Liquid-Liquid Extraction and Liquid Chromatography. Journal of Pharmaceutical and Biomedical Analysis 2002, 30, 1331-1337, doi:10.1016/S0731-7085(02)00476-4.
Kaiser M. et al. Chapter 23: Capsaicinoids: Occurrence, Chemistry, Biosynthesis, and Biological Effects” in: “Fruit and Vegetable Phytochemicals: Chemistry and Human Health”, Volume I, Second Edition. Edited by Elhadi M. Yahia. © 2018 John Wiley & Sons Ltd. Published 2018 by John Wiley & Sons Ltd.
Kántás, B.; Börzsei, R.; Szöke, É.; Bánhegyi, P.; Horváth, Á.; Hunyady, Á.; Borbély, É.; Hetényi, C.; Pintér, E.; Helyes, Z. Novel Drug-Like Somatostatin Receptor 4 Agonists Are Potential Analgesics for Neuropathic Pain. Int J Mol Sci 2019, 20, doi:10.3390/ijms20246245.
Kecskés, A.; Pohóczky, K.; Kecskés, M.; Varga, Z. V.; Kormos, V.; Szöke, É.; Henn-Mike, N.; Fehér, M.; Kun, J.; Gyenesei, A.; et al. Characterization of Neurons Expressing the Novel Analgesic Drug Target Somatostatin Receptor 4 in Mouse and Human Brains. Int J Mol Sci 2020, 21, doi:10.3390/ijms21207788.
Knotkova H, Pappagallo M, Szallasi A. Capsaicin (TRPV1 Agonist) therapy for pain relief: farewell or revival?Clin J Pain. 2008 February; 24(2):142-54. doi: 10.1097/AJP.0b013e318158ed9e. PMID: 18209521.
Kozsurek, M., Király, K., Gyimesi, K., Lukácsi, E., Fekete, C., Gereben, B., Mohácsik, P., Helyes, Z., Bölcskei, K., Tékus, V., Pap, K., Szücs, E., Benyhe, S., Imre, T., Szabó, P., Gajtkó, A., Holló, K., & Puskár, Z. (2023). Unique, Specific CART Receptor-Independent Regulatory Mechanism of CART (55-102) Peptide in Spinal Nociceptive Transmission and Its Relation to Dipeptidyl-Peptidase 4 (DDP4). International journal of molecular sciences, 24(2), 918. https://doi.org/10.3390/ijms24020918
Kuzma Mónika Dr. Kapszaicinoidok és szalicilátok metabolikus átalakulásainak in vitro és in vivo vizsgálata. Egyetemi doktori értekezés, Pécsi Tudományegyetem Általános Orvostudományi Kar, Gyógyszerészi Kémiai Intézet, Pécs 2016
László S, Bátai IZ, Berkó S, Csányi E, Dombi Á, Pozsgai G, Bölcskei K, Botz L, Wagner Ö, Pintér E. Development of Capsaicin-Containing Analgesic Silicone-Based Transdermal Patches. Pharmaceuticals (Basel). 2022 Oct. 18; 15(10):1279. doi: 10.3390/ph15101279.
Martini, C. H.; Yassen, A.; Krebs-Brown, A.; Passier, P.; Stoker, M.; Olofsen, E.; Dahan, A. A Novel Approach to Identify Responder Subgroups and Predictors of Response to Low- and High-Dose Capsaicin Patches in Postherpetic Neuralgia. European Journal of Pain 2013, 17, 1491-1501, doi:https://doi.org/10.1002/j.1532-2149.2013.00329.x.
Mason, L., Moore, R., Derry, S., Edwards, J. and McQuay, H. (2004) Systematic review of topical capsaicin for the treatment of chronic pain. BMJ 24(328): 991-996
Moon, J.-Y.; Lee, P.-B.; Kim, Y.-C.; Lee, S.-C.; Nahm, F. S.; Choi, E. Efficacy and Safety of 0.625% and 1.25% Capsaicin Patch in Peripheral Neuropathic Pain: Multi-Center, Randomized, and Semi-Double Blind Controlled Study. Pain Physician 2017, 20, 27-35.
Morris C. J. Carrageenan-Induced Paw Edema in the Rat and Mouse. Methods Mol. Biol. 2003; 225:115-121. doi: 10.1385/1-59259-374-7:115.
Ng, S-F.; Rouse, J. J.; Sanderson, F. D.; Meidan, V.; Eccleston; G. M. Validation of a Static Franz Diffusion Cell System for In Vitro Permeation Studies. AAPS PharmSciTech, Vol. 11, No. 3, September 2010 (#2010) DOI: 10.1208/s12249-010-9522-9
O'Rourke A M, et al. (2008) Anti-inflammatory effects of UP 1586 [Z-3-Fluoro-2-(4-methoxybenzyl)allylamine hydocloride], an amine-based inhibitor of semicarbazide-sensitive amine oxidase activity. J Pharmacol Exp Ther 342:867-75.
Pastore, M. N.; Kalia. Y. N.; Horstmann, M.; Roberts, M. S. Transdermal patches: history, development and pharmacology. Br J Pharmacol. 2015 May; 172(9): 2179-2209. Published online 2015 Mar. 18. doi: 10.1111/bph.13059
Peppin John F. and Pappagallo Marco Capsaicinoids in the treatment of neuropathic pain: a review Therapeutic Advances in Neurological Disorder 2014 7(1) 22-32 DOI: 10.1177/1756285613501576
Persson M S M, Stocks J, Walsh D A, Doherty M, Zhang W. The relative efficacy of topical non-steroidal anti-inflammatory drugs and capsaicin in osteoarthritis: a network meta-analysis of randomised controlled trials. Osteoarthritis Cartilage. 2018 December; 26(12):1575-1582. doi:
Pethö, G.; Bölcskei, K.; Füredi, R.; Botz, B.; Bagoly, T.; Pintér, E.; Szolcsányi, J. Evidence for a Novel, Neurohumoral Antinociceptive Mechanism Mediated by Peripheral Capsaicin-Sensitive Nociceptors in Conscious Rats. Neuropeptides 2017, 62, 1-10, doi:10.1016/j.npep.2017.02.079.
Pogatzki-Zahn E M, Zahn P K, Brennan T J. Postoperative pain—clinical implications of basic research. Best Pract Res Clin Anaesthesiol. 21: 3-13. (2007)
Rosenthal, B. M.; Ho, R. H. An Electron Microscopic Study of Somatostatin Immunoreactive Structures in Lamina II of the Rat Spinal Cord. Brain Research Bulletin 1989, 22, 439-451, doi:10.1016/0361-9230(89)90071-3.
Sándor, K.; Elekes, K.; Szabó, Á.; Pintér, E.; Engström, M.; Wurster, S.; Szolcsányi, J.; Helyes, Z. Analgesic Effects of the Somatostatin Sst4 Receptor Selective Agonist J-2156 in Acute and Chronic Pain Models. European Journal of Pharmacology 2006, 539, 71-75, doi:10.1016/j.ejphar.2006.03.082.
Sándor K, Bölcskei K, McDougall J J, Schuelert N, Reglodi D, Elekes K, Petho G, Pintér E, Szolcsányi J, Helyes Z. Divergent peripheral effects of pituitary adenylate cyclase-activating polypeptide-38 on nociception in rats and mice. Pain. 2009 January; 141(1-2):143-50. doi: 10.1016/j.pain.2008.10.028.
Sándor K, Kormos V, Botz B, Imreh A, Bölcskei K, Gaszner B, Markovics A, Szolcsányi J, Shintani N, Hashimoto H, Baba A, Reglodi D, Helyes Z. Impaired nocifensive behaviours and mechanical hyperalgesia, but enhanced thermal allodynia in pituitary adenylate cyclase-activating polypeptide deficient mice. Neuropeptides. 44: 363-71. (2010)
Seltzer, Z.; Dubner, R.; Shir, Y. A novel behavioral model of neuropathic pain disorders produced in rats by partial sciatic nerve injury. Pain 1990, 43, 205-218.
Shakya A K, et al. (2016) Molecular modelling, synthesis, characterization and pharmacological evaluation of benzo[d]oxazole derivates as a non-steroidal anti-inflammatory agents. Saudi Pharm J 24:616-24.
Spampinato, S.; Romualdi, P.; Candeletti, S.; Cavicchini, E.; Ferri, S. Distinguishable Effects of Intrathecal Dynorphins, Somatostatin, Neurotensin and s-Calcitonin on Nociception and Motor Function in the Rat. Pain 1988, 35, 95-104, doi:10.1016/0304-3959(88)90281-3.
Srinivas Nalamachu; Jeffrey Gudin; Characteristics of Analgesic Patch Formulations, Journal of Pain Research 2020:13 2343-2354
Stella Zsikó, Erzsébet Csányi, Anita Kovács, Maria Budai-Szücs, Attila Gácsi, Szilvia Berkó. Novel In Vitro Investigational Methods for Modeling Skin Permeation: Skin PAMPA, Raman Mapping. Pharmaceutics. 2020 Aug. 25; 12(9):803. doi: 10.3390/pharmaceutics12090803.
Szolcsányi, J. Forty Years in Capsaicin Research for Sensory Pharmacology and Physiology. Neuropeptides 2004, 38, 377-384, doi:10.1016/j.npep.2004.07.005.
Tanner, T.; Marks R. Delivering drugs by the transdermal route: review and comment. Skin Research and Technology 2008; 14: 249-260 doi: 10.1111/j.1600-0846.2008.00316.x
Tékus, V., Bölcskei, K., Kis-Varga, A., Dézsi, L., Szentirmay, E., Visegrády, A., Horváth, C., Szolcsányi, J., Petho, G. (2010). Effect of transient receptor potential vanilloid 1 (TRPV1) receptor antagonist compounds SB705498, BCTC and AMG9810 in rat models of thermal hyperalgesia measured with an increasing-temperature water bath. European journal of pharmacology, 641(2-3), 135-141.
Tomanek A. Silicone und Technik, Wacker-Chemie Gmbh, München, 1990
Wagner Ö. A NEW TRANSDERMAL SYSTEM I.; 1991, PERIODICA POLYTECHNICA SER. CHEM. ENG. VOL. 35, NO. 3, PP. 169-176
Wagner O. Development of a New Silicon-Based Transdermal System. I. Study of Silicone Elastomers and Effect of Liquid Ingredients, 1998, Drug Development and Industrial Pharmacy, 24:3, 243-252, DOI: 10.3109/03639049809085616
Winter C A, et al. (1962) Carrageenan-induced edema in hind paw of the rat as an assay for antiinflammatory drugs. Proc Soc Exp Bio Med. 111:544-47.
Zsikó S; Csányi E; Kovács A; Budai-Szücs M; Gácsi A; Berkó S Methods to Evaluate Skin Penetration In Vitro. Sci. Pharm. 2019, 87, 19, doi:10.3390/scipharm87030019.
CN107296852 Chinese patent
HU 197519A Hungarian patent application
HU 210931A Hungarian patent application
HU 217520B Hungarian patent
U.S. Pat. No. 8,821,920B2
U.S. Pat. No. 5,665,378A
WO2004/089361 A1 international patent application
WO2020/263643A1 international patent application

1. Topical, preferably transdermal preparation, for administering an active ingredient topically, said preparation comprising
a silicone matrix comprising a solid active ingredient,
an active ingredient dispersed in the silicone matrix in the form of solid particles,
the saturated solution of the active ingredient in the silicone matrix,
a liquid excipient, which is a solvent in which the active ingredient is dissolved, said liquid excipient controlling the solubility of the active ingredient,
a solid excipient dispersed in the form of granules to ensure homogeneous dispersion of the active ingredient,
wherein the active ingredient leaving the matrix during topical administration, is replaced by the dissolution of the active ingredient dispersed in the form of solid particles in such a way that the solution of the active ingredient remains saturated for at least 4 hours.

2. The transdermal preparation according to claim 1, which comprises the active ingredient in a concentration of at most 1%, preferably at most 0.5%, particularly preferably less than 0.4%.

3. The transdermal preparation according to claim 2, which comprises a capsaicinoid, preferably capsaicin, as an active ingredient in a concentration of 0.05% to 1%, preferably less than 0.4%, particularly preferably in a concentration of 0.1% to 0.35%.

4. The transdermal preparation according to claim 2, for pain relief during topical, preferably percutaneous, administration for at least 4 hours, preferably with prolonged release of the active ingredient for 4 to 24 hours, for 4 to 12 hours, in particular for 6 to 12 hours.

5. The transdermal preparation according to claim 1, in which the excipient controlling the solubility is a polar solvent.

6. The transdermal preparation according to claim 5, in which the excipient controlling the solubility of the active ingredient is selected from the group consisting of monohydric and polyhydric non-volatile alcohols, preferably glycerol, preferably in an amount of 1% to 15%; wherein preferably the excipient is also a solvent,
and wherein the active ingredient is present in the liquid excipient in a dissolved, saturated state, together with the active ingredient present in solid form.

7. The transdermal preparation according to claim 1, which comprises a surfactant as an additional excipient, preferably a non-ionic surfactant, preferably in an amount of 0% to 5%.

8. The transdermal preparation according to claim 1, in which the solid excipient is selected from the group consisting of colloidal silica; and an inactive solid excipient, preferably calcium carbonate and saccharide.

9. The transdermal preparation according to claim 1, which does not comprise a penetration enhancer.

10. A transdermal patch comprising the preparation according to claim 1, which comprises the preparation formed in the form of one or two matrix layer(s), and
comprises a pressure-sensitive adhesive silicone tape layer in contact with the plane of the preparation formed in the form of a layer,
wherein the one or two matrix layer(s) in the preparation are formed in a flat shape, in a layer thickness of at most 0.1 mm to 1 mm, preferably 0.1 mm to 0.8 mm or 0.2 mm to 1 mm, in particular 0.2 mm to 0.6 mm.

11. The transdermal patch according to claim 10, which can release the active ingredient stably for at least 4 hours, preferably for at least 6 hours, with near zero-order kinetics, wherein the patch is formed in the form of multiple layers in contact with each other along their interface, preferably their boundary plane, wherein solid active ingredient, preferably active ingredient, is only introduced into one layer during production,
wherein the active ingredient is preferably selected from the group consisting of
capsaicinoid, preferably capsaicin, non-steroidal anti-inflammatory drug (NSAID), particularly preferably diclofenac, nitrate esters, preferably esters of sugar derivatives, preferably nitrate esters of sugars and sugar derivatives, particularly preferably isosorbide dinitrate (ISDN).

12. The transdermal patch according to claim 11, which comprises a capsaicinoid, preferably capsaicin, and a non-steroidal anti-inflammatory drug (NSAID), preferably diclofenac as active ingredient.

13. The transdermal patch according to claim 12, wherein
the capsaicin is present in a concentration of at most 0.5%, and/or
the diclofenac is present in a concentration of at most 0.5%.

14. The transdermal patch according to claim 10, in which the excipient controlling the solubility of the active ingredient is selected from the group consisting of monohydric and polyhydric non-volatile alcohols, preferably glycerol, preferably in an amount of 1% to 15%.

15. The transdermal patch according to claim 10, which comprises a surfactant as an additional excipient, preferably a non-ionic surfactant, preferably in an amount of 0% to 5%, wherein preferably the liquid and/or solid excipients are present in the silicone matrix in an amount of 4% to 20%.

16. The transdermal patch according to claim 10, in which the silicone matrix, preferably silicone layer, comprising the active ingredient present in solid form comprises a solid excipient selected from the group of amorphous colloidal silica, preferably hydrophilic colloidal silica; and/or inactive solid excipient, preferably calcium carbonate, saccharide, preferably lactose or glucose, wherein preferably the active ingredient is present in the form of pure solid particles, and is in solid granular (powder) form, wherein the size of the particles is 5 microns to 200 microns.

17. The transdermal patch according to claim 10, said patch comprising
a) a silicone matrix layer comprising a solid active ingredient, which comprises the following:
a cross-linking silicone as raw material,
optionally a crosslinker,
an active ingredient, wherein the active ingredient is present both in dissolved and solid form,
a liquid excipient (polar solvent) for dissolving the active ingredient, preferably trihydric alcohol, e.g. glycerol,
optionally an emulsifier/surfactant for the uniform distribution of the polar solvent within the matrix,
optionally a solid excipient; and
b) a pressure-sensitive adhesive layer; and
c) optionally a silicone matrix layer comprising no active ingredient between the silicone matrix layer comprising the active ingredient according to a) and the pressure-sensitive adhesive layer according to b), wherein the silicone matrix layer comprising no active ingredient comprises the following:
a cross-linking silicone as raw material,
a liquid excipient, preferably trihydric alcohol, e.g. glycerol,
optionally a crosslinker,
optionally an emulsifier.

18. The transdermal patch according to claim 10,
wherein the cross-linking silicone raw material is a silicone raw material cross-linking by condensation and/or addition method, preferably polydimethylsiloxane-α,ω-diol.

19. The transdermal patch according to claim 10, in which the active ingredient is a capsaicinoid, wherein preferably the layer comprising the solid active ingredient comprises at most 1% by weight of capsaicinoid or capsaicin as the active ingredient.

20. A method for using a capsaicinoid in prolonging the analgesic effect of a topically applied NSAID, wherein the capsaicin is present in a topical, preferably transdermal preparation in a concentration of at most 1%, preferably at most 0.5%, and
wherein the NSAID is present in a topical, preferably transdermal preparation in a concentration of at most 1%, preferably at most 0.5%,
said method comprising the steps of
applying the transdermal preparation comprising the capsaicin on the skin of a patient in need of prolonging the analgesic effect of a topically applied NSAID, for at least 4 hours, and
applying the transdermal preparation comprising the NSAID on the skin of the patient, for at least 4 hours.

21. The method for using the capsaicinoid in prolonging the analgesic effect of topically applied diclofenac according to claim 20, wherein the capsaicinoid and the NSAID are each present in a preparation
comprising
a silicone matrix comprising a solid active ingredient,
an active ingredient dispersed in the silicone matrix in the form of solid particles,
the saturated solution of the active ingredient in the silicone matrix,
a liquid excipient, which is a solvent in which the active ingredient is dissolved, said liquid excipient controlling the solubility of the active ingredient,
a solid excipient dispersed in the form of granules to ensure homogeneous dispersion of the active ingredient,
wherein the active ingredient leaving the matrix during topical administration, is replaced by the dissolution of the active ingredient dispersed in the form of solid particles in such a way that the solution of the active ingredient remains saturated for at least 4 hours;
as a solid active ingredient in the silicone matrix comprising the solid active ingredient and in a saturated solution, said method comprising the step of applying the transdermal preparation(s) on the skin for at least 4 hours.

22. The method for using the capsaicinoid in prolonging the analgesic effect of topically applied diclofenac according to claim 21, wherein the capsaicinoid and the NSAID are each present in a transdermal patch comprising
the preparation comprising
a silicone matrix comprising a solid active ingredient,
an active ingredient dispersed in the silicone matrix in the form of solid particles,
the saturated solution of the active ingredient in the silicone matrix,
a liquid excipient, which is a solvent in which the active ingredient is dissolved, said liquid excipient controlling the solubility of the active ingredient,
a solid excipient dispersed in the form of granules to ensure homogeneous dispersion of the active ingredient,
wherein the active ingredient leaving the matrix during topical administration, is replaced by the dissolution of the active ingredient dispersed in the form of solid particles in such a way that the solution of the active ingredient remains saturated for at least 4 hours;
which comprises the preparation formed in the form of one or two matrix layer(s), and
comprises a pressure-sensitive adhesive silicone tape layer in contact with the plane of the preparation formed in the form of a layer,
wherein the one or two matrix layer(s) in the preparation are formed in a flat shape, in a layer thickness of at most 0.1 mm to 1 mm, preferably 0.1 mm to 0.8 mm or 0.2 mm to 1 mm, in particular 0.2 mm to 0.6 mm;
as a solid active ingredient in the silicone matrix comprising the solid active ingredient and in a saturated solution, said method comprising the step of applying the transdermal patch(es) on the skin of the patient for at least 4 hours.

23. The method for using the capsaicinoid in prolonging the analgesic effect of topically applied diclofenac according to claim 20, wherein the capsaicinoid is capsaicin and the NSAID is diclofenac and capsaicin and diclofenac are administered simultaneously, and the capsaicin and diclofenac are both present in the same preparation comprising
a silicone matrix comprising a solid active ingredient,
an active ingredient dispersed in the silicone matrix in the form of solid particles,
the saturated solution of the active ingredient in the silicone matrix,
a liquid excipient, which is a solvent in which the active ingredient is dissolved, said liquid excipient controlling the solubility of the active ingredient,
a solid excipient dispersed in the form of granules to ensure homogeneous dispersion of the active ingredient,
wherein the active ingredient leaving the matrix during topical administration, is replaced by the dissolution of the active ingredient dispersed in the form of solid particles in such a way that the solution of the active ingredient remains saturated for at least 4 hours
said method comprising the step of applying the transdermal preparation on the skin for at least 4 hours.

24. The method for using the capsaicinoid in prolonging the analgesic effect of topically applied diclofenac according to claim 20, wherein the capsaicinoid is capsaicin and the NSAID is diclofenac, and capsaicin and diclofenac are administered simultaneously, and the capsaicin and diclofenac are present in the same transdermal patch comprising
the preparation comprising
a silicone matrix comprising a solid active ingredient,
an active ingredient dispersed in the silicone matrix in the form of solid particles,
the saturated solution of the active ingredient in the silicone matrix,
a liquid excipient, which is a solvent in which the active ingredient is dissolved, said liquid excipient controlling the solubility of the active ingredient,
a solid excipient dispersed in the form of granules to ensure homogeneous dispersion of the active ingredient,
wherein the active ingredient leaving the matrix during topical administration, is replaced by the dissolution of the active ingredient dispersed in the form of solid particles in such a way that the solution of the active ingredient remains saturated for at least 4 hours;
which comprises the preparation formed in the form of one or two matrix layer(s), and
comprises a pressure-sensitive adhesive silicone tape layer in contact with the plane of the preparation formed in the form of a layer,
wherein the one or two matrix layer(s) in the preparation are formed in a flat shape, in a layer thickness of at most 0.1 mm to 1 mm, preferably 0.1 mm to 0.8 mm or 0.2 mm to 1 mm, in particular 0.2 mm to 0.6 mm;
said method comprising the step of applying the transdermal patch on the skin for at least 4 hours.

25. The method for using the capsaicin in prolonging the analgesic effect of topically applied diclofenac according to claim 24, said method comprising the step of applying the transdermal preparation on the skin for at least 6 hours with near zero-order kinetics.

26. The method for using the capsaicinoid in prolonging the analgesic effect of topically applied diclofenac according to claim 20, wherein the capsaicinoid is present in a preparation comprising
a silicone matrix comprising a solid active ingredient,
an active ingredient dispersed in the silicone matrix in the form of solid particles,
the saturated solution of the active ingredient in the silicone matrix,
a liquid excipient, which is a solvent in which the active ingredient is dissolved, said liquid excipient controlling the solubility of the active ingredient,
a solid excipient dispersed in the form of granules to ensure homogeneous dispersion of the active ingredient,
wherein the active ingredient leaving the matrix during topical administration, is replaced by the dissolution of the active ingredient dispersed in the form of solid particles in such a way that the solution of the active ingredient remains saturated for at least 4 hours;
as a solid active ingredient in the silicone matrix comprising the solid active ingredient and in a saturated solution, said method comprising the step of applying the transdermal preparation on the skin for at least 6 hours.

27. The method for using the capsaicinoid in prolonging the analgesic effect of topically applied diclofenac according to claim 20, wherein analgesic effect is pain relief wherein pain is selected from the group consisting of
acute pain,
chronic inflammatory pain,
neuropathic pain.

28. The method according to claim 27, wherein the pain is peripheral neuropathic pain.

29. The method according to claim 27, wherein pain relief limited to the time of application of the transdermal preparation, and wherein the preparation is applied for 6 to 12 hours, with prolonged, preferably near zero-order kinetic release of the active ingredient.
